(19) United States
(12) Patent Application Publication
(10) Pub. No.: US 2026/0284204 A1
(43) Pub. Date: Sep. 24, 2026

(54) UNIVERSAL ABT COMPOUNDS AND USES THEREOF

(71) Applicant: KLEO PHARMACEUTICALS, INC., New Haven, CT (US)

(72) Inventors: David Adam Spiegel, New Haven, CT (US); Matthew Ernest Welsch, New Haven, CT (US)

(21) Appl. No.: 19/677,891

(22) Filed: May 14, 2026

Related U.S. Application Data
(63) Continuation of application No. 16/634,032, filed on Jul. 26, 2018, now abandoned.
(60) Provisional application No. 62/537,034, filed on Jul. 26, 2017.

(51) Int. Cl. A61K 47/55; A61K 38/00; A61K 47/54; A61P 35/00; C07K 5/062; C07K 5/072; C07K 5/078; C07K 5/087; C07K 5/103; C07K 5/11; C07K 7/06; C07K 7/08; C07K 7/56; C07K 16/28

(52) U.S. Cl. CPC A61K 47/551; A61K 47/542; A61K 47/557; A61P 35/00; C07K 5/0606; C07K 5/06121; C07K 5/06139; C07K 5/0812; C07K 5/1008; C07K 5/1019; C07K 7/06; C07K 7/08; C07K 7/56; C07K 16/283; A61K 38/00; C07K 2317/732; C07K 2319/33

(57) ABSTRACT

Among other things, the present disclosure provides compounds comprising universal antibody binding moieties and targeting moieties. In some embodiments, provided compounds recruit various types of antibodies to diseased cells such as cancer cells, and induce immune activities to kill such cells. Provided technologies are useful for treating various diseases including cancer.

### CROSS-REFERENCE TO RELATED APPLICATIONS

This application claims priority to U.S. Provisional Application No. 62/537,034, filed Jul. 26, 2017, the entirety of which is incorporated herein by reference.

### TECHNICAL FIELD

The present invention relates to compounds and methods useful for recruiting antibodies to cancer cells. The invention also provides pharmaceutically acceptable compositions comprising compounds of the present invention and methods of using said compositions in the treatment of various disorders.

### BACKGROUND

Immune system activities may be utilized to prevent or treat various conditions, disorders and diseases.

### SUMMARY

In some embodiments, the present disclosure provides technologies, e.g., compounds, compositions, methods, etc., that are particularly useful for recruiting antibodies to damaged or defective tissues (e.g., tumors, certain wounds, etc.), foreign objects or entities (e.g., infectious agents), etc. In some embodiments, provided technologies can trigger, generate, encourage, and/or enhance immune system activities toward target cells, tissues, objects and/or entities, for example, antibody-dependent cell-mediated cytotoxicity (ADCC), antibody-dependent cellular phagocytosis (ADCP), etc. In some embodiments, the present disclosure is directed to the design and synthesis of a new small-molecule capable of redirecting endogenous antibodies selectively to diseased cells, e.g., cancer cells, and inducing immune system activities, e.g., an antibody-directed, cell-mediated immune response, e.g., cytotoxicity, ADCP, etc.

Although still in its infancy, the concept of using small molecules to template the human immune response has shown realistic potential. Recent reports have surfaced in which small molecules have been used to direct antibodies to cancerous cells such as breast carcinoma cells, melanoma cells, and nasopharyngeal epidermal carcinoma cells. Animal studies have demonstrated that these molecules can promote tumor rejection and antitumor immunity in mice. In some embodiments, such molecules can promote tumor regression and/or inhibit tumor growth. Because this process allows for the direction of endogenous antibodies selectively to the cell of interest, it has the potential to harness the power of many immunotherapies, e.g., monoclonal antibody (mAb)-based therapeutics, while limiting the costs and side effects associated with administering exogenous antibodies. By developing similar methods which recruit antibodies to diseased cells, e.g., cancer cells, the proposed research will help broaden this field while potentially creating new therapies for various diseases.

In some embodiments, the present disclosure provides antibody recruiting molecules, which comprise antibody binding moieties and target binding moieties optionally through linker moieties. In some embodiments, antibody recruiting molecules (ARMs) are a class of compounds composed of two functional segments connected by a linker—a target binding terminus (TBT) and an antibody binding terminus (ABT). A target binding moiety, e.g., a target binding terminus can confer specificity of an ARM to its target, e.g., a diseased cell of interest, through, e.g., binding a receptor differentiating a target from a non-target (e.g., diseased cells from other cell types). Among other things, ARMs can enable target-specific recruitment of antibodies, e.g., endogenous antibodies, administered antibodies, etc., through ABTs, and/or trigger, generate, encourage, and/or enhance immune activities, e.g., immune-mediated killing of target cells. Without the intention to be bound by any particular theory, it is reported that previous work in the Spiegel lab has shown that ARM directed killing is predominately mediated through natural killer (NK) cells and macrophages, whose principal receptor involved in this process is CD16a (or FCgammaRIIIa).

Previously reported ABTs, e.g., ABTs explored in the Spiegel lab, have been focused on molecules that bind to the variable region of an antibody Fab (antigens). Among other things, the present disclosure encompasses the recognition that success of this approach therapeutically is contingent upon there being a sufficient level of a specific antibody population present, which may vary dramatically among individuals. In some embodiments, the present disclosure provides technologies that can circumvent the dependence of specific antibody populations and undesirable effects that may result from individual variations of the specific antibody populations. Particularly, in some embodiments, the present disclosure provides ARMs comprising ABTs that can bind to Fc region of antibodies and thereby can, among other things, recruit antibodies of various antigen-specificity (“universal ABTs”, or “uABTs”). In some embodiments, Applicant describes the utilization of a class of ABTs that bind to a conserved site present in the F_{c} region of IgG. In some embodiments, uABTs enables recruitment of all IgG subclasses (IgG1, IgG2, IgG3, IgG4). In some embodiments, uABTs enables recruitment preferentially of IgG1, IgG2, and/or IgG4. In some embodiments, recruitment of antibodies, e.g., IgG subclasses, is only limited by the administered dose of an ARM, and/or is not by levels of antibodies having a particular Fab region in an individual.

To provide ARMs comprising uABTs, Applicant evaluated a multitude of peptides that have been reported to bind to human IgG Fc for their applicability to be utilized in the ARM platform. In some embodiments, an essential component of assessing the therapeutic utility of this strategy is demonstrating that antibodies recruited in this orientation are capable of binding to, and activating CD16a.

Biochemical and cell-based assays demonstrate that a series of Fc-binding cyclic peptides are indeed capable of binding antibodies in a manner that is conducive to CD16a activation and are applicable to the ARM platform. In some embodiments, the present disclosure demonstrates that uABTs can bind to a variety of antibodies. In some embodiments, in addition to affinity for all human IgG subclasses, in exploring different methods of evaluating these peptides are highly species cross reactive—binding to secondary antibodies from goat, rabbit, and mouse. In some embodiments, uABTs bind to IgG molecules and not human IgA or IgM.

Various TBTs can be utilized in accordance with the present disclosure. In attempts to discover effective cellular targets for cancer therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides, i.e. tumor-associated antigens (TAA), has given rise to the ability to specifically target cancer cells for destruction. A TBT that selectively binds a TAA is able to target a cancer cell of interest and enable a cell-specific recruitment of antibodies, e.g., endogenous antibodies, through the ABT. Additional suitable TBTs are provided in the present disclosure.

In some embodiments, the present disclosure provides compounds, and pharmaceutically acceptable compositions thereof, that are effective for recruiting antibodies to diseased cells, e.g., cancer cells. In some embodiments, provided compounds induce antibody-dependent effector functions. In some embodiments, provided compounds induce complement dependent cytotoxicity (CDC). In some embodiments, provided compounds induce direct cytotoxicity. In some embodiments, provided compounds inhibit biological functions associated with steric blockade. In some embodiments, provided compounds induce antibody-dependent cell-mediated virus inhibition (ADCVI). In some embodiments, provided compounds induce ADCC and kill cancer cells. In some embodiments, provided compounds induce ADCP and kill cancer cells. In some embodiments, provided compounds induce both ADCC and ADCP.

In some embodiments, the present disclosure provide an agent comprising:
an antibody binding moiety,
a target binding moiety, and
optionally a linker moiety,
   wherein the antibody binding moiety can bind to two or more antibodies which have different Fab regions.

In some embodiments, an antibody binding moiety, e.g., a universal antibody binding moiety, binds to an Fc region of an antibody. In some embodiments, an antibody binding moiety, e.g., a universal antibody binding moiety, binds to a conserved Fc region of an antibody. In some embodiments, an antibody binding moiety binds to an Fc region of an IgG antibody.

In some embodiments, the present disclosure provides compounds that have the general formula I:

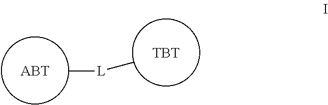

or a pharmaceutically acceptable salt thereof, wherein each variable is as defined and described herein. In some embodiments, a provided agent is a compound of formula I or a salt thereof.

In some embodiments, a provided agent is a compound of formula I-a or a salt thereof. In some embodiments, the present disclosure provides a compound of formula I-a:

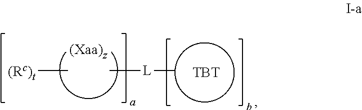

or a pharmaceutically acceptable salt thereof, wherein each variable is as defined and described in the present disclosure. In some embodiments, a provided compound of formula I is a compound of formula I-a.

In some embodiments, a provided agent is a compound of formula I-b or a salt thereof. In some embodiments, the present disclosure provides a compound of formula I-b:

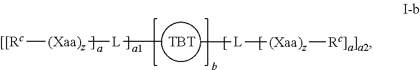

or a pharmaceutically acceptable salt thereof, wherein each variable is as defined and described in the present disclosure. In some embodiments, a provided compound of formula I is a compound of formula I-b.

In some embodiments, provided agents and compounds of the present disclosure, and pharmaceutically acceptable compositions thereof, are effective for recruiting antibodies to diseased cells, e.g., cancer cells. In some embodiments, the present disclosure provides compounds that have the general formula II:

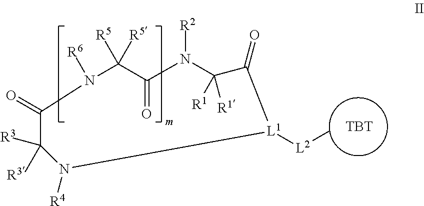

or a pharmaceutically acceptable salt thereof, wherein each variable is as defined and described herein. In some embodiments, a provided agent is a compound of formula II or a salt thereof. In some embodiments, a provided compound of formula I is a provided compound of formula II or a salt thereof. In some embodiments, a compound having the structure of formula I-a is a compound of formula II.

In some embodiments, the present disclosure provides compounds that have the general formula III:

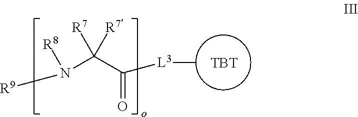

or a pharmaceutically acceptable salt thereof, wherein each variable is as defined and described herein. In some embodiments, a provided agent is a compound of formula III or a salt thereof. In some embodiments, a provided compound of formula I is a provided compound of formula III or a salt thereof. In some embodiments, a compound having the structure of formula I-b is a compound of formula III.

Compounds of the present disclosure, and pharmaceutically acceptable compositions thereof, are useful for treating a variety of diseases, disorders or conditions. Such diseases, disorders, or conditions include those described herein. In some embodiments, a condition, disorder or disease is cancer.

### DETAILED DESCRIPTION OF CERTAIN EMBODIMENTS

### 1. General Description of Certain Embodiments of the Invention

In some embodiments, the present disclosure provides ARM agents that comprise antibody binding moieties that can bind to antibodies with different Fab structures (“uABT”). Particularly, in some embodiments, the present disclosure provides agents that comprises antibody binding moieties that bind to Fc region of antibodies, and such binding to Fc regions of antibodies do not interfere one or more immune activities of the antibodies, e.g., interaction with Fc receptors (e.g., CD16a), recruitment of effector cells like NK cells for ADCC, macrophage for ADCP, etc. As those skilled in the art will appreciate, provided technologies (agents, compounds, compositions, methods, etc.) of the present disclosure comprising uABTs can provide various advantages, for example, provided technologies can utilize antibodies having various Fab regions in the immune system to avoid or minimize undesired effects of antibody variations among a patient population, and can trigger, and/or enhance, immune activities toward targets, e.g., killing target diseased cells such as cancer cells.

In some embodiments, technologies of the present disclosure are useful for recruiting antibodies to cancer cells. In some embodiments, provided technologies are useful for modulating immune activities, such as ADCC, ADCP, and combinations thereof against targets (diseased cells, foreign objects or entities, etc.). In some embodiments, provided technologies are useful for modulating ADCC against target cells, e.g., diseased cells such as cancer cells. In some embodiments, provided technologies are useful for modulating ADCP against target cells, e.g., diseased cells such as cancer cells. In some embodiments, provided agents can inhibit protein activities. In some embodiments, a target binding moiety is an inhibitor moiety. In some embodiments, a target binding moiety is an enzyme inhibitor moiety.

In some embodiments, the present disclosure provide an agent comprising:
an antibody binding moiety,
a target binding moiety, and
optionally a linker moiety,
   wherein the antibody binding moiety can bind to two or more antibodies which have different Fab regions.

In some embodiments, provided agents comprise two or more antibody binding moieties. In some embodiments, provided agents comprise two or more target binding moieties.

An antibody binding moiety may interact with any portion of an antibody. In some embodiments, an antibody binding moiety binds to an Fc region of an antibody. In some embodiments, an antibody binding moiety binds to a conserved Fc region of an antibody. In some embodiments, an antibody binding moiety binds to an Fc region of an IgG antibody. As appreciated by those skilled in the art, various antibody binding moieties, linkers, and target binding moieties can be utilized in accordance with the present disclosure. Among other things, as demonstrated in the Examples, in some embodiments, the present disclosure provides antibody binding moieties, linkers, and target binding moieties and combinations thereof that are particularly useful and effective for constructing ARM molecules to recruit antibodies to target cells, and/or to trigger, generate, encourage, and/or enhance immune system activities toward target cells, e.g., diseased cells such as cancer cells.

In some embodiments, the present disclosure provides antibody binding moieties and/or agents (e.g., compounds of various formulae described in the present disclosure, ARM molecules of the present disclosure, etc.) comprising antibody binding moieties that can bind to a Fc region that is bound to Fc receptors, e.g., FcγRIIIa, CD16a, etc. In some embodiments, provided moieties and/or agents comprising antibody binding moieties that bind to a complex comprising an Fc region and an Fc receptor. In some embodiments, the present disclosure provides a complex comprising:
an agent comprising:
   an antibody binding moiety,
   a target binding moiety, and
   optionally a linker moiety,
an Fc region, and
an Fc receptor,
   wherein the antibody binding moiety of the agent can bind to two or more antibodies which have different Fab regions.

In some embodiments, an Fc region is an Fc region of an endogenous antibody of a subject. In some embodiments, an Fc region is an Fc region of an exogenous antibody. In some embodiments, an Fc region is an Fc region of an administered agent. In some embodiments, an Fc receptor is of a diseased cell in a subject. In some embodiments, an Fc receptor is of a cancer cell in a subject.

In certain embodiments, the present invention provides a compound of formula I:

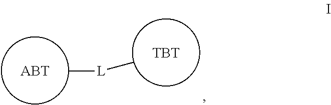

or a pharmaceutically acceptable salt thereof, wherein:
ABT is an antibody binding moiety;
L is a bivalent linker moiety that connects ABT with TBT; and
TBT is a target binding moiety.

In some embodiments, ABT is a universal antibody binding moiety.

In some embodiments, an antibody binding moiety comprises one or more amino acid residues. In some embodiments, an antibody binding moiety is or comprises a peptide moiety. In some embodiments, an antibody binding moiety is or comprises a cyclic peptide moiety. In some embodiments, such antibody binding moiety comprises one or more natural amino acid residues. In some embodiments, such antibody binding moiety comprises one or more unnatural natural amino acid residues.

In some embodiments, an amino acid has the structure of formula A-I:

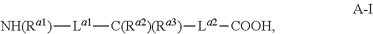

or a salt thereof, wherein:
each of R^{a1}, R^{a2}, R^{a3} is independently -L^{a}-R′;
each of L^{a1} and L^{a2} is independently L^{a};
each L^{a} is independently a covalent bond, or an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, an antibody-binding moiety is a cyclic peptide moiety. In some embodiments, the present disclosure provides a compound of formula I-a:

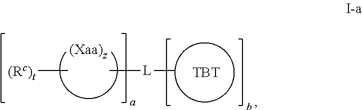

or a salt thereof, wherein:
each Xaa is independently an amino acid residue;
t is 0-50;
z is 1-50;
L is a linker moiety;
TBT is a target binding moiety;
each R^{c} is independently -L^{a}-R′;
each of a and b is independently 1-200;
each L^{a} is independently a covalent bond, or an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a is 1. In some embodiments, b is 1. In some embodiments, a is 1 and b is 1, and a compound of formula I-a has the structure of

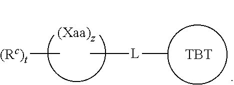

In some embodiments, each amino acid residue, e.g., each Xaa in formula I-a, is independently a residue of amino acid having the structure of formula A-I. In some embodiments, each Xaa independently has the structure of —N(R^{a1})-L^{a1}-C(R^{a2})(R^{a3})-L^{a2}-CO—. In some embodiments, two or more side chains of the amino acid residues, e.g., in compounds of formula I-a, (e.g., Ra or R^{a3} of one amino acid residue with Ra or R^{a3} of another amino acid residue) are optionally take together to form a bridge (e.g., compounds I-10, I-12, I-14, I-18, I-19, I-22, I-23, I-25, etc.), e.g., in some embodiments, two cysteine residues form a —S—S— bridge as typically observed in natural proteins. In some embodiments, a formed bridge has the structure of L^{b}, wherein L^{b} is L^{a} as described in the present disclosure. In some embodiments, each end of L^{b} independently connects to a backbone atom of a cyclic peptide (e.g., a ring atom of the ring formed by -(Xaa)z- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)z- in formula I-a through a side chain of an amino acid residue (e.g., Xaa in formula I-a). In some embodiments, such a side chain comprises an amino group or a carboxylic acid group.

In some embodiments,

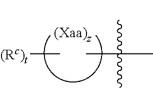

is an antibody binding moiety

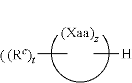

binds to an antibody). In some embodiments,

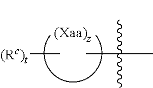

is a universal antibody binding moiety. In some embodiments,

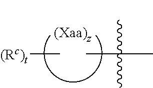

is a universal antibody binding moiety which can bind to antibodies having different Fab regions. In some embodiments,

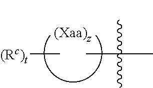

is a universal antibody binding moiety that can bind to a Fc region. In some embodiments, an antibody binding moiety, e.g., a universal antibody binding moiety having the structure of

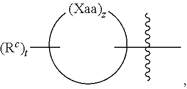

can bind to a Fc region bound to an Fc receptor. In some embodiments, an antibody binding moiety, e.g., of an antibody binding moiety having the structure of

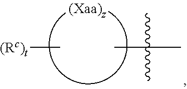

has the structure of

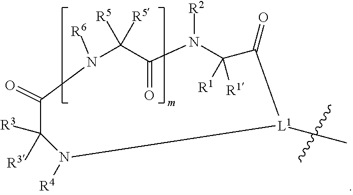

In some embodiments,

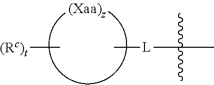

has the structure of

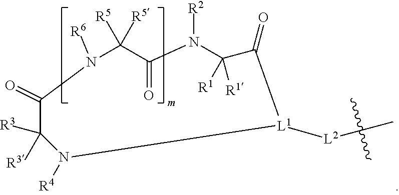

In certain embodiments, the present invention provides a compound of formula II:

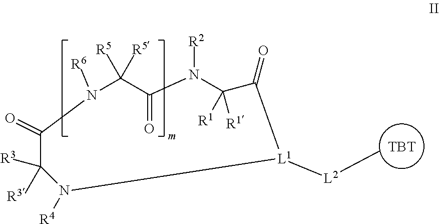

or a pharmaceutically acceptable salt thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   an R⁵ group and the R³ group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or
   two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R³ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;

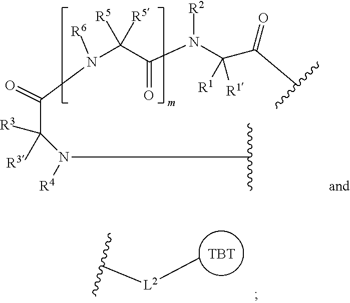

L¹ is a trivalent linker moiety that connects
L² is a covalent bond or a C₁₋₃₀ optionally substituted bivalent straight or branched saturated or unsaturated hydrocarbon chain wherein 1-10 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—,

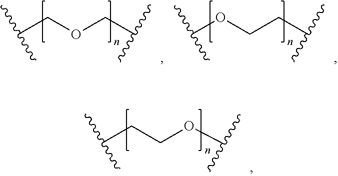

or -Cy¹-, wherein each -Cy¹- is independently a 5-6 membered heteroarylenyl with 1-4 heteroatoms independently selected from nitrogen, oxygen or sulfur;
TBT is a target binding moiety; and
each of m and n is independently 1, 2, 3, 4, 5, 6, 7, 8, 9, or 10.

In some embodiments, an antibody binding moiety is or comprises a peptide moiety. In some embodiments, the present disclosure provides a compound having the structure of formula I-b:

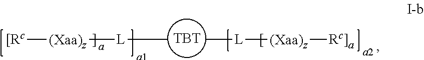

or a salt thereof, wherein:
   each Xaa is independently an amino acid residue;
   each z is independently 1-50;
   each L is independently a linker moiety;
   TBT is a target binding moiety,
   each R^{c} is independently -L^{a}-R′;
   each of a1 and a2 is independently 0 or 1, wherein at least one of a1 and a2 is not 0;
   each of a and b is independently 1-200;
   each L^{a} is independently a covalent bond, or an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
   each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
   each R′ is independently —R, —C(O)R, —CO₂R, or —SO₂R;
   each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
   two R groups are optionally and independently taken together to form a covalent bond, or:
   two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
   two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a1 is 1. In some embodiments, a2 is 1. In some embodiments, b is 1. In some embodiments, a compound of formula I-b has the structure of

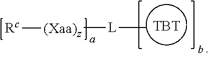

In some embodiments, a compound of formula I-b has the structure of

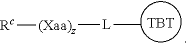

In some embodiments, a compound of formula I-b has the structure of

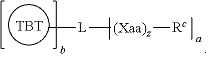

In some embodiments, a compound of formula I-b has the structure of

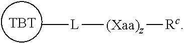

In some embodiments, each amino acid residue, e.g., each Xaa in formula I-b, is independently a residue of amino acid having the structure of formula A-I. In some embodiments, each Xaa independently has the structure of —N(R^{a1})-L^{a1}-C(R^{a2})(R^{a3})-L^{a2}-CO—. In some embodiments, two or more side chains of the amino acid residues, e.g., in compounds of formula I-a, (e.g., R^{a2} or R^{a3} of one amino acid residue with R^{a2} or R^{a3} of another amino acid residue) are optionally take together to form a bridge (e.g., compounds I-10, I-12, I-14, I-18, I-19, I-22, I-23, I-25, etc.), e.g., in some embodiments, two cysteine residues form a —S—S— bridge as typically observed in natural proteins. In some embodiments, a formed bridge has the structure of L^{b}, wherein L^{b} is L^{a} as described in the present disclosure. In some embodiments, each end of L^{v} independently connects to a backbone atom of a cyclic peptide (e.g., a ring atom of the ring formed by -(Xaa)_{z}- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)_{z}- in formula I-b through a side chain of an amino acid residue (e.g., Xaa in formula I-a). In some embodiments, such a side chain comprises an amino group or a carboxylic acid group.

In some embodiments, R^{c}-(Xaa)z- is an antibody binding moiety (R^{c}-(Xaa)z-H binds to an antibody). In some embodiments, R^{c}-(Xaa)z- is a universal antibody binding moiety. In some embodiments, R^{c}-(Xaa)z- is a universal antibody binding moiety which can bind to antibodies having different Fab regions. In some embodiments, R^{c}-(Xaa)z- is a universal antibody binding moiety that can bind to a Fc region. In some embodiments, an antibody binding moiety, e.g., a universal antibody binding moiety having the structure of R^{c}-(Xaa)z-, can bind to a Fc region which binds to an Fc receptor. In some embodiments, R^{c}-(Xaa)z- has the structure of

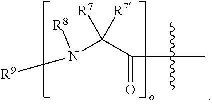

In some embodiments, R^{c}-(Xaa)z-L- has the structure of

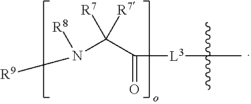

In certain embodiments, the present invention provides a compound of formula III:

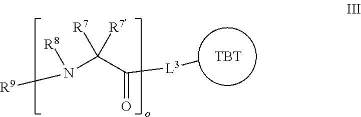

or a pharmaceutically acceptable salt thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered optionally substituted saturated or partially unsaturated spirocyclic carbocyclic ring or a 3-8 membered optionally substituted saturated or partially unsaturated spirocyclic heterocyclic ring having 1-2 heteroatoms independently selected from nitrogen, oxygen, or sulfur;
each of R⁷ is independently hydrogen or optionally substituted C₁₋₃ aliphatic;
each of R⁸ is independently hydrogen, or optionally substituted C₁₋₄ aliphatic, or:
   an R⁸ group and its adjacent R⁷ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
R⁹ is hydrogen, optionally substituted C₁₋₃ aliphatic, or —C(O)-(optionally substituted C₁₋₃ aliphatic);
L³ is a bivalent linker moiety that connects

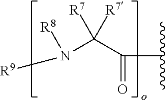

with TBT;
TBT is a target binding moiety; and
is 1, 2, 3, 4, 5, 6, 7, 8, 9, or 10.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

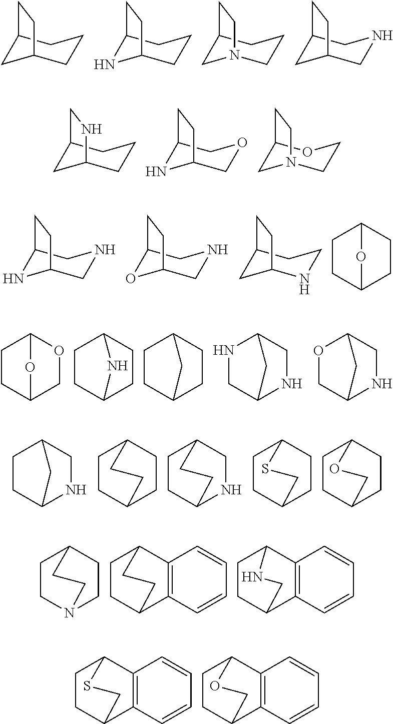

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR″ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

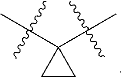 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —N(R^{∘})C(NR^{∘})N(R^{∘})₂; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O) C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —)N(R^{∘})S(O)₂NR^{∘}₂; —)N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—) N(R^{∘})₂; or —(C₁₋₄ straight or branched alkylene)C(O)O—N(R^{∘})₂, wherein each R^{c} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, —(CH₂)₀₋₂OH, -(haloR^{●}), —(CH₂)₀₋₂OR^{●}, —(CH₂)₀₋₂CH(OR^{●})₂; —O(haloR^{●}), —CN, —N₃, —(CH₂)₀₋₂C(O)R^{∘}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)⁰⁻¹Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR 2, ═NNHC(O)R*, —NNHC(O)OR*, ═NNHS(O)₂R*, ═NR*, ═NOR*, —O(C(R*₂))₂₋₃₀—, or —S(C(R*2))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O) OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O) C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of RT, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O) OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl) 4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, R^{x}, of a provided compound comprises one or more deuterium atoms.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

Tumor-associated cell surface antigen polypeptides, i.e. tumor-associated antigens (TAA), that allow the ability to specifically target cancer cells for destruction are listed below.

TAA include, but are not limited to: 5T4, AOC3, ALK, AXL, C242, CA-125, CCL11, CCR 5, CD2, CD3, CD4, CD5, CD15, CA15-3, CD18, CD19, CA19-9, CD20, CD22, CD23, CD25, CD28, CD30, CD31, CD33, CD37, CD38, CD40, CD41, CD44, CD44 v6, CD51, CD52, CD54, CD56, CD62E, CD62P, CD62L, CD70, CD74, CD79-B, CD80, CD125, CD138, CD141, CD147, CD152, CD154, CD326, CEA, CTLA-4, CXCR2, EGFR, ErbB2, ErbB3, EpCAM, EphA2, EphB2, EphB4, FGFR (i.e. FGFR1, FGFR2, FGFR3, FGFR4), FLT3, folate receptor, FAP, GD2, GD3, GPNMB, HGF, HER2, ICAM, IGF-1 receptor, VEGFR1, TRPV1, CFTR, gpNMB, CA9, Cripto, c-KIT, c-MET, ACE, APP, adrenergic receptor-beta2, Claudine 3, Mesothelin, MUC1, RON, ROR1, PD-L1, PD-L2, B7-H3, B7-B4, IL-2 receptor, IL-4 receptor, IL-13 receptor, integrins (including α4, α_{ν}β3, α_{ν}β5, α_{ν}β6, α₁β₄, α₄β₁, α₄β₇, α₅β₁, α₆β₄, α_{IIb}β₃ integrins), IFN-α, IFN-γ, IgE, IGF-1 receptor, IL-1, IL-12, IL-23, IL-13, IL-22, IL-4, IL-5, IL-6, interferon receptor, ITGB2 (CD18), LFA-1 (CD11a), L-selectin (CD62L), mucin, MUC1, myostatin, NCA-90, NGF, PDGFRa, phosphatidylserine, prostatic carcinoma cell, prostate-specific membrane antigen (PSMA), RANKL, Rhesus factor, SLAMF7, sphingosine-1-phosphate, TAG-72, T-cell receptor, tenascin C, TGF-1, TGF-β2, TGF-β, TNF-α, TRAIL-R1, TRAIL-R2, tumor antigen CTAA16.88, VEGFA, VEGFR2, vimentin, and the like.

In some embodiments, tumor-associated antigens are or comprise carbohydrates. In some embodiments, provided TBTs target such TAAs. In some embodiments, a carbohydrate is part of a glycoprotein. In some embodiments, a carbohydrate is part of a glycolipid. Many conditions, disorders and diseases, e.g., various types of cancers, are associated aberrant glycosylation. Tumor-associated carbohydrate antigens (TACAs) include and/or are associated with altered sialic acid expression, altered Lewis carbohydrate antigen expression, altered ganglioside expression, etc. TBTs of the present disclosure can target various types of TACAs, including those described in the art, e.g., in Chua and Durrant, Monoclonal Antibodies Against Tumour-Associated Carbohydrate Antigens, Carbohydrate Mahmut Caliskan, IntechOpen, DOI: 10.5772/66996.

Additionally, the TBT can be a high affinity binding moiety to one or more tumor-associated antigens or cell-surface receptors selected from (1)-(36):
(1) BMPR1B (bone morphogenetic protein receptor-type IB, Genbank accession no. NM.sub.--001203);
(2) E16 (LAT1, SLC7A5, Genbank accession no. NM.sub.--003486);
(3) STEAP1 (six transmembrane epithelial antigen of prostate, Genbank accession no. NM.sub.--012449);
(4) 0772P (CA125, MUC16, Genbank accession no. AF361486);
(5) MPF (MPF, MSLN, SMR, megakaryocyte potentiating factor, mesothelin, Genbank accession no. NM.sub.-005823);
(6) Napi3b (NAPI-3B, NPTIIb, SLC34A2, solute carrier family 34 (sodium phosphate), member 2, type II sodium-dependent phosphate transporter 3b, Genbank accession no. NM.sub.--006424);
(7) Sema 5b (FLJ10372, KIAA1445, Mm.42015, SEMA5B, SEMAG, Semaphorin 5b H log, sema domain, seven thrombospondin repeats (type 1 and type 1-like), transmembrane domain (TM) and short cytoplasmic domain, (semaphorin) 5B, Genbank accession no. AB040878);
(8) PSCA hlg (2700050C12Rik, C530008016Rik, RIKEN cDNA 2700050C12, RIKEN cDNA 2700050C12 gene, Genbank accession no. AY358628);
(9) ETBR (Endothelin type B receptor, Genbank accession no. AY275463);
(10) MSG783 (RNF124, hypothetical protein FLJ20315, Genbank accession no. NM.sub.--017763);
(11) STEAP2 (HGNC.sub.--8639, IPCA-1, PCANAP1, STAMPI, STEAP2, STMP, prostate cancer associated gene 1, prostate cancer associated protein 1, six transmembrane epithelial antigen of prostate 2, six transmembrane prostate protein, Genbank accession no. AF455138);
(12) TrpM4 (BR22450, FLJ20041, TRPM4, TRPM4B, transient receptor potential cation channel, subfamily M, member 4, Genbank accession no. NM.sub.--017636);
(13) CRIPTO (CR, CR1, CRGF, CRIPTO, TDGF1, teratocarcinoma-derived growth factor, Genbank accession no. NP.sub.--003203 or NM.sub.--003212);
(14) CD21 (CR2 (Complement receptor 2) or C3DR (C3d/Epstein Barr virus receptor) or Hs.73792 Genbank accession no. M26004);
(15) CD79b (CD79B, CD79.beta., IGb (immunoglobulin-associated beta), B29, Genbank accession no. NM.sub.--000626);
(16) FcRH2 (IFGP4, IRTA4, SPAP1A (SH2 domain containing phosphatase anchor protein 1a), SPAPIB, SPAP1C, Genbank accession no. NM.sub.--030764);
(17) HER2 (Genbank accession no. M1730);
(18) NCA (Genbank accession no. M18728);
(19) MDP (Genbank accession no. BC017023);
(20) IL20Rα (Genbank accession no. AF184971);
(21) Brevican (Genbank accession no. AF229053;
(22) EphB2R (Genbank accession no. NM.sub.--004442);
(23) ASLG659 (Genbank accession no. AX092328);
(24) PSCA (Genbank accession no. AJ297436);
(25) GEDA (Genbank accession no. AY260763;
(26) BAFF--R (B cell-activating factor receptor, BLyS receptor 3, BR3, NP.sub.--443177.1);
(27) CD22 (B-cell receptor CD22-B isoform, NP.sub.--001762.1);
(28) CD79a (CD79A, CD79.alpha., immunoglobulin-associated alpha, a B cell-specific protein that covalently interacts with Ig beta (CD79B) and forms a complex on the surface with Ig M molecules, transduces a signal involved in B-cell differentiation, Genbank accession No. NP.sub.--001774.1);
(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia, Genbank accession No. NP.sub.--001707.1);
(30) HLA-DOB (Beta subunit of MHC class II molecule (Ia antigen) that binds peptides and presents them to CD4+ T lymphocytes, Genbank accession No. NP.sub.--002111.1);
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability, Genbank accession No. NP.sub.--002552.2);
(32) CD72 (B-cell differentiation antigen CD72, Lyb-2, Genbank accession No. NP.sub.--001773.1);
(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis, Genbank accession No. NP.sub.--005573.1);
(34) FcRH1 (Fc receptor-like protein 1, a putative receptor for the immunoglobulin Fc domain that contains C2 type Ig-like and ITAM domains, may have a role in B-lymphocyte differentiation, Genbank accession No. NP.sub.--443170.1);
(35) IRTA2 (Immunoglobulin superfamily receptor translocation associated 2, a putative immunoreceptor with possible roles in B cell development and lymphomagenesis; deregulation of the gene by translocation occurs in some B cell malignancies, Genbank accession No. NP.sub.--112571.1); and
(36) TENB2 (putative transmembrane proteoglycan, related to the EGF/heregulin family of growth factors and follistatin, Genbank accession No. AF 179274.

### 3. Description of Exemplary Embodiments

In certain embodiments, the present invention provides a compound of formula I:

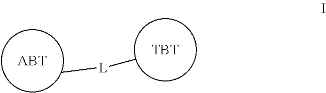

or a pharmaceutically acceptable salt thereof, wherein:
ABT is an antibody binding moiety;
L is a bivalent linker moiety that connects ABT with TBT; and
TBT is a target binding moiety.

In some embodiments, the present disclosure provides a compound of formula I-a or a salt thereof. In some embodiments, the present disclosure provides a compound of formula I-b or a salt thereof.

In certain embodiments, the present invention provides a compound of formula II,

or a pharmaceutically acceptable salt thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur;
   an R⁵ group and the R^{5′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; or
   two R⁵ groups are optionally taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R¹, R^{3′} and R^{5′} is independently hydrogen or C₁₋₃ aliphatic;
each of R², R⁴ and R⁶ is independently hydrogen, or C₁₋₄ aliphatic, or:
   R² and R¹ are optionally taken together with their intervening atoms to form a 4-8 membered saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered saturated or partially unsaturated monocyclic heterocyclic ring having 1-2 heteroatoms independently selected from nitrogen, oxygen, or sulfur;
L′ is a trivalent linker moiety that connects

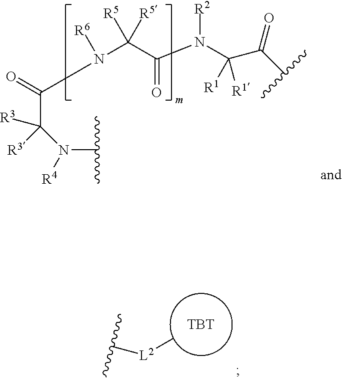

L² is a covalent bond or a C₁₋₁₀ bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—,

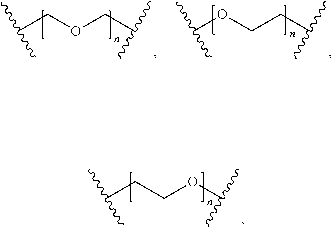

or -Cy¹-, wherein each -Cy¹- is independently a 5-6 membered heteroarylenyl with 1-4 heteroatoms independently selected from nitrogen, oxygen or sulfur;
TBT is a target binding moiety; and
each of m and n is independently 1, 2, 3, 4, 5, 6, 7, 8, 9, or 10.

In certain embodiments, the present invention provides a compound of formula III:

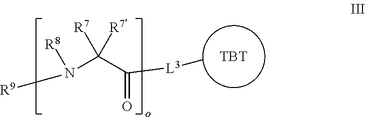

or a pharmaceutically acceptable salt thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur;
each of R^{7′} is independently hydrogen or C₁₋₃ aliphatic;
each of R⁸ is independently hydrogen, or C₁₋₄ aliphatic, or:
   an R⁸ group and its adjacent R⁷ group are optionally taken together with their intervening atoms to form a 4-8 membered saturated or partially unsaturated monocyclic heterocyclic ring having 1-2 heteroatoms independently selected from nitrogen, oxygen, or sulfur;
R⁹ is hydrogen, C₁₋₃ aliphatic, or —C(O) C₁₋₃ aliphatic;
L³ is a bivalent linker moiety that connects

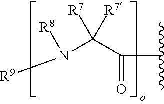

with TBT;
TBT is a target binding moiety; and
o is 1, 2, 3, 4, 5, 6, 7, 8, 9, or 10.

### Antibody Binding Moieties

Among other things, the present disclosure provides agents comprising universal antibody binding moieties which can bind to antibodies having different Fab regions and different specificity. In some embodiments, antibody binding moieties of the present disclosure are universal antibody binding moieties that bind to Fc regions. In some embodiments, binding of universal antibody binding moieties to Fc regions can happen at the same time as binding of Fc receptors, e.g., CD16a, to the same Fc regions (e.g., may at different locations/amino acid residues of the same Fc regions). In some embodiments, upon binding of universal antibody binding moieties, e.g., those in provided agents, compounds, methods, etc., an Fc region can still interact with Fc receptors and perform one or more or all of its immune activities, including recruitment of immune cells (e.g., effector cells such as NK cells), and/or triggering, generating, encouraging, and/or enhancing immune system activities toward target cells, tissues, objects and/or entities, for example, antibody-dependent cell-mediated cytotoxicity (ADCC) and/or ADCP.

Various universal antibody binding moieties can be utilized in accordance with the present disclosure. Among other things, the present disclosure provides technologies, e.g., those described in the Examples, for identifying and/or assessing universal antibody binding moieties and their utilization in ARMs. Those skilled in the art appreciates that additional technologies in the art may be suitable for identifying and/or assessing universal antibody binding moieties suitable for ARMs in accordance with the present disclosure. In some embodiments, a universal antibody binding moiety comprises one or more amino acid residues, each independently natural or unnatural. In some embodiments, a universal antibody binding moiety has the structure of

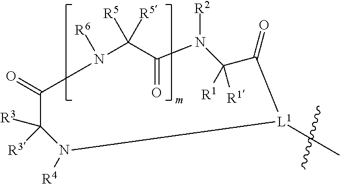

or salt form thereof. In some embodiments, a universal antibody binding moiety has the structure of

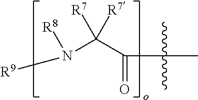

or salt form thereof. In some embodiments, a universal antibody binding moiety is or comprises a peptide moiety, e.g., a moiety having the structure of R^{c}-(Xaa)z-. In some embodiments, a universal antibody binding moiety is or comprises a cyclic peptide moiety, e.g., a moiety having the structure of

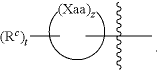

In some embodiments, a universal antibody binding moiety is R^{c}-(Xaa)z- or

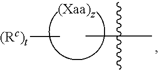

and is or comprises a peptide unit. In some embodiments, -(Xaa)z- is or comprises a peptide unit. In some embodiments, a peptide unit comprises an amino acid residue, e.g., a residue of an amino acid of formula A-I that has a positively charged side chain (e.g., at physiological pH about 7.4, “positively charged amino acid residue”, Xaa^{P}). In some embodiments, a peptide unit comprises R. In some embodiments, at least one Xaa is R. In some embodiments, a peptide unit is or comprises APAR. In some embodiments, a peptide unit is or comprises RAPA. In some embodiments, a peptide unit comprises an amino acid residue, e.g., a residue of an amino acid of formula A-I, that has a side chain comprising an aromatic group (“aromatic amino acid residue”, Xaa^{A}). In some embodiments, a peptide unit comprises a positively charged amino acid residue and an aromatic amino acid residue. In some embodiments, a peptide unit comprises W. In some embodiments, a peptide unit comprises a positively charged amino acid residue and an aromatic amino acid residue. In some embodiments, a peptide unit is or comprises Xaa^{A}XaaXaa^{P}Xaa^{P}. In some embodiments, a peptide unit is or comprises Xaa^{P}Xaa^{P}XaaXaa^{A}. In some embodiments, a peptide unit is or comprises Xaa^{P}Xaa^{A}Xaa^{P}. In some embodiments, a peptide unit is or comprises two or more Xaa^{P}Xaa^{A}Xaa^{P}. In some embodiments, a peptide unit is or comprises Xaa^{P}Xaa^{A}Xaa^{P}XaaXaa^{P}Xaa^{A}Xaa^{P}. In some embodiments, a peptide unit is or comprises Xaa^{P}Xaa^{P}Xaa^{A}Xaa^{A}Xaa^{P}. In some embodiments, a peptide unit is or comprises Xaa^{P}Xaa^{A}Xaa^{P}Xaa^{A}. In some embodiments, a peptide unit is or comprises two or more Xaa^{A}Xaa^{A}Xaa^{P}. In some embodiments, a peptide residue comprises one or more proline residues. In some embodiments, a peptide unit is or comprises HWRGWA. In some embodiments, a peptide unit is or comprises WGRR. In some embodiments, a peptide unit is or comprises RRGW. In some embodiments, a peptide unit is or comprises NRFRGKYK. In some embodiments, a peptide unit is or comprises NARKFYK. In some embodiments, a peptide unit comprises a positively charged amino acid residue, an aromatic amino acid residue, and an amino acid residue, e.g., a residue of an amino acid of formula A-I, that has a negatively charged side chain (e.g., at physiological pH about 7.4, “negatively charged amino acid residue”, Xaa^{N}). In some embodiments, a peptide residue is RHRENKD. In some embodiments, a peptide unit is TY. In some embodiments, a peptide unit is TYK. In some embodiments, a peptide unit is RTY. In some embodiments, a peptide unit is RTYK. In some embodiments, a peptide unit is or comprises a sequence selected from PAM. In some embodiments, a peptide unit is WHL. In some embodiments, a peptide unit is ELVW. In some embodiments, a peptide unit is or comprises a sequence selected from AWHLGELVW. In some embodiments, a peptide unit is or comprises a sequence selected from DCAWHLGELVWCT, which the two cysteine residues can form a disulfide bond as found in natural proteins. In some embodiments, a peptide unit is or comprises a sequence selected from Fc-III. In some embodiments, a peptide unit is or comprises a sequence selected from DpLpAWHLGELVW. In some embodiments, a peptide unit is or comprises a sequence selected from FcBP-1. In some embodiments, a peptide unit is or comprises a sequence selected from DpLpDCAWHLGELVWCT. In some embodiments, a peptide unit is or comprises a sequence selected from FcBP-2. In some embodiments, a peptide unit is or comprises a sequence selected from CDCAWHLGELVWCTC, wherein the first and the last cysteines, and the two cysteines in the middle of the sequence, can each independently form a disulfide bond as in natural proteins. In some embodiments, a peptide unit is or comprises a sequence selected from Fc-III-4c. In some embodiments, a peptide unit is or comprises a sequence selected from FcRM. In some embodiments, a peptide unit is or comprises a cyclic peptide unit. In some embodiments, a cyclic peptide unit comprises amide group formed by an amino group of a side chain and the C-terminus-COOH.

In some embodiments, -(Xaa)z- is or comprises [X¹]ₚ₁[X²]ₚ₂—X³X⁴X⁵X⁶X⁷X⁸X⁹X¹⁰X¹¹X¹²—[X¹³]ₚ₁₃—[X¹⁴]ₚ₁₄[X¹⁵]ₚ₁₅[X¹⁶]ₚ₁₆, wherein each of X¹, X², X³, X⁴, X⁵, X⁶, X⁷, X⁸, X⁹, X¹⁰, X¹¹, X¹², and X¹³ is independently an amino acid residue, e.g., of an amino acid of formula A-I, and each of p1, p2, p13, p14, p15 and p16 is independently 0, 1, 2, 3, 4, 5, 6, 7, 8, 9, or 10. In some embodiments, each of X¹, X², X³, X⁴, X⁵, X⁶, X⁷, X⁸, X⁹, X¹⁰, X¹¹, X¹², and X¹³ is independently an amino acid residue of an amino acid of formula A-I. In some embodiments, each of X¹, X², X³, X⁴, X⁵, X⁶, X⁷, X⁸, X⁹, X¹⁰, X¹¹, X¹², and X¹³ is independently a natural amino acid residue. In some embodiments, one or more of X¹, X², X³, X⁴, X⁵, X⁶, X⁷, X⁸, X⁹, X¹⁰, X¹¹, X¹², and X¹³ are independently an unnatural amino acid residue as described in the present disclosure.

In some embodiments, a peptide unit comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see compounds in Table 1). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises -Cy-. In some embodiments, L^{a} comprises -Cy-, wherein -Cy- is optionally substituted heteroaryl. In some embodiments, -Cy- is

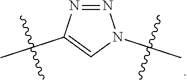 [SMILES: *c1cn(*)nn1]

In some embodiments, L^{a} is

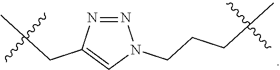 [SMILES: *CCCn1cc(C*)nn1]

In some embodiments, such an L^{a} can be formed by a —N₃ group of the side chain of one amino acid residue, and the = of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in I-27). In some embodiments, L^{a} is —(CH₂)₂—N(R′)—CO—(CH₂)₂—. In some embodiments, -Cy- is optionally substituted phenylene. In some embodiments, -Cy- is optionally substituted 1,2-phenylene. In some embodiments, L^{a} is

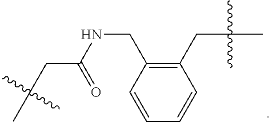 [SMILES: *CC(=O)NCc1ccccc1C*]

In some embodiments, L^{a} is

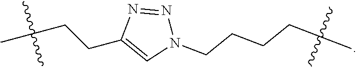 [SMILES: *CCCCn1cc(CC*)nn1]

In some embodiments, L^{a} is optionally substituted bivalent C₂₋₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, each of p1, p2, p13, p14, p15 and p16 is 0. In some embodiments, -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and
X¹² is Xaa^{A} or Xaa^{P}.
   In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, X⁵ is Xaa^{A} or Xaa^{P}. In some embodiments, X⁵ is Xaa^{A}. In some embodiments, X⁵ is Xaa^{P}. In some embodiments, X⁵ is an amino acid residue whose side chain comprises an optionally substituted saturated, partially saturated or aromatic ring. In some embodiments, X⁵ is

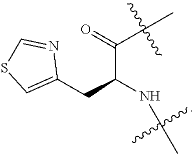 [SMILES: *N[C@@H](Cc1cscn1)C(*)=O]

In some embodiments, X⁵ is

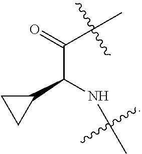 [SMILES: *N[C@H](C(*)=O)C1CC1]

In some embodiments, X⁶ is Xaa^{A}. In some embodiments, X⁶ is Xaa^{P}. In some embodiments, X⁶ is His. In some embodiments, X¹² is Xaa^{A}. In some embodiments, X¹² is Xaa^{P}. In some embodiments, X⁹ is Asp. In some embodiments, X⁹ is Glu. In some embodiments, X¹² is

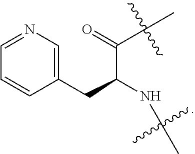 [SMILES: *N[C@@H](Cc1cccnc1)C(*)=O]

In some embodiments, X¹² is

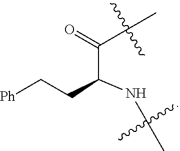 [SMILES: *N[C@@H](CCc1ccccc1)C(*)=O]

In some embodiments, each of X⁷, X¹⁰, and X¹¹ is independently an amino acid residue with a hydrophobic side chain (“hydrophobic amino acid residue”, Xaa^{H}). In some embodiments, X⁷ is Xaa^{H}. In some embodiments, X⁷ is

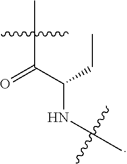 [SMILES: *N[C@@H](CC)C(*)=O]

In some embodiments, X⁷ is Val. In some embodiments, X¹⁰ is Xaa^{H}. In some embodiments, X¹⁰ is Met. In some embodiments, X¹⁰ is

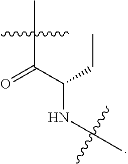 [SMILES: *N[C@@H](CC)C(*)=O]

In some embodiments, X¹¹ is Xaa^{H}. In some embodiments, X¹¹ is

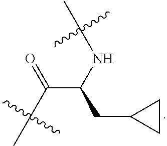 [SMILES: *N[C@@H](CC1CC1)C(*)=O]

In some embodiments, X⁸ is Gly. In some embodiments, X⁴ is Pro. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety.

In some embodiments, -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N}, and
X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, X⁵ and X¹⁰ are connected by L^{b}. In some embodiments, there is one linkage L^{b}. In some embodiments, X⁶ is Xaa^{A}. In some embodiments, X⁶ is Xaa^{P}. In some embodiments, X⁶ is His. In some embodiments, X⁹ is Asp. In some embodiments, X⁹ is Glu. In some embodiments, X¹² is Xaa^{A}. In some embodiments, X¹² is

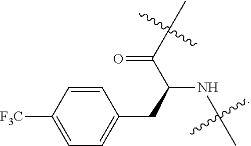 [SMILES: *N[C@@H](Cc1ccc(C(F)(F)F)cc1)C(*)=O]

In some embodiments, X¹² is

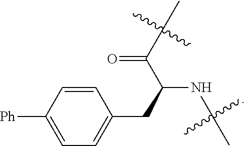 [SMILES: *N[C@@H](Cc1ccc(-c2ccccc2)cc1)C(*)=O]

In some embodiments, X¹² is

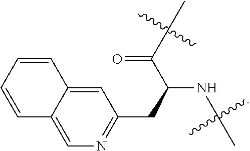 [SMILES: *N[C@@H](Cc1cc2ccccc2cn1)C(*)=O]

In some embodiments, each of X⁴, X⁷, and X¹¹ is independently Xaa^{H}. In some embodiments, X⁴ is Xaa^{H}. In some embodiments, X⁴ is Ala. In some embodiments, X⁷ is Xaa^{H}. In some embodiments, X⁷ is

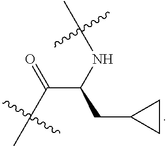 [SMILES: *N[C@@H](CC1CC1)C(*)=O]

In some embodiments, X¹¹ is Xaa^{H}. In some embodiments, X¹¹ is

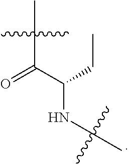 [SMILES: *N[C@@H](CC)C(*)=O]

In some embodiments, X⁸ is Gly. In some embodiments, X³ is Lys. In some embodiments, the —COOH of X¹² forms an amide bond with the side chain amino group of Lys (X³), and the other amino group of the Lys (X³) is connected to a linker moiety and then a target binding moiety. In some embodiments, L^{b} is

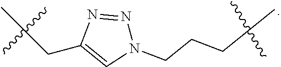 [SMILES: *CCCn1cc(C*)nn1]

In some embodiments, L^{b} is

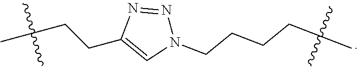 [SMILES: *CCCCn1cc(CC*)nn1]

In some embodiments, L^{b} connects two alpha-carbon atoms of two different amino acid residues. In some embodiments, both X⁵ and X¹⁰ are Cys, and the two —SH groups of their side chains form —S—S— (L^{b} is —CH₂—S—S—CH₂—).

In some embodiments, -(Xaa)z- is or comprises —X²X³X⁴X⁵X⁶X⁷X⁸X⁹X¹⁰X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L′;
L^{b} is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L′ is bonded to a backbone atom of one amino acid residue and a backbone atom of another amino acid residue, and comprises no backbone atoms;
X⁴ is Xaa^{A};
X⁵ is Xaa^{A} or Xaa^{P};
X⁸ is Xaa^{N}, and
X¹¹ is Xaa^{A}.
   In some embodiments, each of X², X³, X¹, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, X² and X¹² are connected by L^{b}. In some embodiments, L^{b} is —CH₂—S—S—CH₂—. In some embodiments, L^{b} is —CH₂—CH₂—S—CH₂—. In some embodiments, L^{b} is

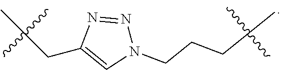 [SMILES: *CCCn1cc(C*)nn1]

In some embodiments, L^{b} is

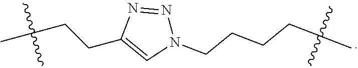 [SMILES: *CCCCn1cc(CC*)nn1]

In some embodiments, L^{b} is —CH₂CH₂CO—N(R′)—CH₂CH₂—. In some embodiments, R′ are taken together with an R group on the backbone atom that —N(R′)—CH₂CH₂— is bonded to to form a ring, e.g., as in I-27. In some embodiments, a formed ring is 3-, 4-, 5-, 6-, 7- or 8-membered. In some embodiments, a formed ring is monocyclic. In some embodiments, a formed ring is saturated. In some embodiments, L^{b} is

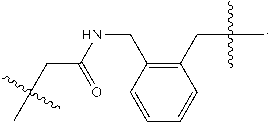 [SMILES: *CC(=O)NCc1ccccc1C*]

In some embodiments, L^{b} connects two alpha-carbon atoms of two different amino acid residues. In some embodiments, X⁴ is Xaa^{A}. In some embodiments, X⁴ is Tyr. In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. X¹¹ is Tyr. In some embodiments, both X² and X¹² are Cys, and the two —SH groups of their side chains form —S—S— (L^{b} is —CH₂—S—S—CH₂—). In some embodiments, each of X³, X⁶, X⁹, and X¹⁰ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁹ is Xaa^{H}. In some embodiments, X⁹ is Leu. In some embodiments, X⁹ is

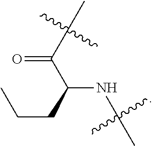 [SMILES: *N[C@@H](CCC)C(*)=O]

In some embodiments, X¹⁰ is Xaa^{H}. In some embodiments, X¹⁰ is Val. In some embodiments, X¹⁰ is

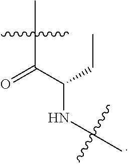 [SMILES: *N[C@@H](CC)C(*)=O]

In some embodiments, X⁷ is Gly. In some embodiments, p1 is 1. In some embodiments, X¹ is Asp. In some embodiments, p13 is 1. In some embodiments, p14, p15 and p16 are 0. In some embodiments, X¹³ is an amino acid residue comprising a polar uncharged side chain (e.g., at physiological pH, “polar uncharged amino acid residue”, Xaa^{L}). In some embodiments, X¹³ is Val. In some embodiments, p13 is 0. In some embodiments, R^{c} is —NHCH₂CH(OH)CH₃. In some embodiments, R^{c} is (R)—NHCH₂CH(OH)CH₃. In some embodiments, R^{c} is (S)—NHCH₂CH(OH)CH₃.

In some embodiments, -(Xaa)z- is or comprises —X²X³X⁴X⁵X⁶X⁷X⁸X⁹X¹⁰X¹¹X¹²— wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L′;
L^{b} is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L′ is bonded to a backbone atom of one amino acid residue and a backbone atom of another amino acid residue, and comprises no backbone atoms;
X⁵ is Xaa^{A} or Xaa^{P};
X⁸ is Xaa^{N}; and
X¹¹ is Xaa^{A}.
   In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² and X¹² are connected by L^{b}. In some embodiments, X⁴ and X⁹ are connected by L^{b}. In some embodiments, X⁴ and X¹⁰ are connected by L^{b}. In some embodiments, L^{b} is —CH₂—S—S—CH₂—. In some embodiments, L^{b} is

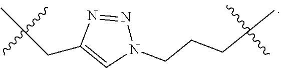 [SMILES: *CCCn1cc(C*)nn1]

In some embodiments, L^{b} is

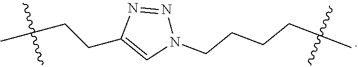 [SMILES: *CCCCn1cc(CC*)nn1]

In some embodiments, both X² and X¹² are Cys, and the two —SH groups of their side chains form —S—S— (L^{b} is —CH₂—S—S—CH₂—). In some embodiments, both X⁴ and X¹⁰ are Cys, and the two —SH groups of their side chains form —S—S-(L^{b} is —CH₂—S—S—CH₂—). In some embodiments, X⁴ and X⁹ are connected by L^{b}, wherein L^{b} is

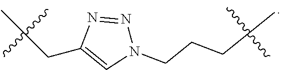 [SMILES: *CCCn1cc(C*)nn1]

In some embodiments, X⁴ and X⁹ are connected by L^{b}, wherein L^{b} is

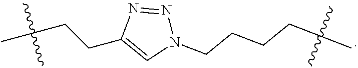 [SMILES: *CCCCn1cc(CC*)nn1]

In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. In some embodiments, X¹ is Tyr. In some embodiments, X¹¹ is

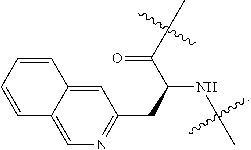 [SMILES: *N[C@@H](Cc1cc2ccccc2cn1)C(*)=O]

In some embodiments, X² and X¹² are connected by L^{b}, wherein L^{b} is —CH₂—S—CH₂CH₂—. In some embodiments, L^{b} connects two alpha-carbon atoms of two different amino acid residues. In some embodiments, each of X³, X⁶, and X⁹ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁶ is

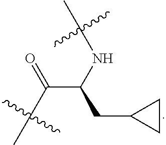 [SMILES: *N[C@@H](CC1CC1)C(*)=O]

In some embodiments, X⁹ is Xaa^{H}. In some embodiments, X⁹ is Leu. In some embodiments, X⁹ is

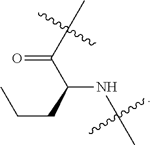 [SMILES: *N[C@@H](CCC)C(*)=O]

In some embodiments, X¹⁰ is Xaa^{H}. In some embodiments, X¹⁰ is Val. In some embodiments, X¹ is Gly. In some embodiments, p1 is 1. In some embodiments, X¹ is Xaa^{N}. In some embodiments, X¹ is Asp. In some embodiments, X¹ is Glu. In some embodiments, p13 is 1. In some embodiments, p14, p15 and p16 are 0. In some embodiments, X¹³ is Xaa^{L}. In some embodiments, X¹³ is Val. In some embodiments, -(Xaa)z- is or comprises —X²X³X⁴X⁵X⁶X⁷X⁸X⁹X¹⁰X¹¹X¹²X¹³X¹⁴X¹⁵X¹⁶—, wherein:
each of X², X³, X⁴, X⁵, X⁶, X⁷, X⁸, X⁹, X¹⁰, X¹¹, X¹², X¹³, X¹⁴, X¹⁵, and X¹⁶ is independently an amino acid residue;
at least two amino acid residues are connected through a linkage L^{b};
L^{b} is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L′ is bonded to a backbone atom of one amino acid residue and a backbone atom of another amino acid residue, and comprises no backbone atoms;

X³ is Xaa^{N};

X⁶ is Xaa^{A};

X⁷ is Xaa^{A} or Xaa^{P};

X⁹ is Xaa^{N}; and

X¹³ is Xaa^{A}.

In some embodiments, each of X², X³, X⁴, X³, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² are connected to X¹⁶ by L^{b}. In some embodiments, X⁴ are connected to X¹⁴ by L^{b}. In some embodiments, both X² and X¹⁶ are Cys, and the two —SH groups of their side chains form —S—S— (L^{b} is —CH₂—S—S—CH₂—). In some embodiments, both X⁴ and X¹⁴ are Cys, and the two —SH groups of their side chains form —S—S— (L^{b} is —CH₂—S—S—CH₂—). In some embodiments, L′ connects two alpha-carbon atoms of two different amino acid residues. In some embodiments, X³ is Asp. In some embodiments, X³ is Glu. In some embodiments, X⁵ is Xaa^{H}. In some embodiments, X⁵ is Ala. In some embodiments, X⁶ is Xaa^{A}. In some embodiments, X⁶ is Tyr. In some embodiments, X¹ is Xaa^{A}. In some embodiments, X⁷ is Xaa^{P}. In some embodiments, X⁷ is His. In some embodiments, X⁸ is Xaa^{H}. In some embodiments, X⁸ is Ala. In some embodiments, X⁹ is Gly. In some embodiments, X¹⁰ is Asp. In some embodiments, X¹⁰ is Glu. In some embodiments, X¹¹ is Xaa^{H}. In some embodiments, X¹¹ is Leu. In some embodiments, X¹² is Xaa^{H}. In some embodiments, X¹² is Val. In some embodiments, X¹³ is Xaa^{A}. In some embodiments, X¹³ is Tyr. In some embodiments, X¹⁵ is an amino acid residue comprising a polar uncharged side chain (e.g., at physiological pH, “polar uncharged amino acid residue”, Xaa^{L}). In some embodiments, X¹⁵ is Val. In some embodiments, p1 is 1. In some embodiments, In some embodiments, X¹ is Xaa^{N}. In some embodiments, X¹ is Asp. In some embodiments, X¹ is Glu.

As appreciated by those skilled in the art, an amino acid residue may be replaced by another amino acid residue having similar properties, e.g., one Xaa^{H} (e.g., Val, Leu, etc.) may be replaced with another Xaa^{H} (e.g., Leu, Ile, Ala, etc.), one Xaa^{A} may be replaced with another Xaa^{A}, one Xaa^{P} may be replaced with another Xaa^{P}, one Xaa^{N} may be replaced with another Xaa^{N}, one Xaa^{L} may be replaced with another Xaa^{L}, etc.

In some embodiments, an antibody binding moiety, e.g., a universal antibody binding moiety, is a universal antibody binding moiety of a compound in Table 1. In some embodiments, an antibody binding moiety, e.g., a universal antibody binding moiety, is or comprises optionally substituted

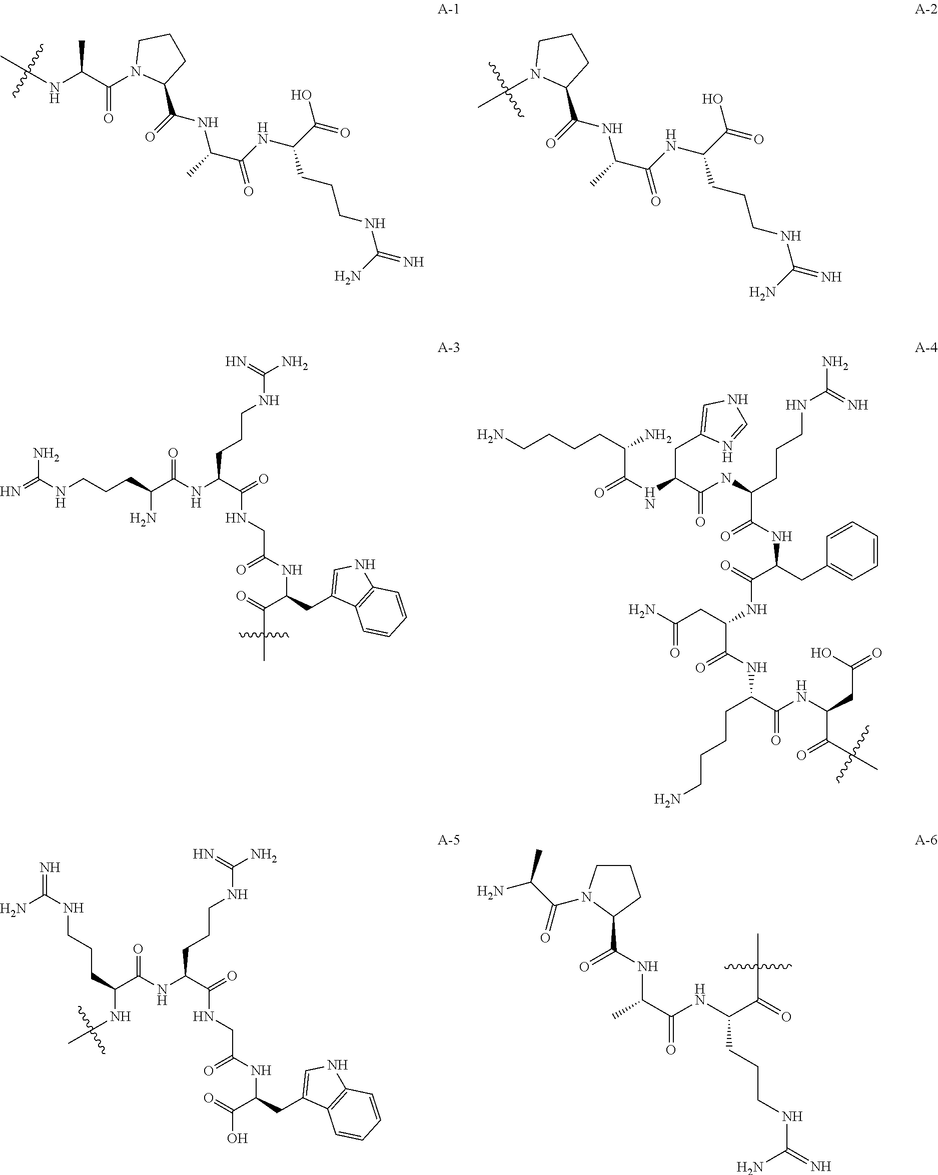

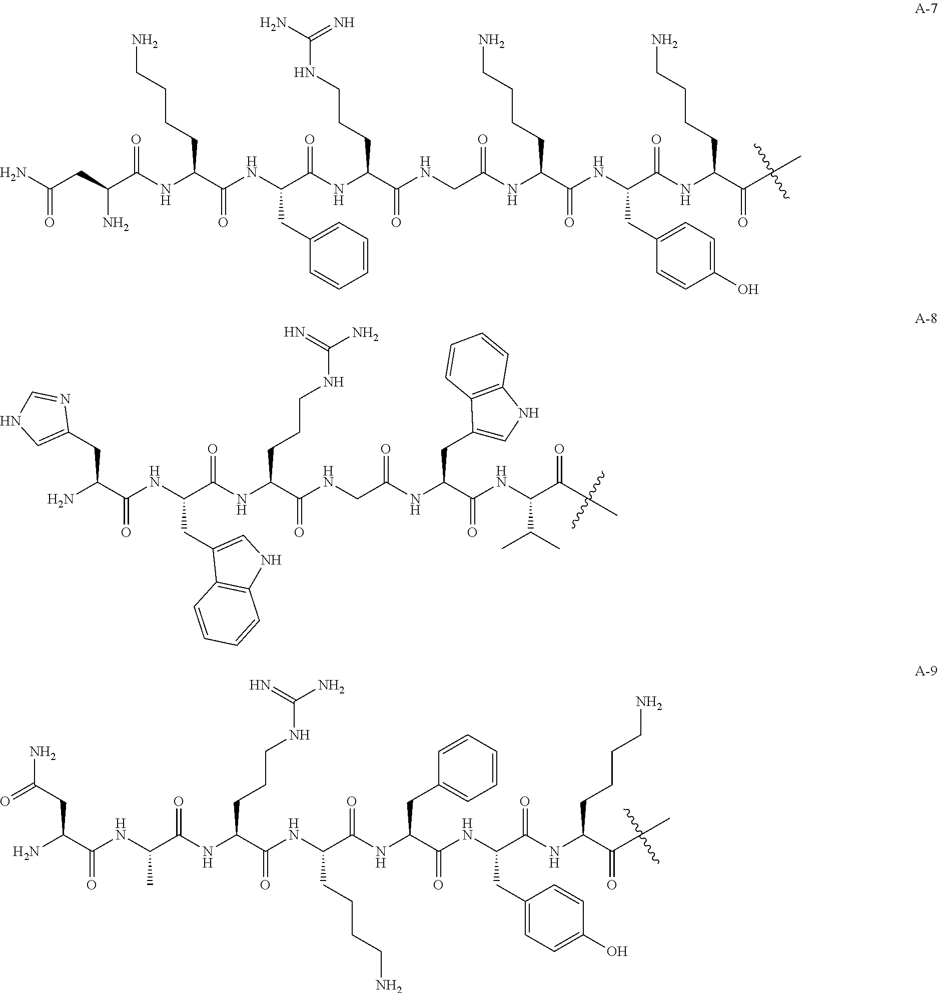 [SMILES: *C(=O)[C@H](CCCCN)NC(=O)[C@H](Cc1ccc(O)cc1)NC(=O)[C@H](CCCCN)NC(=O)CNC(=O)[C@H](CCCNC(=N)N)NC(=O)[C@H](Cc1ccccc1)NC(=O)[C@H](CCCCN)NC(=O)[C@@H](N)CC(N)=O ; *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@@H](N)Cc1c[nH]cn1)C(C)C ; *C(=O)[C@H](CCCCN)NC(=O)[C@H](Cc1ccc(O)cc1)NC(=O)[C@H](Cc1ccccc1)NC(=O)[C@H](CCCCN)NC(=O)[C@H](CCCNC(=N)N)NC(=O)[C@H](C)NC(=O)[C@@H](N)CC(N)=O]

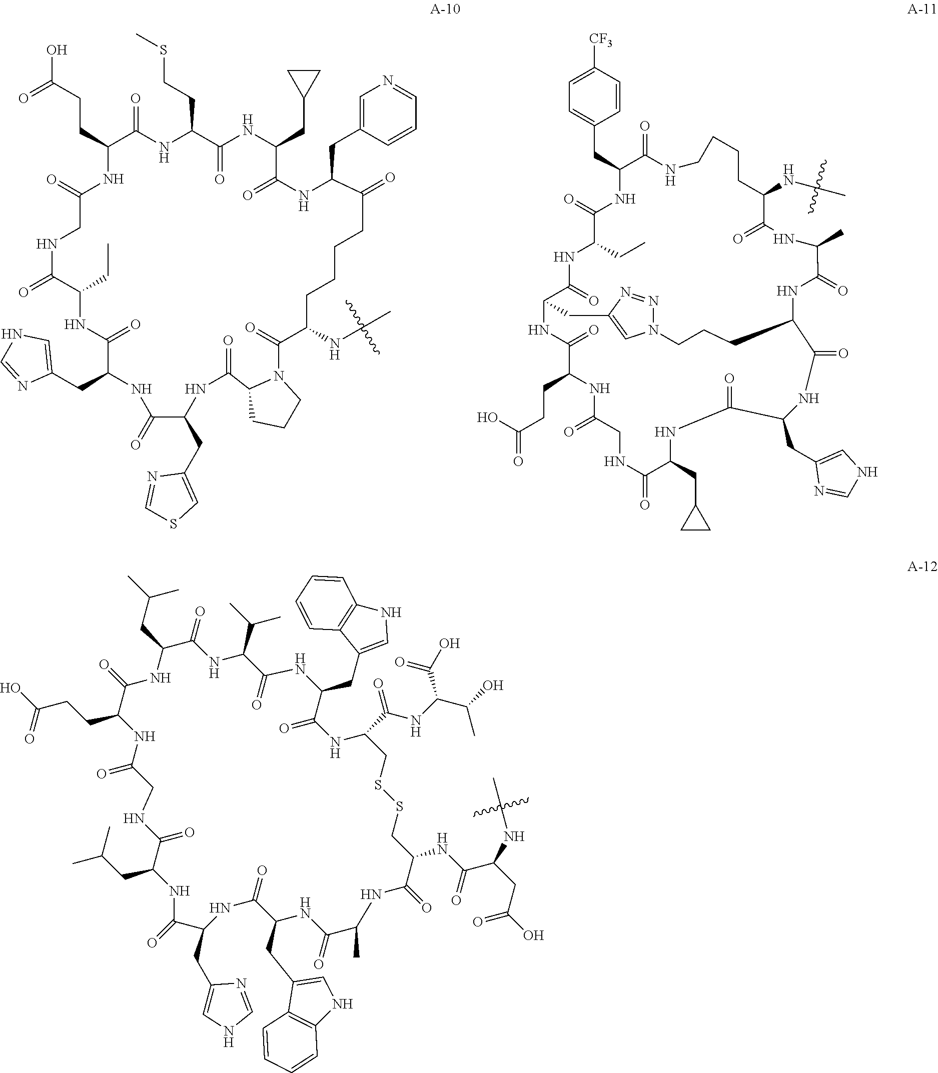

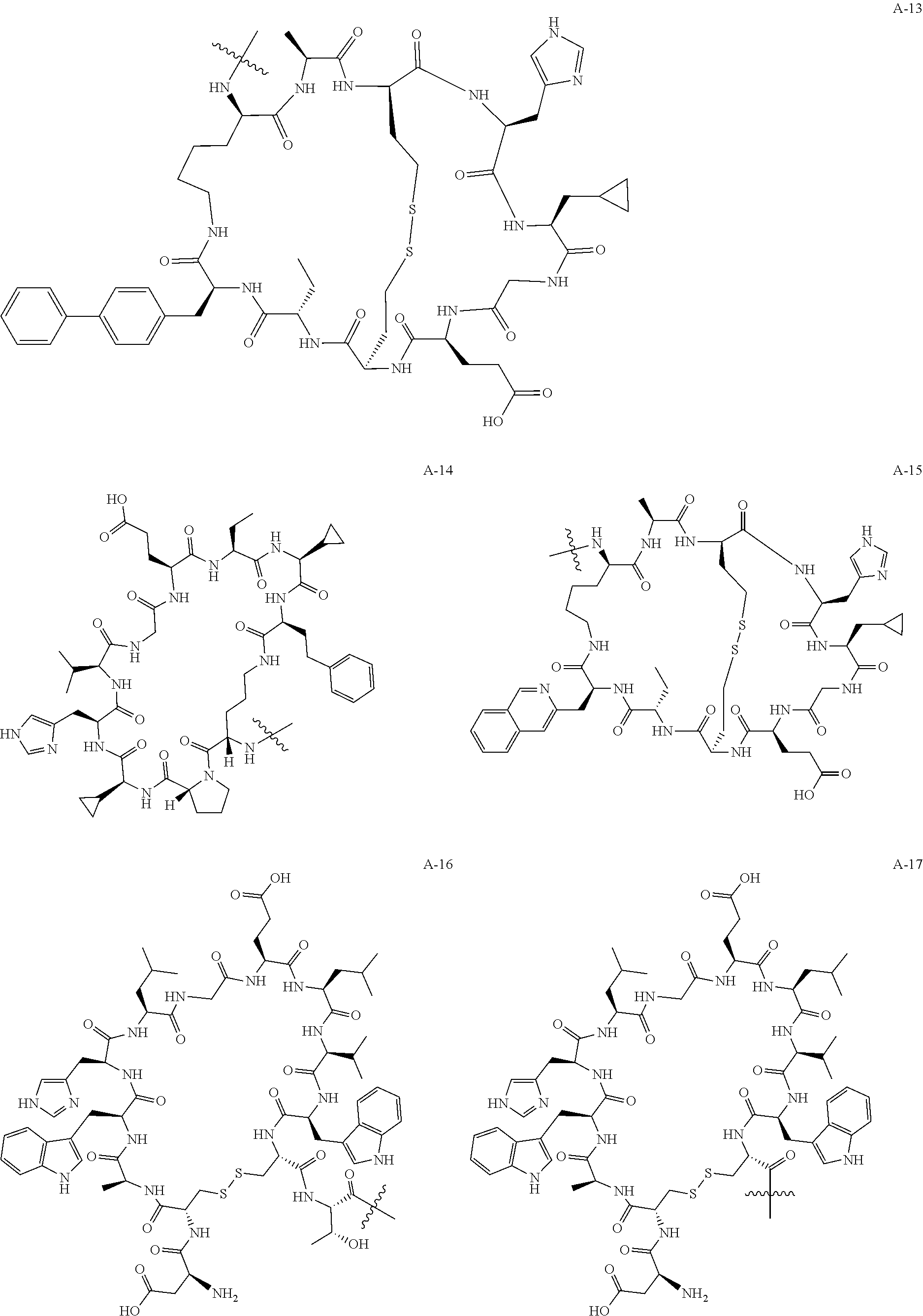

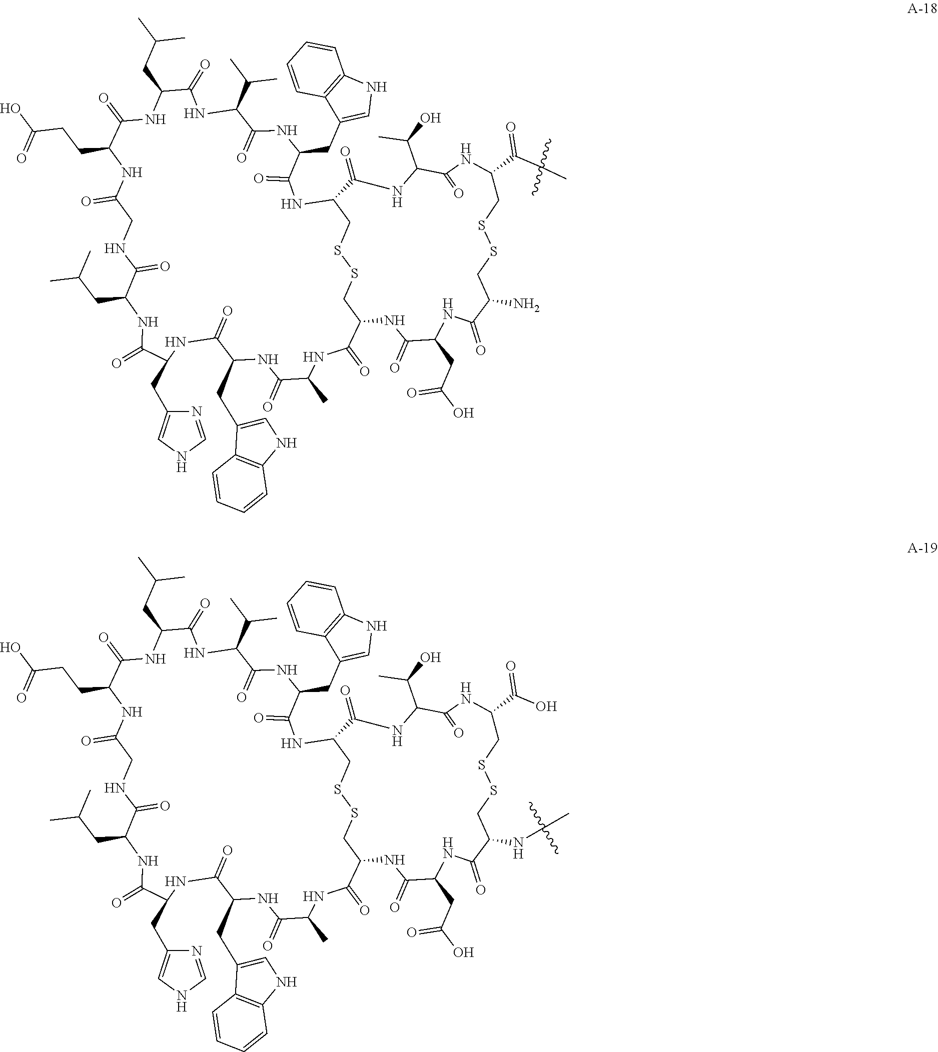

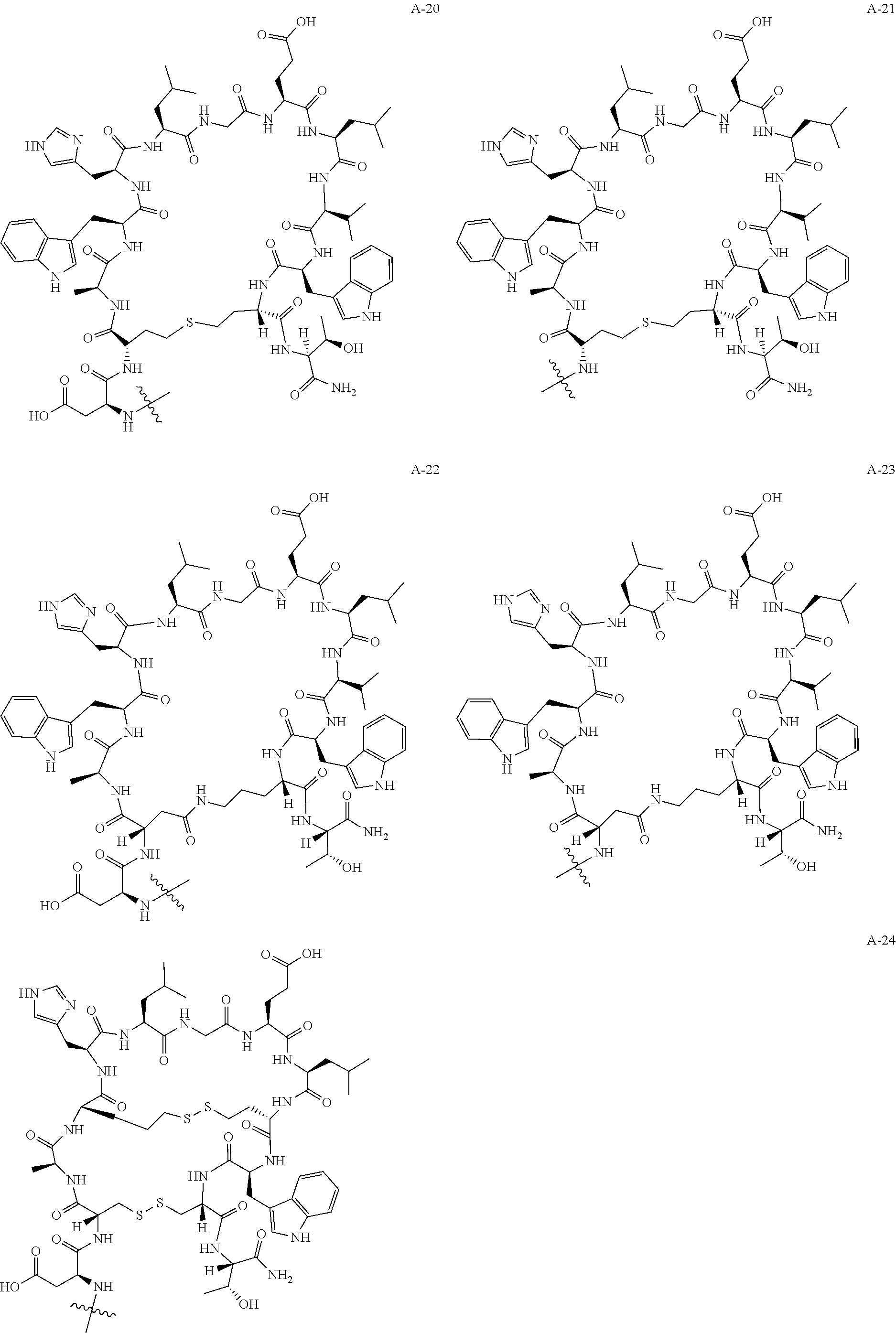

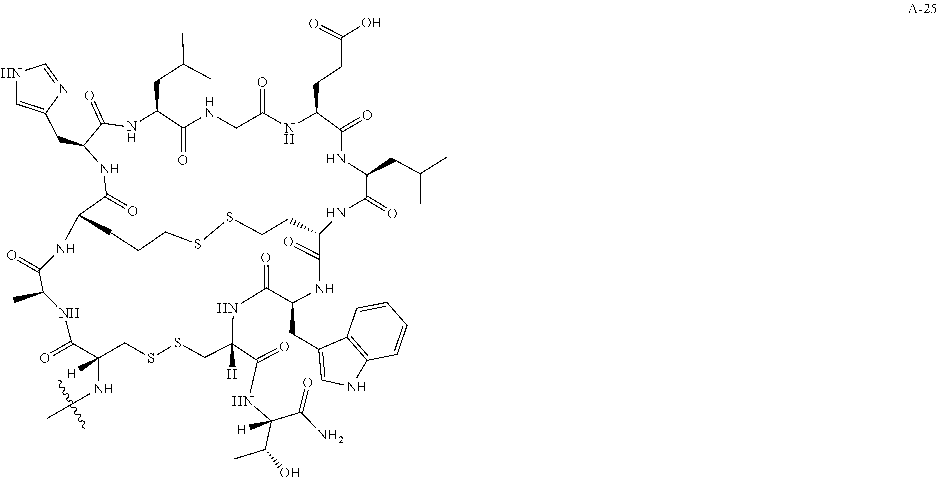 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2]

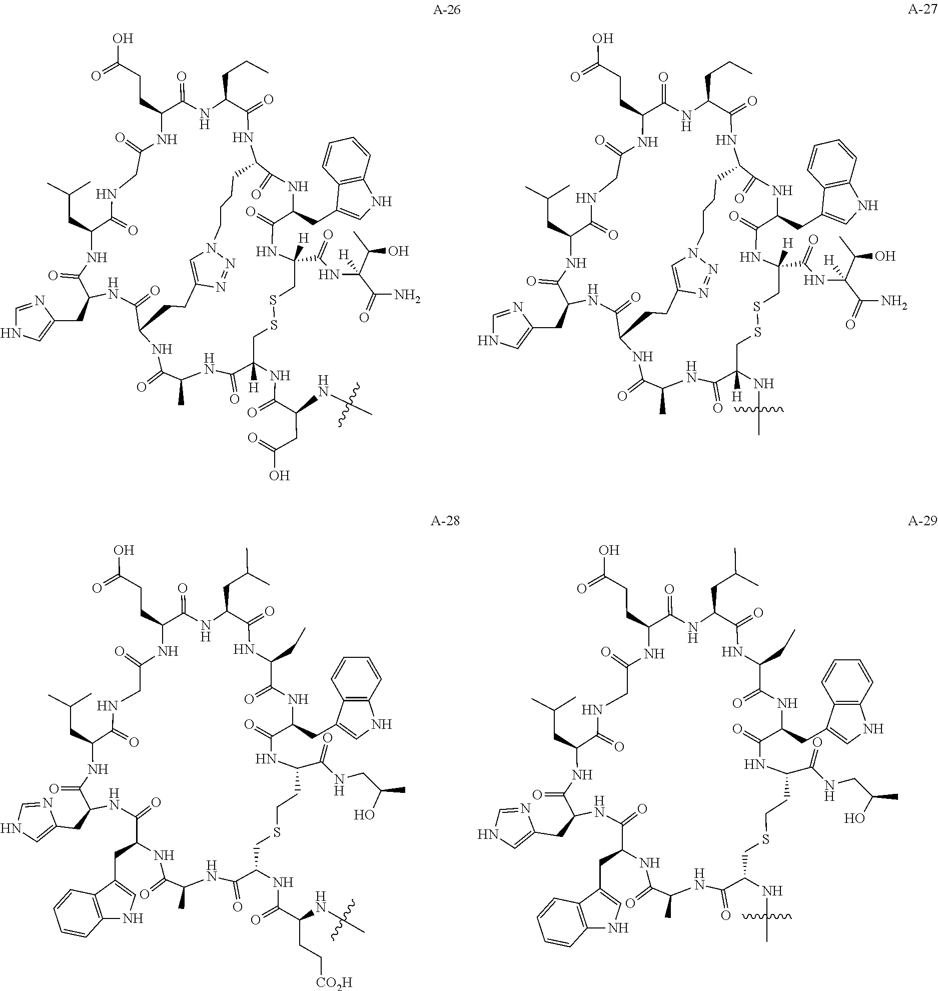

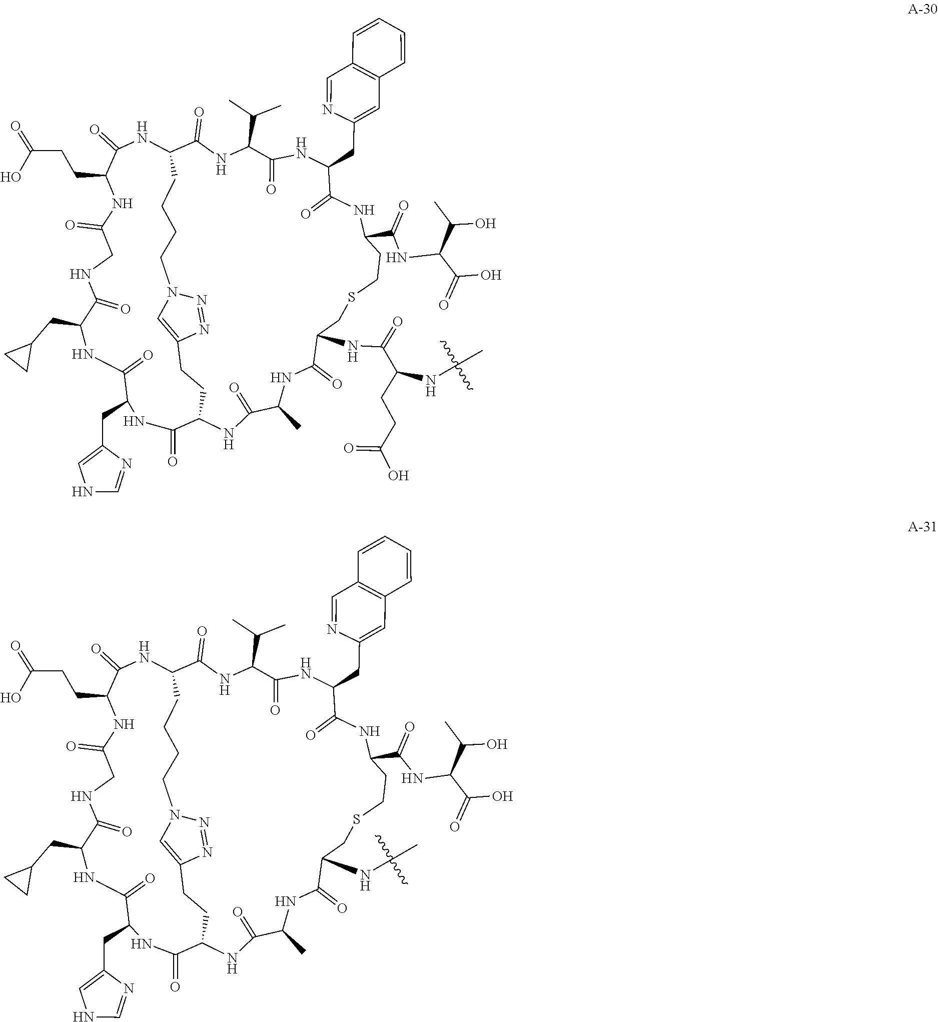 [SMILES: *N[C@@H](CCC(=O)O)C(=O)N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2 ; *N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2]

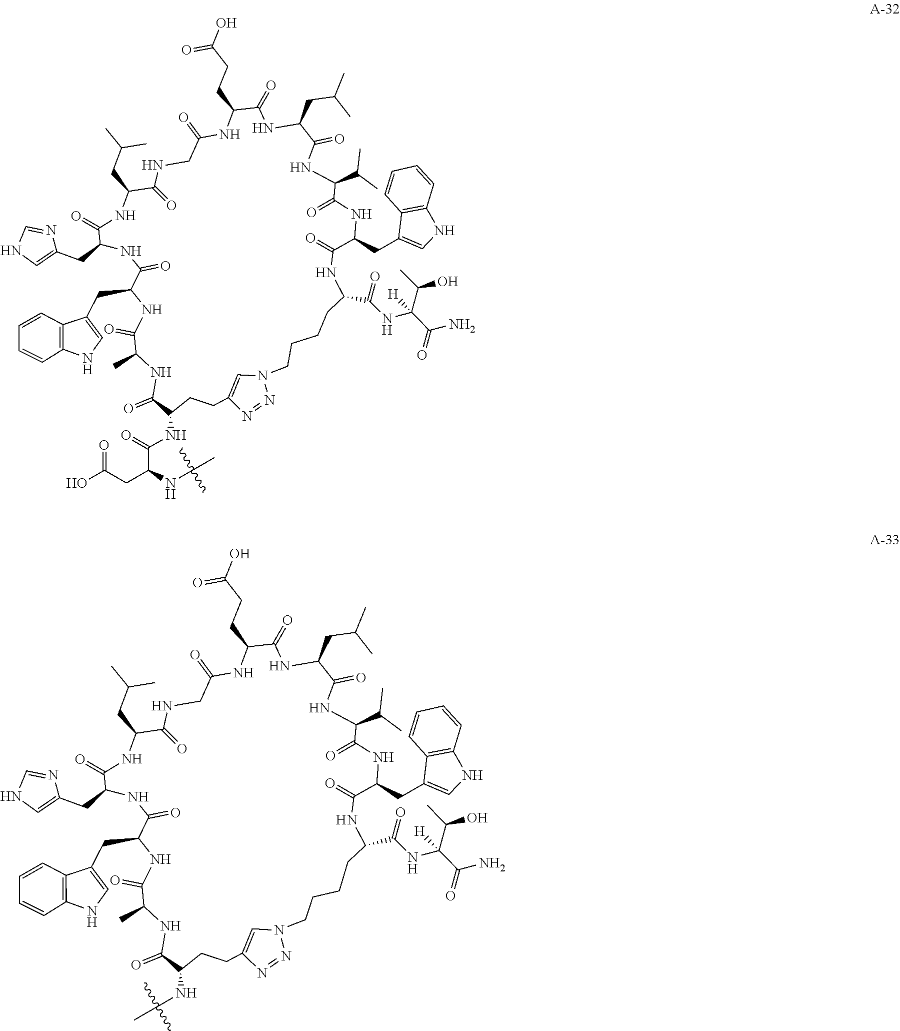

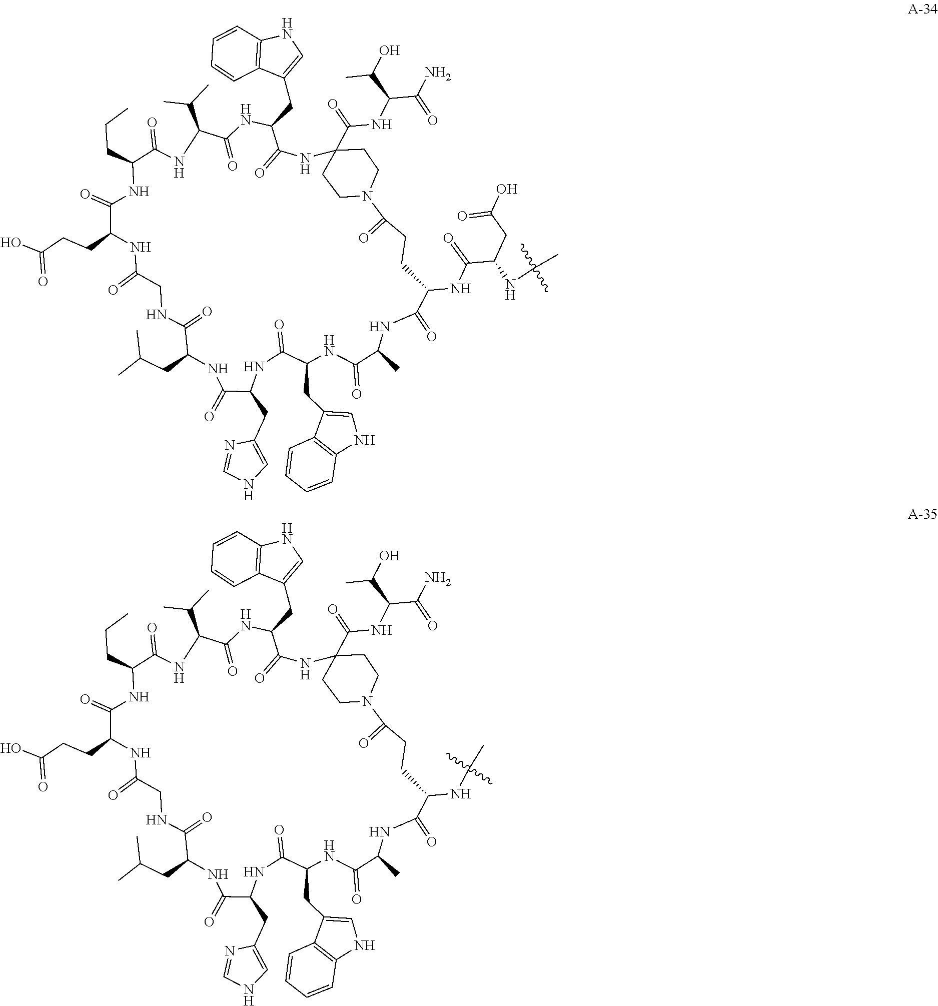 [SMILES: *N[C@@H](CC(=O)O)C(=O)N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O ; *N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O]

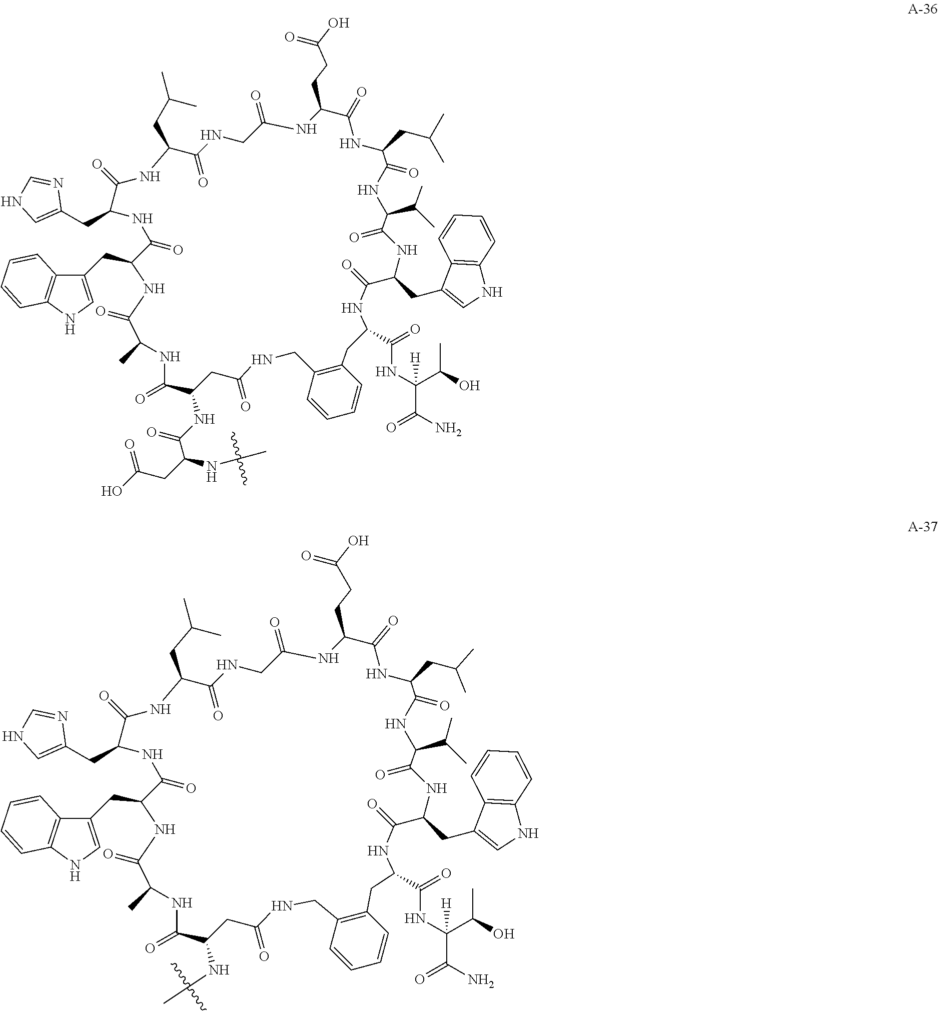

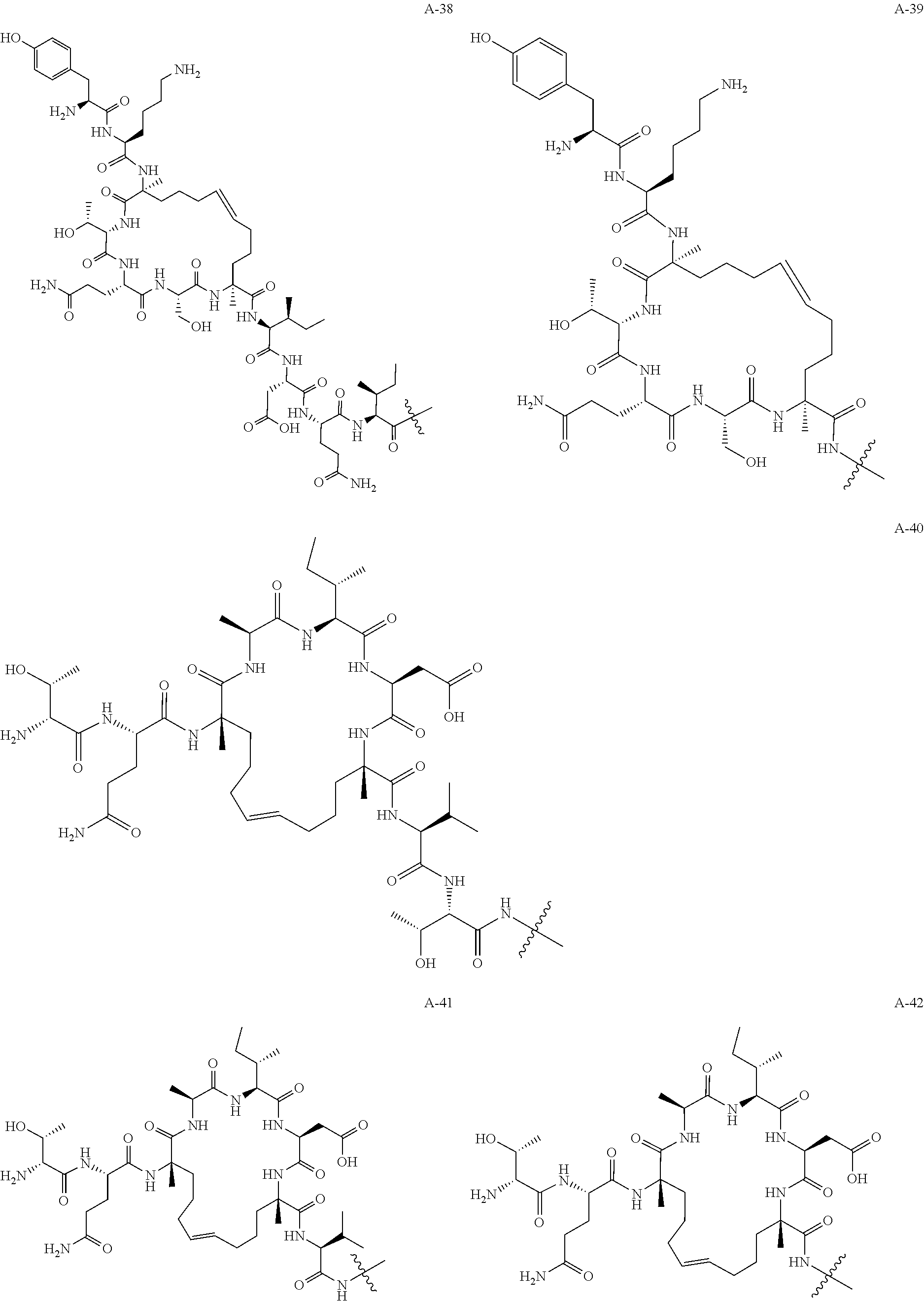

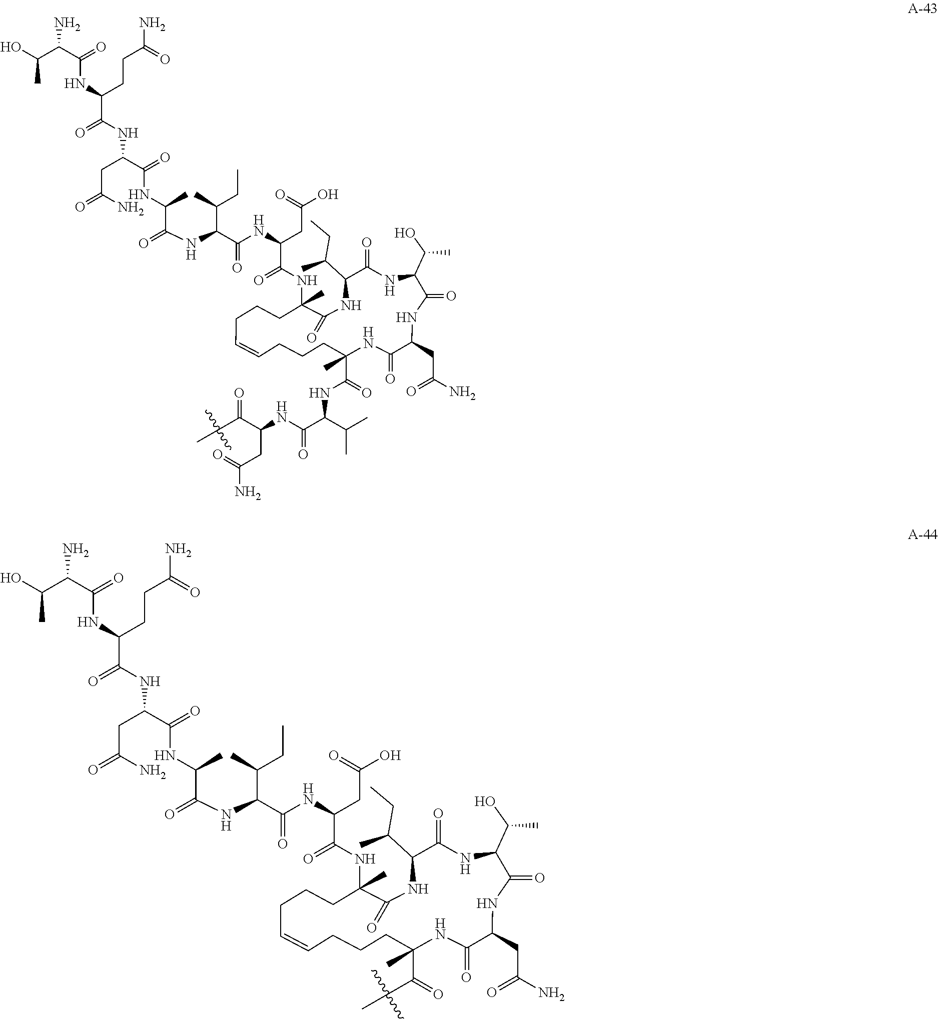

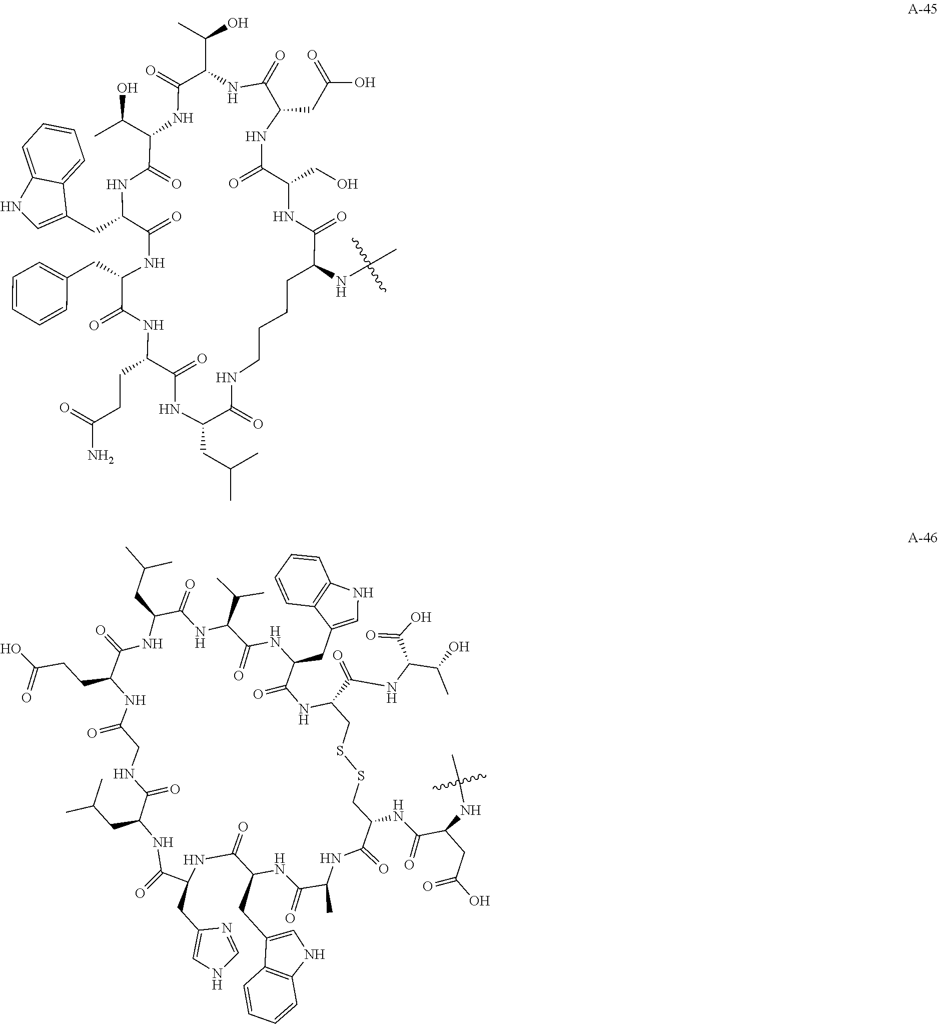 [SMILES: *N[C@H]1CCCCNC(=O)[C@H](CC(C)C)NC(=O)[C@H](CCC(N)=O)NC(=O)[C@H](Cc2ccccc2)NC(=O)[C@H](Cc2c[nH]c3ccccc23)NC(=O)[C@H]([C@@H](C)O)NC(=O)[C@H]([C@@H](C)O)NC(=O)[C@H](CC(=O)O)NC(=O)[C@H](CO)NC1=O ; *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O]

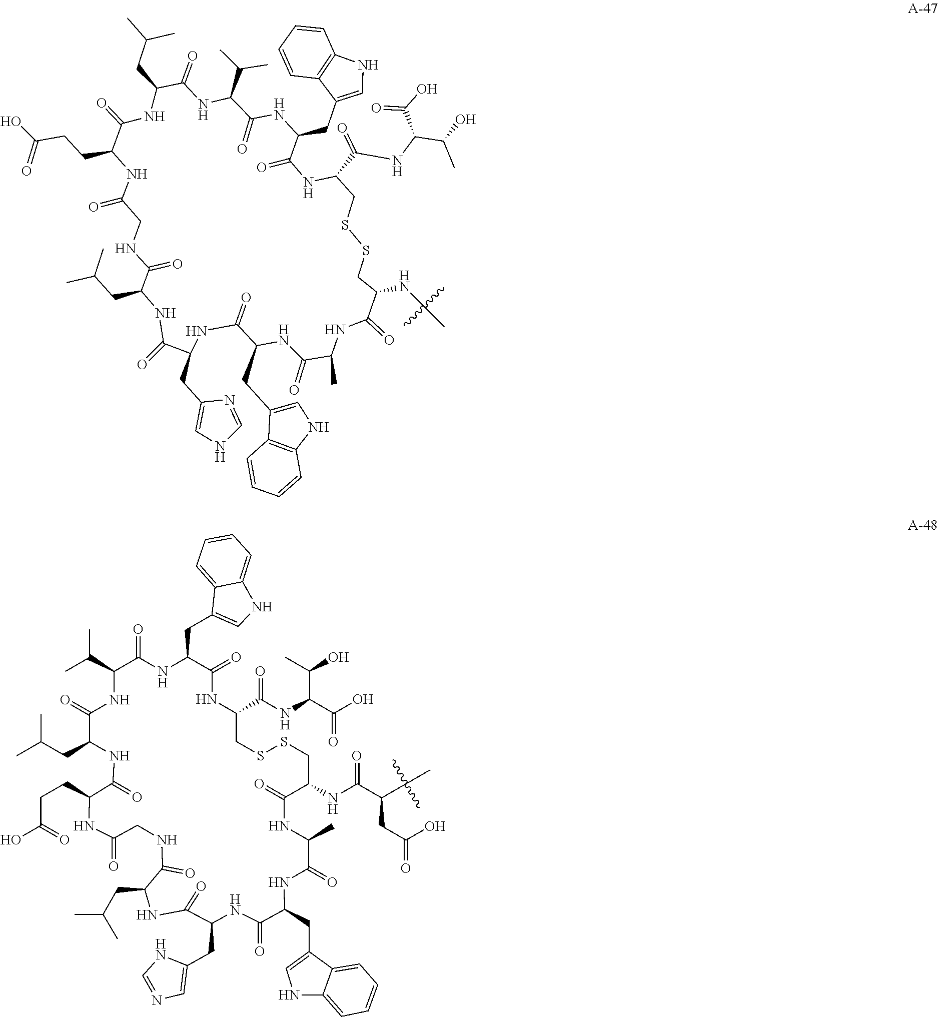 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O ; *[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O]

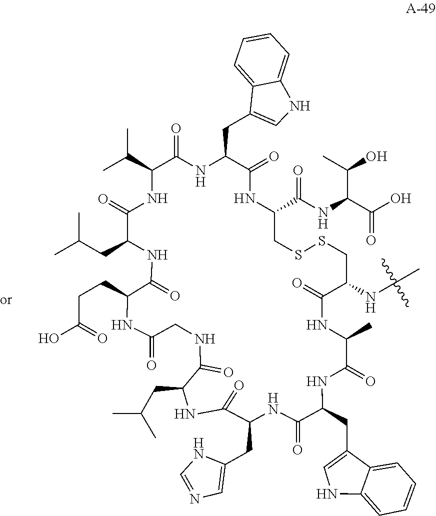 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O]

In some embodiments, a universal antibody binding moiety is or comprises optionally substituted A-1. In some embodiments, a universal antibody binding moiety is or comprises optionally substituted A-2. In some embodiments, a universal antibody binding moiety is or comprises optionally substituted A-3. In some embodiments, a universal antibody binding moiety is or comprises optionally substituted A-4. In some embodiments, a universal antibody binding moiety is or comprises optionally substituted A-5. In some embodiments, a universal antibody binding moiety is or comprises optionally substituted A-6. In some embodiments, a universal antibody binding moiety is or comprises optionally substituted A-7. In some embodiments, a universal antibody binding moiety is or comprises optionally substituted A-8. In some embodiments, a universal antibody binding moiety is or comprises optionally substituted A-9. In some embodiments, a universal antibody binding moiety is or comprises optionally substituted A-10. In some embodiments, a universal antibody binding moiety is or comprises optionally substituted A-11. In some embodiments, a universal antibody binding moiety is or comprises optionally substituted A-12. In some embodiments, a universal antibody binding moiety is or comprises optionally substituted A-13. In some embodiments, a universal antibody binding moiety is or comprises optionally substituted A-14. In some embodiments, a universal antibody binding moiety is or comprises optionally substituted A-15. In some embodiments, a universal antibody binding moiety is or comprises optionally substituted A-16. In some embodiments, a universal antibody binding moiety is or comprises optionally substituted A-17. In some embodiments, a universal antibody binding moiety is or comprises optionally substituted A-18. In some embodiments, a universal antibody binding moiety is or comprises optionally substituted A-19. In some embodiments, a universal antibody binding moiety is or comprises optionally substituted A-20. In some embodiments, a universal antibody binding moiety is or comprises optionally substituted A-21. In some embodiments, a universal antibody binding moiety is or comprises optionally substituted A-22. In some embodiments, a universal antibody binding moiety is or comprises optionally substituted A-23. In some embodiments, a universal antibody binding moiety is or comprises optionally substituted A-24. In some embodiments, a universal antibody binding moiety is or comprises optionally substituted A-25. In some embodiments, a universal antibody binding moiety is or comprises optionally substituted A-26. In some embodiments, a universal antibody binding moiety is or comprises optionally substituted A-27. In some embodiments, a universal antibody binding moiety is or comprises optionally substituted A-28. In some embodiments, a universal antibody binding moiety is or comprises optionally substituted A-29. In some embodiments, a universal antibody binding moiety is or comprises optionally substituted A-30. In some embodiments, a universal antibody binding moiety is or comprises optionally substituted A-31. In some embodiments, a universal antibody binding moiety is or comprises optionally substituted A-32. In some embodiments, a universal antibody binding moiety is or comprises optionally substituted A-33. In some embodiments, a universal antibody binding moiety is or comprises optionally substituted A-34. In some embodiments, a universal antibody binding moiety is or comprises optionally substituted A-35. In some embodiments, a universal antibody binding moiety is or comprises optionally substituted A-36. In some embodiments, a universal antibody binding moiety is or comprises optionally substituted A-37. In some embodiments, a universal antibody binding moiety is or comprises optionally substituted A-38. In some embodiments, a universal antibody binding moiety is or comprises optionally substituted A-39. In some embodiments, a universal antibody binding moiety is or comprises optionally substituted A-40. In some embodiments, a universal antibody binding moiety is or comprises optionally substituted A-41. In some embodiments, a universal antibody binding moiety is or comprises optionally substituted A-42. In some embodiments, a universal antibody binding moiety is or comprises optionally substituted A-43. In some embodiments, a universal antibody binding moiety is or comprises optionally substituted A-44. In some embodiments, a universal antibody binding moiety is or comprises optionally substituted A-45. In some embodiments, a universal antibody binding moiety is or comprises optionally substituted A-46. In some embodiments, a universal antibody binding moiety is or comprises optionally substituted A-47. In some embodiments, a universal antibody binding moiety is or comprises optionally substituted A-48. In some embodiments, a universal antibody binding moiety is or comprises optionally substituted A-49. In some embodiments, it is unsubstituted. In some embodiments, it is substituted.

In some embodiments, a universal antibody binding moiety comprises a peptide unit, and is connected to a linker moiety through the C-terminus of the peptide unit. In some embodiments, it is connected to a linker moiety through the N-terminus of the peptide unit. In some embodiments, it is connected through a side chain group of the peptide unit.

In some embodiments, an antibody binding moiety, e.g., a universal antibody binding moiety, is or comprises a small molecule entity, with a molecular weight of, e.g., less than 10000, 9000, 8000, 7000, 6000, 5000, 4000, 3000, 2000, 1500, 1000, etc. Suitable such antibody binding moieties include small molecule Fc binder moieties, e.g., those described in U.S. Pat. No. 9,745,339, US20130131321, etc.

As appreciated by those skilled in the art, antibodies of various properties and activities (e.g., antibodies recognizing different antigens, having optional modifications, etc.) may be recruited by antibody binding moieties described in the present disclosure. In some embodiments, such antibodies include antibodies administered to a subject, e.g., for therapeutic purposes. In some embodiments, antibodies recruited by antibody binding moieties comprise antibodies toward different antigens. In some embodiments, antibodies recruited by antibody binding moieties comprise antibodies whose antigens are not present on the surface or cell membrane of target cells (e.g., target cells such as cancer cells). In some embodiments, antibodies recruited by antibody binding moieties comprise antibodies which are not targeting antigens present on surface or cell membrane of targets (e.g., target cells such as cancer cells). In some embodiments, antigens on surface of target cells may interfere with the structure, conformation, and/or one or more properties and/or activities of recruited antibodies which bind such antigens. In some embodiments, as appreciated by those skilled in the art, provided technologies comprise universal antibody binding moieties which recruit antibodies of diverse specificities, and no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of recruited antibodies are toward the same antigen, protein, lipid, carbohydrate, etc. Among other things, one advantage of the present disclosure is that provided technologies comprising universal antibody binding moieties can utilize diverse pools of antibodies such as those present in serum. In some embodiments, universal antibody binding moieties of the present disclosure (e.g., those in ARMs) are contacted with a plurality of antibodies, wherein no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of the plurality of antibodies are toward the same antigen, protein, lipid, carbohydrate, etc.

### Amino Acids

In some embodiments, provided compounds and agents may comprise one or more amino acid moieties, e.g., in universal antibody binding moieties, linker moieties, etc. Amino acid moieties can either be those of natural amino acids or unnatural amino acids. In some embodiments, an amino acid has the structure of formula A-I:

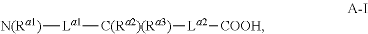

or a salt thereof, wherein each variable is independent as described in the present disclosure. In some embodiments, an amino acid residue has the structure of —N(R^{a1})-L^{a1}-C(R^{a2})(R^{a3})-L^{a2}-CO—.

In some embodiments, Lal is a covalent bond. In some embodiments, a compound of formula A-I is of the structure NH(R^{a1})—C(R^{a2})(R^{a3})-L^{a2}-COOH).

In some embodiments, L^{a2} is a covalent bond. In some embodiments, a compound of formula A-I is of the structure NH(R^{a1})—C(R^{a2})(R^{a3})-L^{a2}-COOH).

In some embodiments, Lal is a covalent bond and L^{a2} is a covalent bond. In some embodiments, a compound of formula A-I is of the structure NH(R^{a1})—C(R^{a2})(R^{a3})—COOH.

In some embodiments, L^{a} is a covalent bond. In some embodiments, L^{a} is optionally substituted C₁₋₆ bivalent aliphatic. In some embodiments, L^{a} is optionally substituted C₁₋₆ alkylene. In some embodiments, L^{a} is —CH₂—. In some embodiments, L^{a} is —CH₂CH₂—. In some embodiments, L^{a} is —CH₂CH₂CH₂—.

In some embodiments, R′ is R. In some embodiments, R^{a1} is R, wherein R is as described in the present disclosure. In some embodiments, R^{a2} is R, wherein R is as described in the present disclosure. In some embodiments, R^{a3} is R, wherein R is as described in the present disclosure. In some embodiments, each of R^{a1}, R^{a2}, and R^{a3} is independently R, wherein R is as described in the present disclosure.

In some embodiments, R^{a1} is hydrogen. In some embodiments, R^{a2} is hydrogen. In some embodiments, R^{a3} is hydrogen. In some embodiments, R^{a1} is hydrogen, and at least one of R^{a2} and R^{a3} is hydrogen. In some embodiments, R^{a1} is hydrogen, one of R^{a2} and R^{a3} is hydrogen, and the other is not hydrogen.

In some embodiments, R^{a2} is -L^{a}-R, wherein R is as described in the present disclosure. In some embodiments, R^{a2} is -L^{a}-R, wherein R is an optionally substituted group selected from C₃₋₃₀ cycloaliphatic, C5-30 aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a2} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a2} is a side chain of an amino acid. In some embodiments, R^{a2} is a side chain of a standard amino acid.

In some embodiments, R^{a3} is -L^{a}-R, wherein R is as described in the present disclosure. In some embodiments, R^{a3} is -L^{a}-R, wherein R is an optionally substituted group selected from C₃₋₃₀ cycloaliphatic, C5-30 aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a3} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a5} is a side chain of an amino acid. In some embodiments, R^{a3} is a side chain of a standard amino acid.

In some embodiments, R is a cyclic group. In some embodiments, R is an optionally substituted C₃₋₃₀ cycloaliphatic group. In some embodiments, R is cyclopropyl.

In some embodiments, R is an aromatic group, and an amino acid residue of an amino acid of formula A-I is a Xaa^{A}. In some embodiments, R is optionally substituted phenyl. In some embodiments, R is phenyl. In some embodiments, R is optionally substituted phenyl. In some embodiments, R is 4-trifluoromethylphenyl. In some embodiments, R is 4-phenylphenyl. In some embodiments, R is optionally substituted 5-30 membered heteroaryl having 1-10 heteroatoms independently selected from oxygen, nitrogen, sulfur, phosphorus and silicon. In some embodiments, R is optionally substituted 5-14 membered heteroaryl having 1-5 heteroatoms independently selected from oxygen, nitrogen, and sulfur. In some embodiments, R is

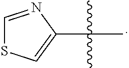 [SMILES: *c1cscn1]

In some embodiments, R is optionally substituted pyridinyl. In some embodiments, R is 1-pyridinyl. In some embodiments, R is 2-pyridinyl. In some embodiments, R is 3-pyridinyl. In some embodiments, R is

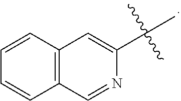 [SMILES: *c1cc2ccccc2cn1]

In some embodiments, R′ is —COOH. In some embodiments, a compound of and an amino acid residue of an amino acid of formula A-I is a Xaa^{N}.

In some embodiments, R′ is —NH₂. In some embodiments, a compound of an amino acid residue of an amino acid of formula A-I is a Xaa^{P}.

In some embodiments, R^{a2} or R^{a3} is R, wherein R is C₁₋₂₀ aliphatic as described in the present disclosure. In some embodiments, a compound of an amino acid residue of an amino acid of formula A-I is a Xaa^{H}. In some embodiments, R is —CH₃. In some embodiments, R is ethyl. In some embodiments, R is propyl. In some embodiments, R is cyclopropyl.

In some embodiments, two or more of R^{a1}, R^{a2}, and R^{a3} are R and are taken together to form an optionally substituted ring as described in the present disclosure.

In some embodiments, R^{a1} and one of R^{a2} and R^{a3} are R and are taken together to form an optionally substituted 3-6 membered ring having no additional ring heteroatom other than the nitrogen atom to which R^{a1} is bonded to. In some embodiments, a formed ring is a 5-membered ring as in proline.

In some embodiments, R^{a2} and R^{a3} are R and are taken together to form an optionally substituted 3-6 membered ring as described in the present disclosure. In some embodiments, R^{a2} and R^{a3} are R and are taken together to form an optionally substituted 3-6 membered ring having one or more nitrogen ring atom. In some embodiments, R^{a2} and R^{a3} are R and are taken together to form an optionally substituted 3-6 membered ring having one and no more than one ring heteroatom which is a nitrogen atom. In some embodiments, a ring is a saturated ring.

In some embodiments, an amino acid is a natural amino acid. In some embodiments, an amino acid is an unnatural amino acid. In some embodiments, an amino acid is an alpha-amino acid. In some embodiments, an amino acid is a beta-amino acid.

### Target

In some embodiments, the present disclosure provides technologies for selectively directing agents comprising target binding moieties (e.g. ARM compounds), antibodies, and immune cells, e.g., NK cells, to desired target sites comprising one or more targets. As those skilled in the art will appreciate, provided technologies are useful for various types of targets.

In some embodiments, targets are damaged or defective tissues. In some embodiments, a target is a damaged tissue. In some embodiments, a target is a defective tissue. In some embodiments, a target is associated with a disease, disorder or condition, e.g., cancer, wound, etc. In some embodiments, a target is a tumor. In some embodiments, targets are or comprise diseased cells. In some embodiments, targets are or comprise cancer cells. In some embodiments, a target is a foreign object. In some embodiments, a target is or comprises an infectious agent. In some embodiments, a target is a microbe. In some embodiments, a target is or comprises bacteria. In some embodiments, a target is or comprises viruses.

In many embodiments, targets are tissues and/or cells associated with diseases, disorders or conditions, particularly various types of cancers. In some embodiments, targets are or comprise cancer cells. Among other things, the present disclosure provides technologies that are particularly useful for selectively targeting cancer cells by the immune system through, e.g., recruitment antibodies (e.g., endogenous antibodies) and immune cells by using ARMs.

Target sites typically comprise one or more physical, chemical and/or biological markers that can be utilized e.g., by target binding moieties of provided compounds (e.g., ARMs), for selectively recruiting antibodies and/or fragments thereof, and/or immune cells to targets.

In some embodiments, cells of target sites comprise one or more characteristic agents that are useful for targeting. In some embodiments, such agents are proteins and/or fragments thereof. In some embodiments, such agents are antigens that are specifically associated with diseases, disorders or conditions.

For example, in some embodiments, cancer cells may comprise one or more tumor-specific antigens or tumor-associated antigens. Target binding moieties as described in the present disclosure can selectively bind to such markers. In some embodiments, target binding moieties of the present disclosure are small molecules which are useful for binding to cell surface proteins and/or proteins within cells.

In some embodiments, characteristic agents, e.g., of cells of the target sites, etc., are or comprise carbohydrates, e.g., those on cell surface, in glycosylated proteins, etc. In some embodiments, characteristic agents are or comprise lipids.

In some embodiments, characteristic agents e.g., of cells of the target sites, etc., are extracellular. In some embodiments, characteristic agents are extracellular proteins. In some embodiments, characteristic agents are on cell surface. In some embodiments, characteristic agents are proteins present on cell surface. For example, in many tumor tissues, cell-surface and/or extracellular mucins show different levels and/or patterns of glycosylation, and may be utilized for targeting.

In some embodiments, targeting sites, e.g., disease tissues, etc., have one or more physical, biological and/or chemical properties that can be utilized by target binding moieties. In some embodiments, such a property is pH. In some embodiments, such a property is concentration of one or more chemical substances. For example, tumor microenvironment is often hypoxic, and/or acidic (e.g., pH 6.5-6.9 v. 7.2-7.4).

In some embodiments, targets are or comprise peptides or fragments thereof. In some embodiments, targets are or comprise proteins or fragments thereof. In some embodiments, a target is avidin. In some embodiments, a target is streptavidin. In some embodiments, a target is or comprises an antigen. In some embodiments, a target is or comprises a tumor-specific antigen. In some embodiments, a target is or comprises a tumor-associated antigen.

In some embodiments, targets are or comprise nucleic acids.

In some embodiments, targets are or comprise lipids.

In some embodiments, targets are or comprise carbohydrates. In some embodiments, targets are or comprise carbohydrates associated with diseases, disorders or conditions. In some embodiments, targets are or comprises carbohydrates associated with cancers, e.g., carbohydrates as glycan modifications of proteins, e.g., on the surface of, or extracellular of, cancer cells.

### Target Binding Moieties

Target binding moieties of various types and chemical classes can be utilized in accordance with the present disclosure, and a number of technologies (e.g., assays, reagents, kits, etc.) for identifying and/or assessing properties of target binding moieties can be utilized in accordance with the present disclosure. Generally, target binding moieties interact with target sites through one or more physical, biological and/or chemical properties. In some embodiments, target binding moieties bind to characteristic agents as described in the present disclosure. In some embodiments, target binding moieties bind to surface, extracellular, and/or intracellular proteins, carbohydrates and/or nucleic acids. In some embodiments, target binding moieties bind to surface proteins of target cells. In some embodiments, target binding moieties are small molecule moieties. In some embodiments, target binding moieties are antibody agents. In some embodiments, target binding moieties are nucleic acid agents such as aptamers. In some embodiments, target binding moieties are lipid moieties. Certain types of target binding moieties are described below; those skilled in the art appreciates that other types of target binding moieties, including many known in the art, can also be utilized in accordance with the present disclosure.

In some embodiments, targeting binding moieties bind to targets through one or more proteins, lipids, nucleic acids, carbohydrates, small molecules, etc. of the targets. For example, in some embodiments, target binding moieties bind to tumor-specific antigens of target cancer cells. In some embodiments, a tumor-specific antigen is or comprises carbohydrate or a fragment thereof. In some embodiments, a tumor-specific antigen is or comprises a protein or a fragment thereof.

a. Small Molecules

In some embodiments, a target binding moiety is a small molecule moiety. In some embodiments, a small molecule moiety has a molecular weight no more than 8000, 7000, 6000, 5000, 4000, 3000, 2000, 1500, 1000, 900, 800, 700, or 600. In some embodiments, a small molecule moiety has a molecular weight no more than 8000. In some embodiments, a small molecule moiety has a molecular weight no more than 7000. In some embodiments, a small molecule moiety has a molecular weight no more than 6000. In some embodiments, a small molecule moiety has a molecular weight no more than 5000. In some embodiments, a small molecule moiety has a molecular weight no more than 4000. In some embodiments, a small molecule moiety has a molecular weight no more than 3000. In some embodiments, a small molecule moiety has a molecular weight no more than 2000. In some embodiments, a small molecule moiety has a molecular weight no more than 1500. In some embodiments, a small molecule moiety has a molecular weight no more than 1000. In some embodiments, a small molecule moiety has a molecular weight no more than 900. Among other things, the present disclosure encompasses the recognition that small molecule target binding moieties may be able to bind to markers outside of, on the surface of, and/or inside of targets, e.g., cancer cells.

In some embodiments, a small molecule target binding moiety is or comprises a moiety that selectively binds to a protein or a fragment thereof, e.g., cancer antigen. For example, in some embodiments, a target binding moiety is or comprises a moiety that selectively binds to prostate-specific membrane antigen (PSMA). In some embodiments, a target binding moiety is or comprises

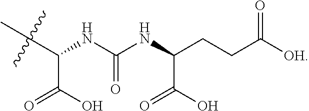 [SMILES: *[C@H](NC(=O)N[C@@H](CCC(=O)O)C(=O)O)C(=O)O]

In some embodiments, a small molecule target binding moiety is or comprises a biotin moiety. In some embodiments, a small molecule target binding moiety is or comprises

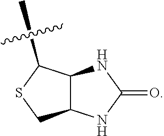

In some embodiments, a small molecule target binding moiety is or comprises

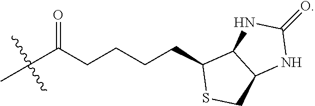 [SMILES: *C(=O)CCCC[C@@H]1SC[C@@H]2NC(=O)N[C@@H]21]

b. Peptide Agents

In some embodiments, a target binding moiety is or comprises a peptide agent. In some embodiments, a target binding moiety is a peptide moiety. In some embodiments, a peptide moiety can either be linier or cyclic. In some embodiments, a target binding moiety is or comprises a cyclic peptide moiety. Various peptide target binding moieties are known in the art and can be utilized in accordance with the present disclosure.

In some embodiments, a target binding moiety is or comprises a peptide aptamer agent.

c. Aptamer Agents

In some embodiments, a target binding moiety is or comprises a nucleic acid agent. In some embodiments, a target binding moiety is or comprises an oligonucleotide moiety. In some embodiments, a target binding moiety is or comprises an aptamer agent. Various aptamer agents are known in the art or can be readily developed using common technologies, and can be utilized in provided technologies in accordance with the present disclosure.

### Linker Moieties

In some embodiments, antibody binding moieties are optionally connected to target binding moieties through linker moieties. Linker moieties of various types and/or for various purposes, e.g., those utilized in antibody-drug conjugates, etc., may be utilized in accordance with the present disclosure.

Linker moieties can be either bivalent or polyvalent. In some embodiments, a linker moiety is bivalent. In some embodiments, a linker is polyvalent and connecting more than two moieties.

In some embodiments, a linker moiety is L. In some embodiments, L is a covalent bond, or a bivalent or polyvalent optionally substituted, linear or branched C₁-100 group comprising one or more aliphatic, aryl, heteroaliphatic having 1-20 heteroatoms, heteroaromatic having 1-20 heteroatoms, or any combinations thereof, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O) C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′), —P(S)(R′)—, —P(S)(NR′)—, —P(R′)—, —P(OR′)—, —P(SR′)—, —P(NR′)—, or [(—O—C(R′)₂—C(R′)₂—)ₙ]—, wherein n is 1-20.

In some embodiments, L is bivalent. In some embodiments, L is a bivalent or optionally substituted, linear or branched group selected from C₁-C₀₀ aliphatic and C₁₋₁₀₀ heteroaliphatic having 1-50 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O) C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′), —P(S)(OR′)—, —P(S)(SR′)—, —P(S)(R′)—, —P(S)(NR′)—, —P(R′)—, —P(OR′)—, —P(SR′)—, —P(NR′)—, or -[(—O—C(R′)₂—C(R′)₂—)ₙ]—.

In some embodiments, L is a covalent bond. In some embodiments, L is a bivalent optionally substituted, linear or branched C₁-100 aliphatic group wherein one or more methylene units of the group are optionally and independently replaced. In some embodiments, L is a bivalent optionally substituted, linear or branched C₆₋₁₀₀ arylaliphatic group wherein one or more methylene units of the group are optionally and independently replaced. In some embodiments, L is a bivalent optionally substituted, linear or branched C₅₋₁₀₀ heteroarylaliphatic group having 1-20 hetereoatoms wherein one or more methylene units of the group are optionally and independently replaced. In some embodiments, L is a bivalent optionally substituted, linear or branched C₁-100 heteroaliphatic group having 1-20 heteroatoms wherein one or more methylene units of the group are optionally and independently replaced.

In some embodiments, a linker moiety (e.g., L) is or comprises one or more polyethylene glycol units. In some embodiments, a linker moiety is or comprises —(CH₂CH₂O)ₙ—, wherein n is as described in the present disclosure. In some embodiments, one or more methylene units of L are independently replaced with —(CH₂CH₂O)ₙ—. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, a linker moiety comprises one or more moieties, e.g., amino, carbonyl, etc., that can be utilized for connection with other moieties. In some embodiments, a linker moiety comprises one or more —NR′—, wherein R′ is as described in the present disclosure. In some embodiments, —NR′-improves solubility. In some embodiments, —NR′— serves as connection points to another moiety. In some embodiments, R′ is —H. In some embodiments, one or more methylene units of L are independently replaced with —NR′—, wherein R′ is as described in the present disclosure.

In some embodiments, a linker moiety, e.g., L, comprises a —C(O)— group, which can be utilized for connections with a moiety. In some embodiments, one or more methylene units of L are independently replaced with —C(O)—.

In some embodiments, a linker moiety is or comprises one or more ring moieties, e.g., one or more methylene units of L are replaced with -Cy-. In some embodiments, a linker moiety, e.g., L, comprises an aryl ring. In some embodiments, a linker moiety, e.g., L, comprises an heteroaryl ring. In some embodiments, a linker moiety, e.g., L, comprises an aliphatic ring. In some embodiments, a linker moiety, e.g., L, comprises an heterocyclyl ring. In some embodiments, a linker moiety, e.g., L, comprises a polycyclic ring. In some embodiments, a ring in a linker moiety, e.g., L, is 3-20 membered. In some embodiments, a ring is 5-membered. In some embodiments, a ring is 6-membered. In some embodiments, a ring in a linker is product of a cycloaddition reaction (e.g., click chemistry, and variants thereof) utilized to link different moieties together.

In some embodiments, a linker moiety (e.g., L) is or comprises

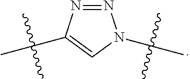 [SMILES: *c1cn(*)nn1]

In some embodiments, a methylene unit of L is replaced with

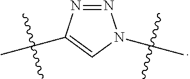 [SMILES: *c1cn(*)nn1]

In some embodiments, -Cy- is

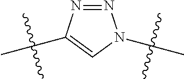 [SMILES: *c1cn(*)nn1]

In some embodiments, a linker moiety is as described in Table 1. Additional linker moiety, for example, include those described for L². In some embodiments, L is L¹ ad present disclosure. In some embodiments, L is L² as described in the present disclosure. In some embodiments, L is L³ as described in the present disclosure. In some embodiments, L is L^{b} as described in the present disclosure.

In some embodiments, L is

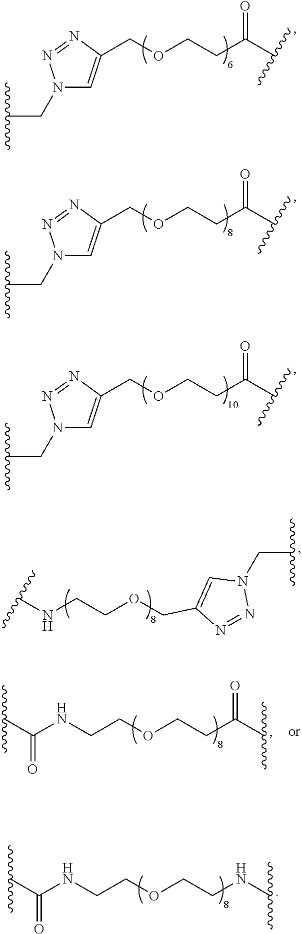

### Certain Embodiments of Variables

As examples, exemplary embodiments of variables are described throughout the present disclosure. As appreciated by those skilled in the art, embodiments for different variables may be optionally combined.

As defined above and described herein, ABT is an antibody binding moiety.

In some embodiments, ABT is an antibody binding moiety.

In some embodiments, ABT is selected from those depicted in Table 1, below.

As defined above and described herein, L is a bivalent linker moiety that connects ABT with TBT.

In some embodiments, L is a bivalent linker moiety that connects ABT with TBT.

In some embodiments, L is selected from those depicted in Table 1, below.

As defined above and described herein, TBT is a target binding moiety.

In some embodiments, TBT is a target binding moiety.

In some embodiments, TBT is selected from those depicted in Table 1, below.

As defined above and described herein, each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or: R¹ and R^{1′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; R³ and R^{3′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; an R⁵ group and the R^{5′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; or two R⁵ groups are optionally taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy-, wherein each -Cy¹- is independently a 5-6 membered heteroarylenyl with 1-4 heteroatoms independently selected from nitrogen, oxygen or sulfur.

In some embodiments, R¹ is hydrogen. In some embodiments, R¹ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, or sulfur. In some embodiments, R¹ is an optionally substituted 5-6 membered monocyclic heteroaromatic ring having 1-4 heteroatoms independently selected from nitrogen, oxygen, or sulfur. In some embodiments, R¹ is an optionally substituted 8-10 membered bicyclic heteroaromatic ring having 1-5 heteroatoms independently selected from nitrogen, oxygen, or sulfur.

In some embodiments, R¹ is

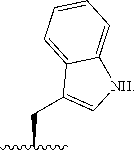

In some embodiments, R¹ is

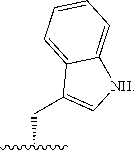

In some embodiments, R¹ is

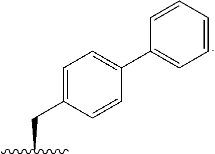

In some embodiments, R¹ is

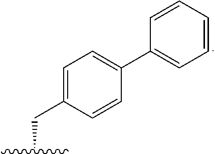

In some embodiments, R¹ is

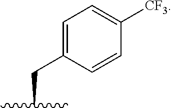

In some embodiments, R¹ is

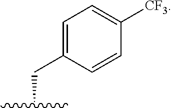

In some embodiments, R¹ is

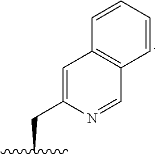

In some embodiments, R¹ is

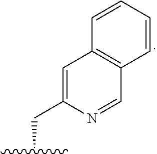

In some embodiments, R¹ is

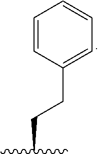

In some embodiments, R¹ is

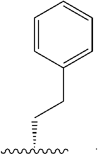

In some embodiments, R¹ is

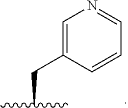

In some embodiments, R¹ is

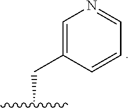

In some embodiments, R¹ is

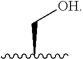

In some embodiments, R¹ is

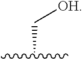

In some embodiments, R¹ is

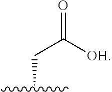

In some embodiments, R¹ is

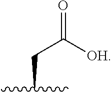

In some embodiments, R¹ is

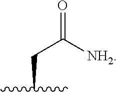

In some embodiments, R¹ is

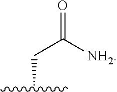

In some embodiments, R¹ is

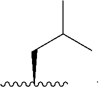

In some embodiments, R¹ is

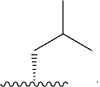

In some embodiments, R¹ and R^{1′} are optionally taken together with their intervening carbon atom to form a 3-8 membered saturated or partially unsaturated spirocyclic carbocyclic ring. In some embodiments, R¹ and R^{1′} are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R¹ is selected from those depicted in Table 1, below.

In some embodiments, R is R¹ as described in the present disclosure. In some embodiments, R^{a2} is R¹ as described in the present disclosure. In some embodiments, R^{a3} is R¹ as described in the present disclosure.

In some embodiments, R³ is hydrogen. In some embodiments, R³ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R³ is an optionally substituted C₁₋₆ aliphatic group. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, or sulfur. In some embodiments, R³ is an optionally substituted 5-6 membered monocyclic heteroaromatic ring having 1-4 heteroatoms independently selected from nitrogen, oxygen, or sulfur. In some embodiments, R³ is an optionally substituted 8-10 membered bicyclic heteroaromatic ring having 1-5 heteroatoms independently selected from nitrogen, oxygen, or sulfur.

In some embodiments, R³ is methyl. In some embodiments, R³ is

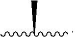

In some embodiments, R³ is

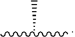

In some embodiments, R³ is

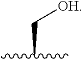

In some embodiments, R³ is

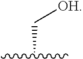

In some embodiments, R³ is

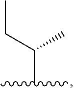 [SMILES: *[C@@H](C)CC]

wherein the site of attachment has(S) stereochemistry. In some embodiments, R³ is

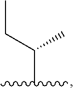 [SMILES: *[C@@H](C)CC]

wherein the site of attachment has (R) stereochemistry. In some embodiments, R³ is

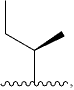 [SMILES: *[C@H](C)CC]

wherein the site of attachment has(S) stereochemistry. In some embodiments, R³ is

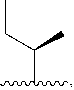 [SMILES: *[C@H](C)CC]

wherein the site of attachment has (R) stereochemistry.

In some embodiments, R³ is

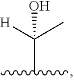 [SMILES: *[C@@H](C)O]

wherein the site of attachment has (S) stereochemistry. In some embodiments, R³ is

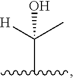 [SMILES: *[C@@H](C)O]

wherein the site of attachment has (R) stereochemistry.

In some embodiments, R³ and R^{3′} are optionally taken together with their intervening carbon atom to form a 3-8 membered saturated or partially unsaturated spirocyclic carbocyclic ring. In some embodiments, R³ and R^{3′} are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R³ is selected from those depicted in Table 1, below.

In some embodiments, R is R² as described in the present disclosure. In some embodiments, R^{a2} is R² as described in the present disclosure. In some embodiments, R^{a3} is R² as described in the present disclosure.

In some embodiments, R⁵ is hydrogen. In some embodiments, R⁵ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R⁵ is an optionally substituted C₁₋₆ aliphatic group. In some embodiments, R⁵ is an optionally substituted 3-8 membered saturated or partially unsaturated monocyclic carbocyclic ring. In some embodiments, R⁵ is an optionally substituted phenyl. In some embodiments, R⁵ is an optionally substituted 8-10 membered bicyclic aromatic carbocyclic ring. In some embodiments, R⁵ is an optionally substituted 4-8 membered saturated or partially unsaturated monocyclic heterocyclic ring having 1-2 heteroatoms independently selected from nitrogen, oxygen, or sulfur. In some embodiments, R⁵ is an optionally substituted 5-6 membered monocyclic heteroaromatic ring having 1-4 heteroatoms independently selected from nitrogen, oxygen, or sulfur. In some embodiments, R⁵ is an optionally substituted 8-10 membered bicyclic heteroaromatic ring having 1-5 heteroatoms independently selected from nitrogen, oxygen, or sulfur.

In some embodiments, R⁵ is methyl. In some embodiments, R⁵ is

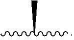

In some embodiments, R⁵ is

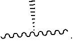

In some embodiments, R⁵ is

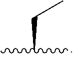

In some embodiments, R⁵ is

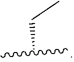

In some embodiments, R⁵ is

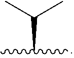

In some embodiments, R⁵ is

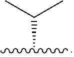

In some embodiments, R⁵ is

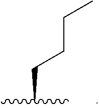

In some embodiments, R⁵ is

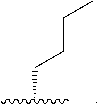

In some embodiments, R⁵ is

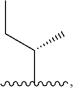 [SMILES: *[C@@H](C)CC]

wherein the site of attachment has(S) stereochemistry. In some embodiments, R⁵ is

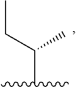 [SMILES: *[C@@H](C)CC]

wherein the site of attachment has (R) stereochemistry. In some embodiments, R⁵ is

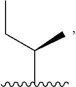 [SMILES: *[C@H](C)CC]

wherein the site of attachment has(S) stereochemistry. In some embodiments, R⁵ is

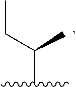 [SMILES: *[C@H](C)CC]

wherein the site of attachment has (R) stereochemistry. In some embodiments, R⁵ is

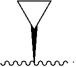

In some embodiments, R⁵ is

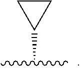

In some embodiments, R⁵ is

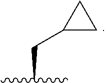

In some embodiments, R⁵ is

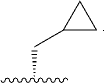

In some embodiments, R⁵ is

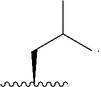

In some embodiments, R⁵ is

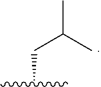

In some embodiments, R⁵ is

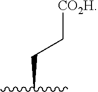

In some embodiments, R⁵ is

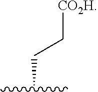

In some embodiments, R⁵ is

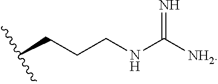

In some embodiments, R⁵ is

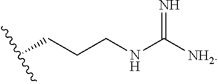

In some embodiments, R⁵ is

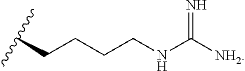

In some embodiments, R⁵ is

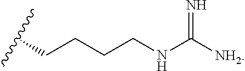

In some embodiments, R⁵ is

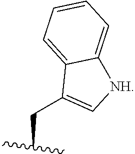

In some embodiments, R⁵ is

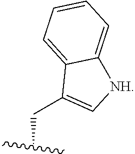

In some embodiments, R⁵ is

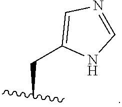

In some embodiments, R⁵ is

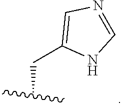

In some embodiments, R⁵ is

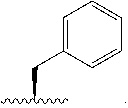

In some embodiments, R⁵ is

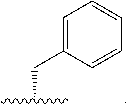

In some embodiments, R⁵ is

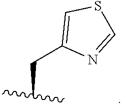

In some embodiments, R⁵ is

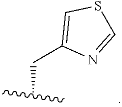

In some embodiments, R⁵ is

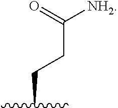

In some embodiments, R⁴ is 5

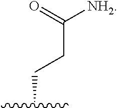

In some embodiments, R⁵ is

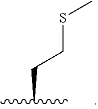

In some embodiments, R⁵ is

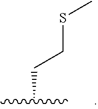

In some embodiments, R⁵ is

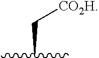

In some embodiments, R⁵ is

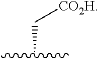

In some embodiments, R⁴ is

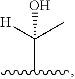 [SMILES: *[C@@H](C)O]

wherein the site of attachment has(S) stereochemistry. In some embodiments, R⁴ is

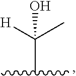 [SMILES: *[C@@H](C)O]

wherein the site of attachment has (R) stereochemistry.

In some embodiments, R⁵ and the R^{5′} group attached to the same carbon atom are optionally taken together with their intervening carbon atom to form a 3-8 membered saturated or partially unsaturated spirocyclic carbocyclic ring. In some embodiments, R⁵ and the R^{5′} group attached to the same carbon atom are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, two R⁵ groups are taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy- is independently a 5-6 membered heteroarylenyl with 1-4 heteroatoms independently selected from nitrogen, oxygen or sulfur.

In some embodiments, two R′ groups are taken together with their intervening atoms to form

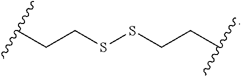 [SMILES: *CCSSCC*]

In some embodiments, two R⁵ groups are taken together with their intervening atoms to form

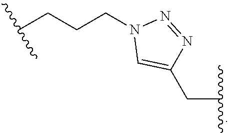 [SMILES: *CCCn1cc(C*)nn1]

In some embodiments, two R³ groups are taken together with their intervening atoms to form

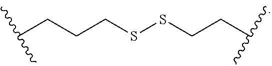 [SMILES: *CCCSSCC*]

In some embodiments, two R⁵ groups are taken together with their intervening atoms to form

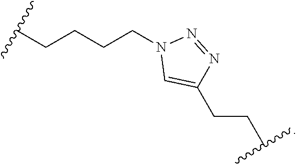 [SMILES: *CCCCn1cc(CC*)nn1]

In some embodiments, R⁵ is selected from those depicted in Table 1, below.

In some embodiments, R is R⁵ as described in the present disclosure. In some embodiments, R^{a2} is R⁵ as described in the present disclosure. In some embodiments, R^{a3} is R⁵ as described in the present disclosure.

As defined above and described herein, each of R^{1′}, R^{3′} and R^{5′} is independently hydrogen or C₁₋₃ aliphatic.

In some embodiments, R^{1′} is hydrogen. In some embodiments, R^{1′} is C₁₋₃ aliphatic.

In some embodiments, R^{1′} is methyl. In some embodiments, R^{1′} is ethyl. In some embodiments, R^{1′} is n-propyl. In some embodiments, R^{1′} is isopropyl. In some embodiments, R^{1′} is cyclopropyl.

In some embodiments, R^{1′} is selected from those depicted in Table 1, below.

In some embodiments, R^{3′} is hydrogen. In some embodiments, R^{3′} is C₁₋₃ aliphatic.

In some embodiments, R^{3′} is methyl. In some embodiments, R^{3′} is ethyl. In some embodiments, R^{3′} is n-propyl. In some embodiments, R^{3′} is isopropyl. In some embodiments, R^{3′ ′} is cyclopropyl.

In some embodiments, R^{3′} is selected from those depicted in Table 1, below.

In some embodiments, R^{5′} is hydrogen. In some embodiments, R^{5′} is C₁₋₃ aliphatic.

In some embodiments, R^{5′} is methyl. In some embodiments, R^{5′} is ethyl. In some embodiments, R^{5′ ′} is n-propyl. In some embodiments, R^{5′ ′} is isopropyl. In some embodiments, R^{5′ ′} is cyclopropyl.

In some embodiments, R^{5′} is selected from those depicted in Table 1, below.

As defined above and described herein, each of R², R⁴ and R⁶ is independently hydrogen, or C₁₋₄ aliphatic, or: R² and R¹ are optionally taken together with their intervening atoms to form a 4-8 membered saturated or partially unsaturated monocyclic heterocyclic ring having 1-2 heteroatoms independently selected from nitrogen, oxygen, or sulfur; R⁴ and R³ are optionally taken together with their intervening atoms to form a 4-8 membered saturated or partially unsaturated monocyclic heterocyclic ring having 1-2 heteroatoms independently selected from nitrogen, oxygen, or sulfur; or an R⁶ group and its adjacent R⁵ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R² is hydrogen. In some embodiments, R² is C₁₋₄ aliphatic. In some embodiments, R² is methyl. In some embodiments, R² is ethyl. In some embodiments, R² is n-propyl. In some embodiments, R² is isopropyl. In some embodiments, R² is n-butyl. In some embodiments, R² is isobutyl. In some embodiments, R² is tert-butyl.

In some embodiments, R² and R¹ are taken together with their intervening atoms to form a 4-8 membered saturated or partially unsaturated monocyclic heterocyclic ring having 1-2 heteroatoms independently selected from nitrogen, oxygen, or sulfur.

In some embodiments, R² and R¹ are taken together with their intervening atoms to form

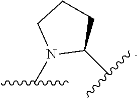 [SMILES: *[C@@H]1CCCN1*]

In some embodiments, R² and R¹ are taken together with their intervening atoms to form

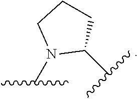 [SMILES: *[C@H]1CCCN1*]

In some embodiments, R² is selected from those depicted in Table 1, below.

In some embodiments, R⁴ is hydrogen. In some embodiments, R⁴ is C₁₋₄ aliphatic. In some embodiments, R⁴ is methyl. In some embodiments, R⁴ is ethyl. In some embodiments, R⁴ is n-propyl. In some embodiments, R⁴ is isopropyl. In some embodiments, R⁴ is n-butyl. In some embodiments, R⁴ is isobutyl. In some embodiments, R⁴ is tert-butyl.

In some embodiments, R⁴ and R³ are taken together with their intervening atoms to form a 4-8 membered saturated or partially unsaturated monocyclic heterocyclic ring having 1-2 heteroatoms independently selected from nitrogen, oxygen, or sulfur.

In some embodiments, R⁴ and R³ are taken together with their intervening atoms to form

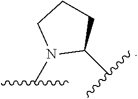 [SMILES: *[C@@H]1CCCN1*]

In some embodiments, R⁴ and R³ are taken together with their intervening atoms to form

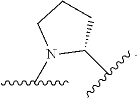 [SMILES: *[C@H]1CCCN1*]

In some embodiments, R″ is selected from those depicted in Table 1, below.

In some embodiments, R⁶ is hydrogen. In some embodiments, R⁶ is C₁₋₄ aliphatic. In some embodiments, R⁶ is methyl. In some embodiments, R⁶ is ethyl. In some embodiments, R⁶ is n-propyl. In some embodiments, R⁶ is isopropyl. In some embodiments, R⁶ is n-butyl. In some embodiments, R⁶ is isobutyl. In some embodiments, R⁶ is tert-butyl.

In some embodiments, an R⁶ group and its adjacent R⁵ group are taken together with their intervening atoms to form a 4-8 membered saturated or partially unsaturated monocyclic heterocyclic ring having 1-2 heteroatoms independently selected from nitrogen, oxygen, or sulfur.

In some embodiments, an R⁶ group and its adjacent R³ group are taken together with their intervening atoms to form

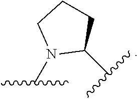 [SMILES: *[C@@H]1CCCN1*]

In some embodiments, an R^{c} group and its adjacent R⁵ group are taken together with their intervening atoms to form

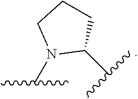 [SMILES: *[C@H]1CCCN1*]

In some embodiments, R^{e} is selected from those depicted in Table 1, below.

In some embodiments, R is R″ as described in the present disclosure. In some embodiments, R^{a2} is R″ as described in the present disclosure. In some embodiments, R^{a3} is R^{1′} as described in the present disclosure. In some embodiments, R is R³ as described in the present disclosure. In some embodiments, R^{a2} is R^{3′} as described in the present disclosure. In some embodiments, R^{a3} is R³ as described in the present disclosure. In some embodiments, R is R² as described in the present disclosure. In some embodiments, R^{a2} is R² as described in the present disclosure. In some embodiments, R^{a3} is R² as described in the present disclosure. In some embodiments, R is R⁴ as described in the present disclosure. In some embodiments, R^{a2} is R⁴ as described in the present disclosure. In some embodiments, R^{a3} is R⁴ as described in the present disclosure. In some embodiments, R is R^{c} as described in the present disclosure. In some embodiments, R^{a2} is R⁶ as described in the present disclosure. In some embodiments, R^{a3} is R⁶ as described in the present disclosure.

As defined above and described herein, L¹ is a trivalent linker moiety that connects

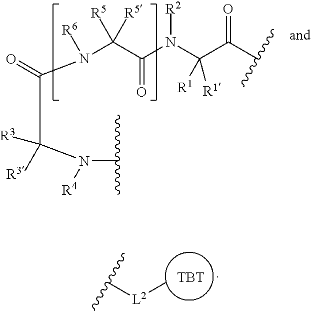

In some embodiments, L¹ is

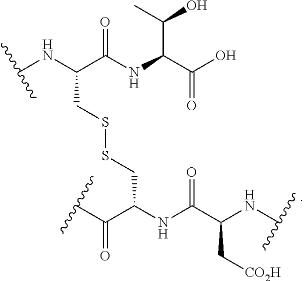 [SMILES: *N[C@@H](CC(=O)O)C(=O)N[C@@H](CSSC[C@H](N*)C(=O)N[C@H](C(=O)O)[C@@H](C)O)C(*)=O]

In some embodiments, L¹ is

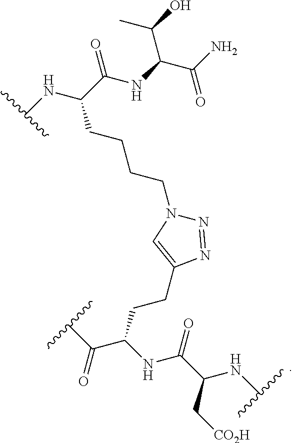 [SMILES: *N[C@@H](CC(=O)O)C(=O)N[C@@H](CCc1cn(CCCC[C@H](N*)C(=O)N[C@H](C(N)=O)[C@@H](C)O)nn1)C(*)=O]

In some embodiments, L¹ is

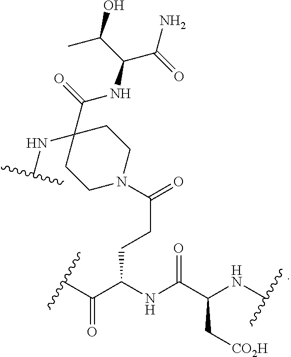 [SMILES: *N[C@@H](CC(=O)O)C(=O)N[C@@H](CCC(=O)N1CCC(N*)(C(=O)N[C@H](C(N)=O)[C@@H](C)O)CC1)C(*)=O]

In some embodiments, L¹ is

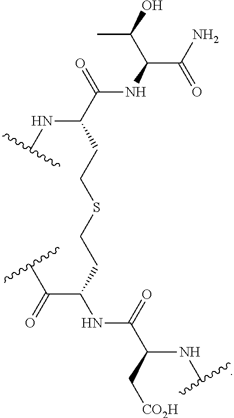 [SMILES: *N[C@@H](CC(=O)O)C(=O)N[C@@H](CCSCC[C@H](N*)C(=O)N[C@H](C(N)=O)[C@@H](C)O)C(*)=O]

In some embodiments, L¹ is

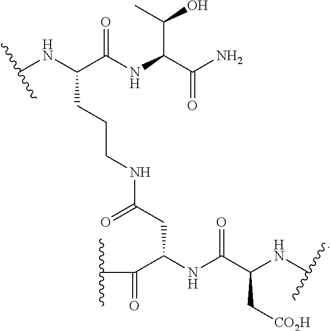 [SMILES: *N[C@@H](CC(=O)O)C(=O)N[C@@H](CC(=O)NCCC[C@H](N*)C(=O)N[C@H](C(N)=O)[C@@H](C)O)C(*)=O]

In some embodiments, L¹ is

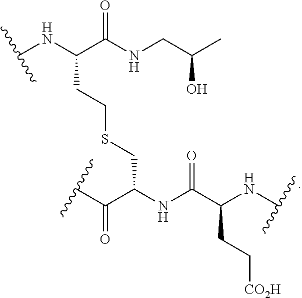 [SMILES: *N[C@@H](CCSC[C@H](NC(=O)[C@H](CCC(=O)O)N*)C(*)=O)C(=O)NC[C@@H](C)O]

In some embodiments, L¹ is

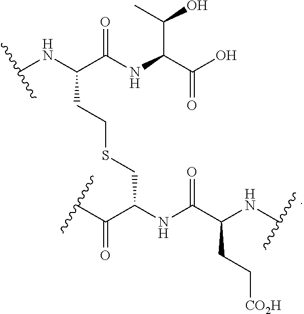 [SMILES: *N[C@@H](CCC(=O)O)C(=O)N[C@@H](CSCC[C@H](N*)C(=O)N[C@H](C(=O)O)[C@@H](C)O)C(*)=O]

In some embodiments, L¹ is

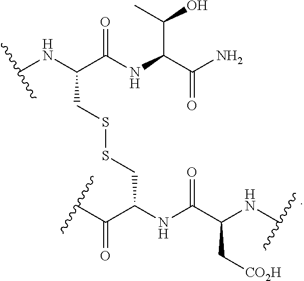 [SMILES: *N[C@@H](CC(=O)O)C(=O)N[C@@H](CSSC[C@H](N*)C(=O)N[C@H](C(N)=O)[C@@H](C)O)C(*)=O]

In some embodiments, L¹ is

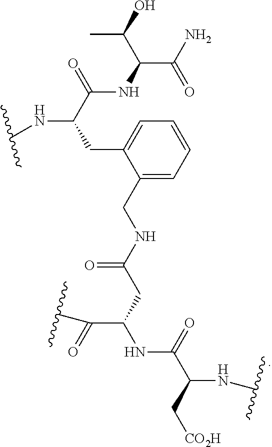 [SMILES: *N[C@@H](CC(=O)O)C(=O)N[C@@H](CC(=O)NCc1ccccc1C[C@H](N*)C(=O)N[C@H](C(N)=O)[C@@H](C)O)C(*)=O]

In some embodiments, L¹ is

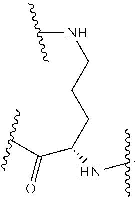 [SMILES: *NCCC[C@H](N*)C(*)=O]

In some embodiments, L¹ is

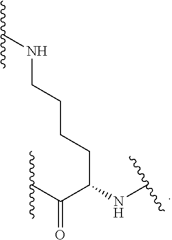 [SMILES: *NCCCC[C@H](N*)C(*)=O]

In some embodiments, L′ is

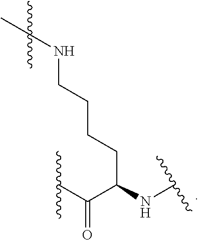 [SMILES: *NCCCC[C@@H](N*)C(*)=O]

In some embodiments, L¹ is

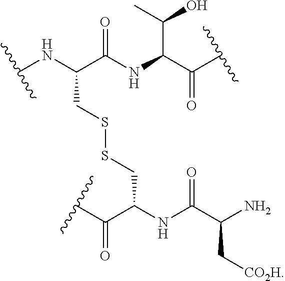 [SMILES: *N[C@@H](CSSC[C@H](NC(=O)[C@@H](N)CC(=O)O)C(*)=O)C(=O)N[C@H](C(*)=O)[C@@H](C)O]

In some embodiments, L¹ is

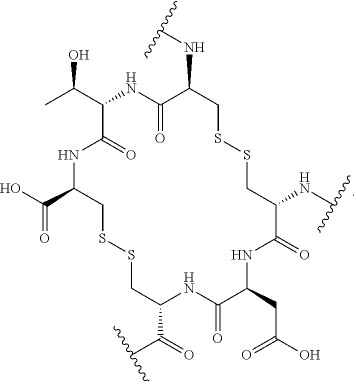 [SMILES: *N[C@H]1CSSC[C@H](N*)C(=O)N[C@@H]([C@@H](C)O)C(=O)N[C@H](C(=O)O)CSSC[C@@H](C(*)=O)NC(=O)[C@H](CC(=O)O)NC1=O]

In some embodiments, L¹ is

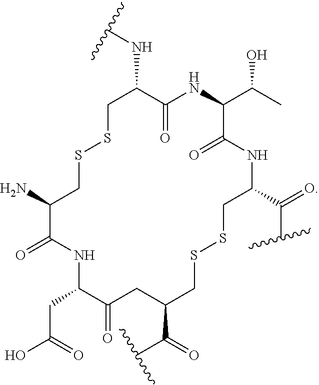 [SMILES: *N[C@H]1CSSC[C@H](N)C(=O)N[C@@H](CC(=O)O)C(=O)C[C@H](C(*)=O)CSSC[C@@H](C(*)=O)NC(=O)[C@H]([C@@H](C)O)NC1=O]

In some embodiments, L¹ is

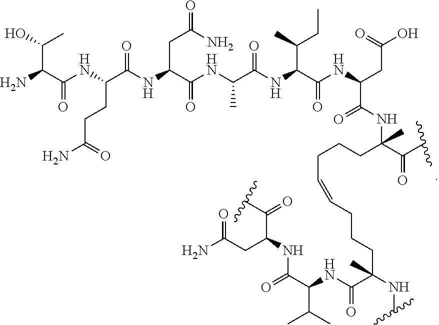 [SMILES: *N[C@@](C)(CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(*)=O)C(=O)N[C@H](C(=O)N[C@@H](CC(N)=O)C(*)=O)C(C)C]

In some embodiments, L¹ is

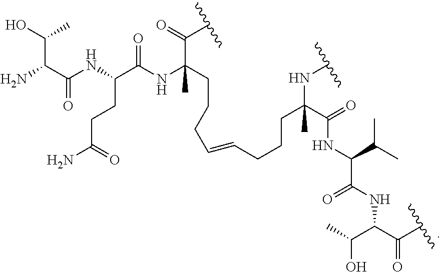 [SMILES: *N[C@@](C)(CCC/C=C/CCC[C@](C)(NC(=O)[C@H](CCC(N)=O)NC(=O)[C@H](N)[C@@H](C)O)C(*)=O)C(=O)N[C@H](C(=O)N[C@H](C(*)=O)[C@@H](C)O)C(C)C]

In some embodiments, L¹ is

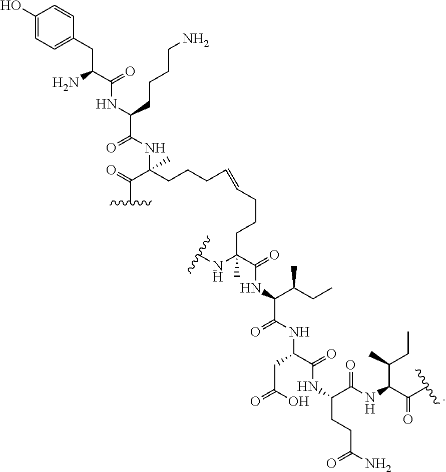 [SMILES: *N[C@@](C)(CCC/C=C/CCC[C@](C)(NC(=O)[C@H](CCCCN)NC(=O)[C@@H](N)Cc1ccc(O)cc1)C(*)=O)C(=O)N[C@H](C(=O)N[C@@H](CC(=O)O)C(=O)N[C@@H](CCC(N)=O)C(=O)N[C@H](C(*)=O)[C@@H](C)CC)[C@@H](C)CC]

In some embodiments, L¹ is

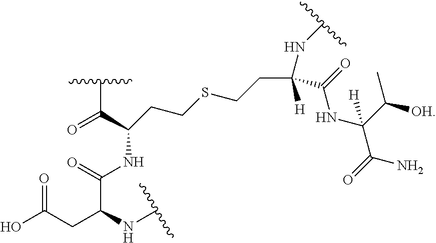

In some embodiments, L¹ is

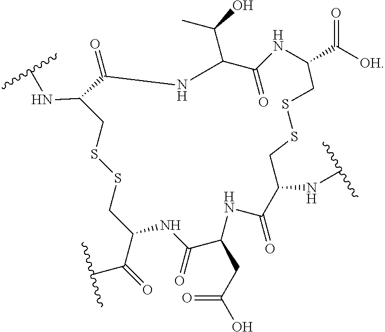 [SMILES: *N[C@H]1CSSC[C@@H](C(*)=O)NC(=O)[C@H](CC(=O)O)NC(=O)[C@@H](N*)CSSC[C@@H](C(=O)O)NC(=O)C([C@@H](C)O)NC1=O]

In some embodiments, L¹ is

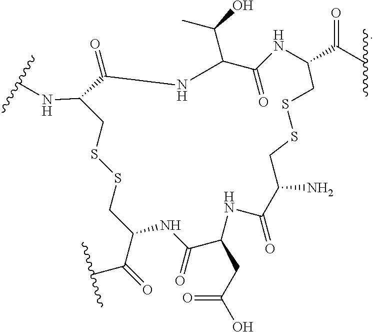 [SMILES: *N[C@H]1CSSC[C@@H](C(*)=O)NC(=O)[C@H](CC(=O)O)NC(=O)[C@@H](N)CSSC[C@@H](C(*)=O)NC(=O)C([C@@H](C)O)NC1=O]

In some embodiments, L¹ is

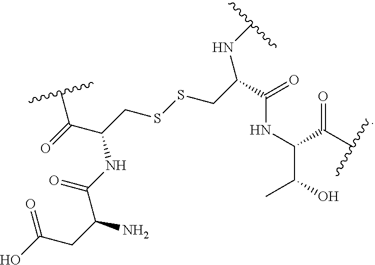 [SMILES: *N[C@@H](CSSC[C@H](NC(=O)[C@@H](N)CC(=O)O)C(*)=O)C(=O)N[C@H](C(*)=O)[C@@H](C)O]

In some embodiments, L¹ is selected from those depicted in Table 1, below.

As defined above and described herein, L² is a covalent bond or a C₁₋₁₀ bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—,

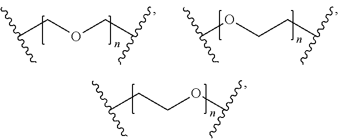

or -Cy¹-, wherein each -Cy- is independently a 5-6 membered heteroarylenyl with 1-4 heteroatoms independently selected from nitrogen, oxygen or sulfur.

In some embodiments, L² is a covalent bond. In some embodiments, L² is a C₁₋₁₀ bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—,

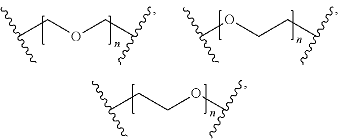

or -Cy-, wherein each -Cy- is independently a 5-6 membered heteroarylenyl with 1-4 heteroatoms independently selected from nitrogen, oxygen or sulfur.

In some embodiments, L² is

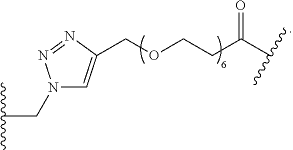

In some embodiments, L² is

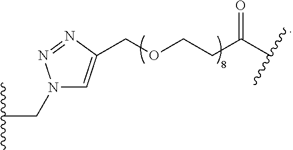

In some embodiments, L² is

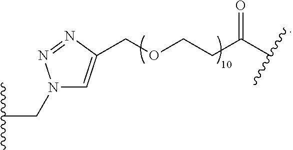

In some embodiments, L² is

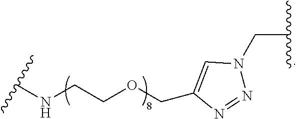

In some embodiments, L² is

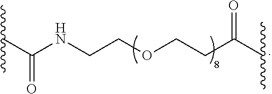

In some embodiments, L² is

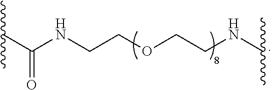

In some embodiments, L² is selected from those depicted in Table 1, below.

In some embodiments, L is L² as described in the present disclosure.

As defined above and described herein, TBT is a target binding moiety.

In some embodiments, TBT is a target binding moiety.

In some embodiments, TBT is

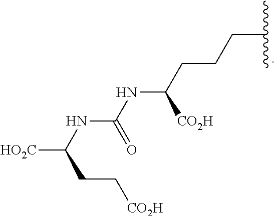 [SMILES: *CCC[C@H](NC(=O)N[C@@H](CCC(=O)O)C(=O)O)C(=O)O]

In some embodiments, TBT is

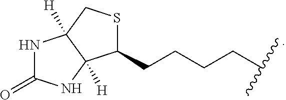 [SMILES: *CCCC[C@@H]1SC[C@@H]2NC(=O)N[C@@H]21]

In some embodiments, TBT is selected from those depicted in Table 1, below.

As defined above and described herein, each of m and n is independently 1, 2, 3, 4, 5, 6, 7, 8, 9, or 10.

In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

In some embodiments, m is selected from those depicted in Table 1, below.

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, n is selected from those depicted in Table 1, below.

As defined above and described herein, each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or: an R⁷ group and the R^{7′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur.

In some embodiments, R′ is hydrogen. In some embodiments, R′ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R⁷ is an optionally substituted C₁₋₆ aliphatic group. In some embodiments, R′ is an optionally substituted 3-8 membered saturated or partially unsaturated monocyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 4-8 membered saturated or partially unsaturated monocyclic heterocyclic ring having 1-2 heteroatoms independently selected from nitrogen, oxygen, or sulfur. In some embodiments, R⁷ is an optionally substituted 5-6 membered monocyclic heteroaromatic ring having 1-4 heteroatoms independently selected from nitrogen, oxygen, or sulfur. In some embodiments, R′ is an optionally substituted 8-10 membered bicyclic heteroaromatic ring having 1-5 heteroatoms independently selected from nitrogen, oxygen, or sulfur.

In some embodiments, R is methyl. In some embodiments, R⁷ is

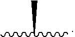

In some embodiments, R⁷ is

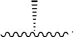

In some embodiments, R⁷ is

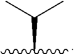

In some embodiments, R⁷ is

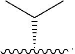

In some embodiments, R⁷ is

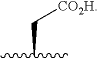

In some embodiments, R⁷ is

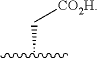

In some embodiments, R⁷ is

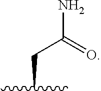

In some embodiments, R⁷ is

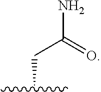

In some embodiments, R⁷ is

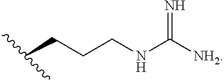

In some embodiments, R⁷ is

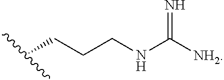

In some embodiments, R⁷ is

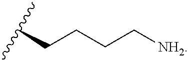

In some embodiments, R⁷ is

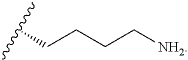

In some embodiments, R⁷ is

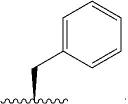

In some embodiments, R⁷ is

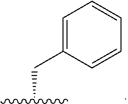

In some embodiments, R⁷ is

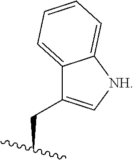

In some embodiments, R⁷ is

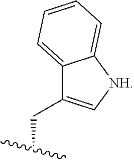

In some embodiments, R⁷ is

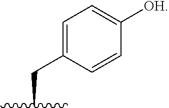

In some embodiments, R⁷ is

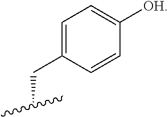

In some embodiments, R⁷ is

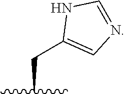

In some embodiments, R⁷ is

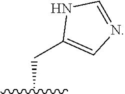

In some embodiments, an R⁷ group and the R^{7′} group attached to the same carbon atom are taken together with their intervening carbon atom to form a 3-8 membered saturated or partially unsaturated spirocyclic carbocyclic ring. In some embodiments, an R⁷ group and the R^{7′} group attached to the same carbon atom are taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R⁷ is selected from those depicted in Table 1, below.

As defined above and described herein, each of R^{7′} is independently hydrogen or C₁₋₃ aliphatic.

In some embodiments, R⁷ is hydrogen. In some embodiments, R^{7′} is methyl. In some embodiments, R⁷ is ethyl. In some embodiments, R^{7′} is n-propyl. In some embodiments, R^{7′} is isopropyl.

In some embodiments, R^{7′} is selected from those depicted in Table 1, below.

As defined above and described herein, each of R⁸ is independently hydrogen, or C₁₋₄ aliphatic, or: an R⁸ group and its adjacent R⁷ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R⁸ is hydrogen. In some embodiments, R⁸ is C₁₋₄ aliphatic. In some embodiments, R⁸ is methyl. In some embodiments, R⁸ is ethyl. In some embodiments, R⁸ is n-propyl. In some embodiments, R⁸ is isopropyl. In some embodiments, R⁸ is n-butyl. In some embodiments, R⁸ is isobutyl. In some embodiments, R⁸ is tert-butyl.

In some embodiments, an R⁸ group and its adjacent R⁷ group are taken together with their intervening atoms to form a 4-8 membered saturated or partially unsaturated monocyclic heterocyclic ring having 1-2 heteroatoms independently selected from nitrogen, oxygen, or sulfur.

In some embodiments, an R⁸ group and its adjacent R⁷ group are taken together with their intervening atoms to form

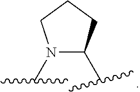 [SMILES: *[C@@H]1CCCN1*]

In some embodiments, an R⁸ group and its adjacent R⁷ group are taken together with their intervening atoms to form

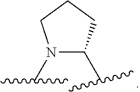 [SMILES: *[C@H]1CCCN1*]

In some embodiments, R⁸ is selected from those depicted in Table 1, below.

As defined above and described herein, R⁹ is hydrogen, C₁₋₃ aliphatic, or —C(O) C₁₋₃ aliphatic.

In some embodiments, R⁹ is hydrogen. In some embodiments, R⁹ is C₁₋₃ aliphatic. In some embodiments, R⁹ is —C(O) C₁₋₃ aliphatic.

In some embodiments, R⁹ is methyl. In some embodiments, R⁹ is ethyl. In some embodiments, R⁹ is n-propyl. In some embodiments, R⁹ is isopropyl. In some embodiments, R⁹ is cyclopropyl.

In some embodiments, R⁹ is —C(O) Me. In some embodiments, R⁹ is —C(O)Et. In some embodiments, R⁹ is —C(O) CH₂CH₂CH₃. In some embodiments, R⁹ is —C(O)CH(CH₃) 2. In some embodiments, R⁹ is —C(O)cyclopropyl.

In some embodiments, R⁹ is selected from those depicted in Table 1, below.

In some embodiments, R is R⁷ as described in the present disclosure. In some embodiments, R^{a2} is R⁷ as described in the present disclosure. In some embodiments, R^{a3} is R⁷ as described in the present disclosure. In some embodiments, R is R^{7′} as described in the present disclosure. In some embodiments, R^{a2} is R^{7′} as described in the present disclosure. In some embodiments, R^{a3} is R^{7′} as described in the present disclosure. In some embodiments, R is R⁸ as described in the present disclosure. In some embodiments, R^{a2} is R⁸ as described in the present disclosure. In some embodiments, R^{a3} is R⁸ as described in the present disclosure. In some embodiments, R is R^{8′} as described in the present disclosure. In some embodiments, R^{a2} is R^{8′} as described in the present disclosure. In some embodiments, R^{a3} is R⁸ as described in the present disclosure. In some embodiments, R is R⁹ as described in the present disclosure. In some embodiments, R^{a2} is R⁹ as described in the present disclosure. In some embodiments, R^{a3} is R⁹ as described in the present disclosure.

As defined above and described herein, L³ is a bivalent linker moiety that connects

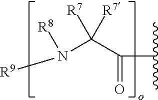

with TBT.

In some embodiments, L³ is a bivalent linker moiety that connects

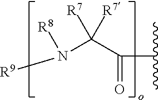

with TBT.

In some embodiments, L³ is

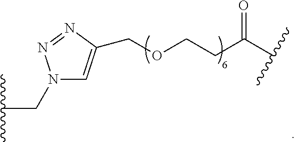

In some embodiments, L³ is

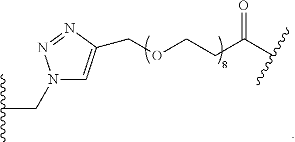

In some embodiments, L³ is

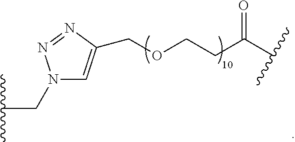

In some embodiments, L³ is

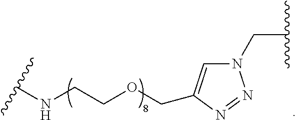

In some embodiments, L³ is

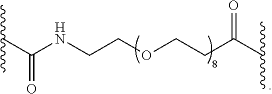

In some embodiments, L³ is

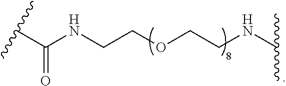

In some embodiments, L³ is selected from those depicted in Table 1, below.

In some embodiments, L is L³ as described in the present disclosure.

As defined above and described herein, o is 1, 2, 3, 4, 5, 6, 7, 8, 9, or 10.

In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8. In some embodiments, o is 9. In some embodiments, o is 10.

In some embodiments, o is selected from those depicted in Table 1, below.

In certain embodiments, the present invention provides a compound of formula II, wherein L² is

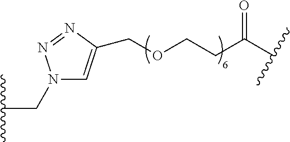

### and TBT is

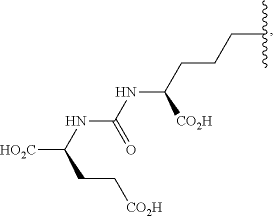 [SMILES: *CCC[C@H](NC(=O)N[C@@H](CCC(=O)O)C(=O)O)C(=O)O]

thereby forming a compound of formula II-a:

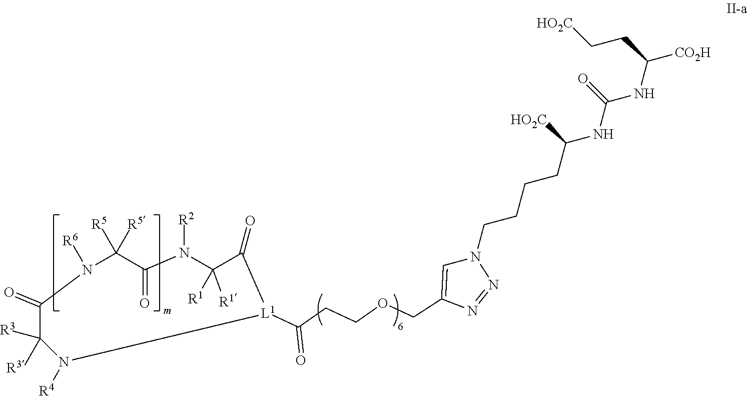

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present invention provides a compound of formula II, wherein L² is

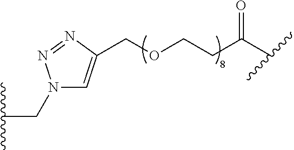

### and TBT is

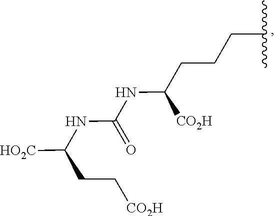 [SMILES: *CCC[C@H](NC(=O)N[C@@H](CCC(=O)O)C(=O)O)C(=O)O]

thereby forming a compound of formula II-b:

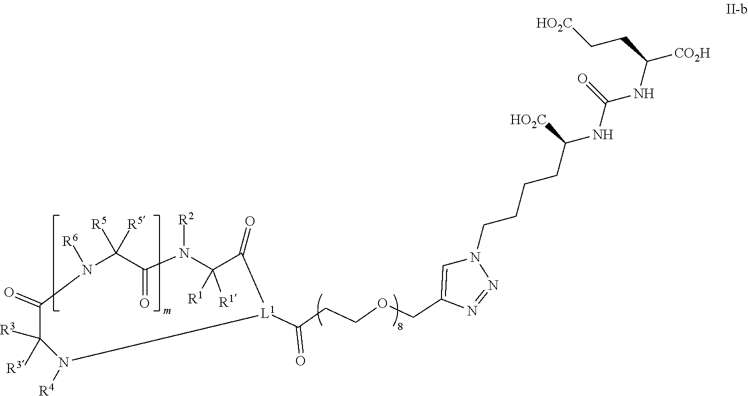

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present invention provides a compound of formula II, wherein L² is

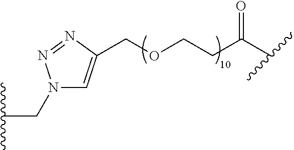

### and TBT is

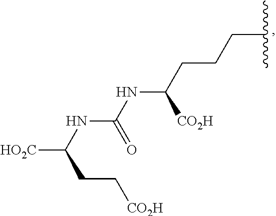 [SMILES: *CCC[C@H](NC(=O)N[C@@H](CCC(=O)O)C(=O)O)C(=O)O]

thereby forming a compound of formula II-c:

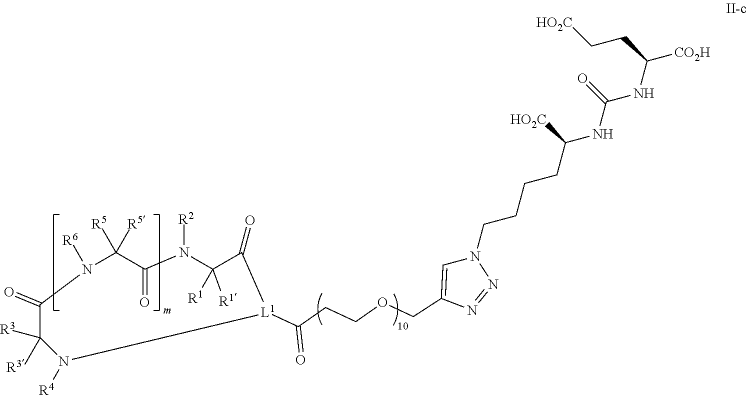

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present invention provides a compound of formula II, wherein L² is

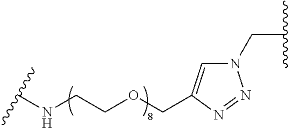

### and TBT is

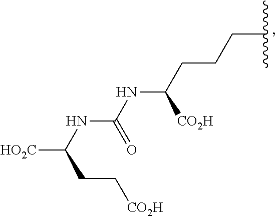 [SMILES: *CCC[C@H](NC(=O)N[C@@H](CCC(=O)O)C(=O)O)C(=O)O]

thereby forming a compound of formula II-d:

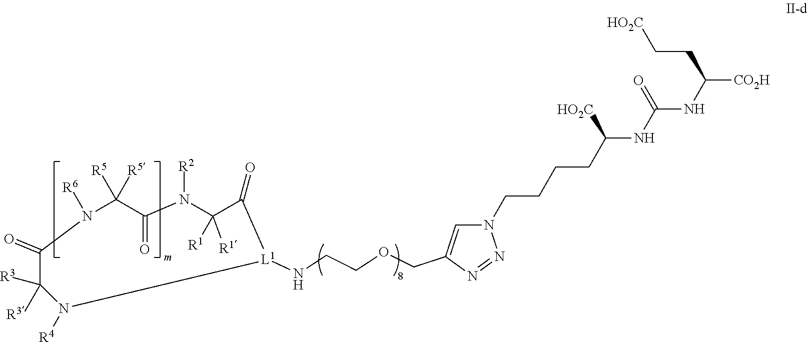

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present invention provides a compound of formula II, wherein L² is

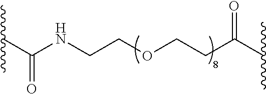

### and TBT is

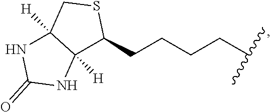 [SMILES: *CCCC[C@@H]1SC[C@@H]2NC(=O)N[C@@H]21]

thereby forming a compound of formula II-e:

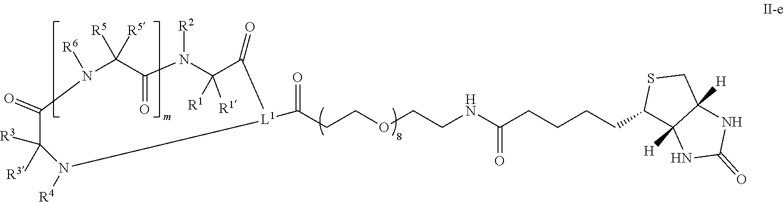

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In certain embodiments, the present invention provides a compound of formula II, wherein L² is

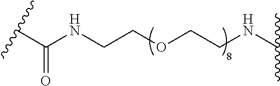

### and TBT is

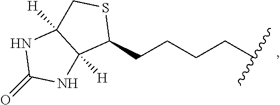 [SMILES: *CCCC[C@@H]1SC[C@@H]2NC(=O)N[C@@H]21]

thereby forming a compound of formula II-f:

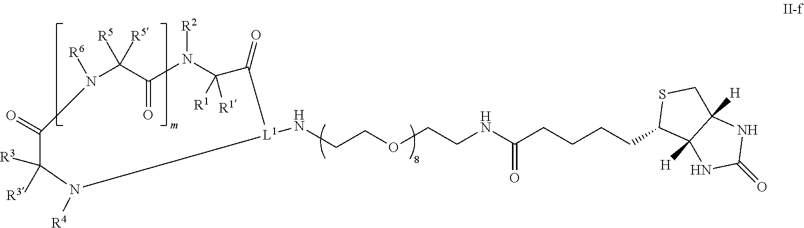

or a pharmaceutically acceptable salt thereof, wherein each of L¹, R¹, R^{1′}, R², R³, R^{3′}, R⁴, R⁵, R^{5′}, R⁶, and m is as defined above and described in embodiments herein, both singly and in combination.

In some embodiments, R^{a1} is R as described in the present disclosure. In some embodiments, R^{a1} is optionally substituted C₁₋₄ aliphatic.

In some embodiments, Lal is L^{a} as described in the present disclosure. In some embodiments, Lal is a covalent bond.

In some embodiments, L^{a2} is L^{a} as described in the present disclosure. In some embodiments, L^{a2} is a covalent bond.

In some embodiments, L^{a} is a covalent bond. In some embodiments, L^{a} is an optionally substituted bivalent group selected C₁-C₁₀ aliphatic or C₁-C₁₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is an optionally substituted bivalent group selected C₁-C₅ aliphatic or C₁-C₅ heteroaliphatic having 1-5 heteroatoms, wherein one or more methylene units of the group are optionally and independently replaced with —C(R)₂—, -Cy-, —O—, —S—, —S—S—, —N(R′)—, —C(O)—, —C(S)—, —C(NR′)—, —C(O)N(R)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is an optionally substituted bivalent C₁-C₅ aliphatic, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is an optionally substituted bivalent C₁-C₅ aliphatic. In some embodiments, L^{a} is an optionally substituted bivalent C₁-C₅ heteroaliphatic having 1-3 heteroatoms independently selected from nitrogen, oxygen and sulfur.

In some embodiments, R^{a2} is R as described in the present disclosure. In some embodiments, R^{a2} is a side chain of a natural amino acid. In some embodiments, R^{a3} is R as described in the present disclosure. In some embodiments, R^{a3} is a side chain of a natural amino acid. In some embodiments, one of R^{2a} and R^{3a} is hydrogen.

In some embodiments, each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon. In some embodiments, -Cy- is an optionally substituted ring as described in the present disclosure, for example, for R and Cy^{L}, but is bivalent.

In some embodiments, -Cy- is monocyclic. In some embodiments, -Cy- is bicyclic. In some embodiments, Cy- is polycyclic. In some embodiments, -Cy- is saturated. In some embodiments, -Cy- is partially unsaturated. In some embodiments, -Cy- is aromatic. In some embodiments, -Cy-comprises a saturated cyclic moiety. In some embodiments, -Cy-comprises a partially unsaturated cyclic moiety. In some embodiments, -Cy-comprises an aromatic cyclic moiety. In some embodiments, -Cy-comprises a combination of a saturated, a partially unsaturated, and/or an aromatic cyclic moiety. In some embodiments, -Cy- is 3-membered. In some embodiments, Cy- is 4-membered. In some embodiments, -Cy- is 5-membered. In some embodiments, -Cy- is 6-membered. In some embodiments, -Cy- is 7-membered. In some embodiments, -Cy- is 8-membered. In some embodiments, -Cy- is 9-membered. In some embodiments, -Cy- is 10-membered. In some embodiments, -Cy- is 11-membered. In some embodiments, -Cy- is 12-membered. In some embodiments, -Cy- is 13-membered. In some embodiments, -Cy- is 14-membered. In some embodiments, -Cy- is 15-membered. In some embodiments, -Cy- is 16-membered. In some embodiments, -Cy- is 17-membered. In some embodiments, -Cy- is 18-membered. In some embodiments, -Cy- is 19-membered. In some embodiments, -Cy- is 20-membered.

In some embodiments, -Cy- is an optionally substituted bivalent C3.20 cycloaliphatic ring. In some embodiments, Cy- is an optionally substituted bivalent, saturated C₃₋₂₀ cycloaliphatic ring. In some embodiments, Cy- is an optionally substituted bivalent, partially unsaturated C₃₋₂₀ cycloaliphatic ring. In some embodiments, Cy-H is optionally substituted cycloaliphatic as described in the present disclosure, for example, cycloaliphatic embodiments for R.

In some embodiments, -Cy- is an optionally substituted C₆₋₂₀ aryl ring. In some embodiments, -Cy- is optionally substituted phenylene. In some embodiments, -Cy- is optionally substituted 1,2-phenylene. In some embodiments, -Cy- is optionally substituted 1,3-phenylene. In some embodiments, -Cy- is optionally substituted 1,4-phenylene. In some embodiments, -Cy- is an optionally substituted bivalent naphthalene ring. In some embodiments, -Cy-H is optionally substituted aryl as described in the present disclosure, for example, aryl embodiments for R.

In some embodiments, -Cy- is an optionally substituted bivalent 5-20 membered heteroaryl ring having 1-10 heteroatoms independently selected from oxygen, nitrogen, sulfur, phosphorus and silicon. In some embodiments, -Cy- is an optionally substituted bivalent 5-20 membered heteroaryl ring having 1-10 heteroatoms independently selected from oxygen, nitrogen, and sulfur. In some embodiments, -Cy- is an optionally substituted bivalent 5-6 membered heteroaryl ring having 1-4 heteroatoms independently selected from oxygen, nitrogen, sulfur. In some embodiments, -Cy- is an optionally substituted bivalent 5-6 membered heteroaryl ring having 1-3 heteroatoms independently selected from oxygen, nitrogen, sulfur. In some embodiments, -Cy- is an optionally substituted bivalent 5-6 membered heteroaryl ring having 1-2 heteroatoms independently selected from oxygen, nitrogen, sulfur. In some embodiments, -Cy- is an optionally substituted bivalent 5-6 membered heteroaryl ring having one heteroatom independently selected from oxygen, nitrogen, sulfur. In some embodiments, -Cy-H is optionally substituted heteroaryl as described in the present disclosure, for example, heteroaryl embodiments for R. In some embodiments, -Cy- is

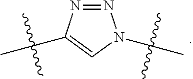 [SMILES: *c1cn(*)nn1]

In some embodiments, -Cy- is an optionally substituted bivalent 3-20 membered heterocyclyl ring having 1-10 heteroatoms independently selected from oxygen, nitrogen, sulfur, phosphorus and silicon. In some embodiments, -Cy- is an optionally substituted bivalent 3-20 membered heterocyclyl ring having 1-10 heteroatoms independently selected from oxygen, nitrogen, and sulfur. In some embodiments, -Cy- is an optionally substituted bivalent 3-6 membered heterocyclyl ring having 1-4 heteroatoms independently selected from oxygen, nitrogen, sulfur. In some embodiments, -Cy- is an optionally substituted bivalent 5-6 membered heterocyclyl ring having 1-4 heteroatoms independently selected from oxygen, nitrogen, sulfur. In some embodiments, -Cy- is an optionally substituted bivalent 5-6 membered heterocyclyl ring having 1-3 heteroatoms independently selected from oxygen, nitrogen, sulfur. In some embodiments, -Cy- is an optionally substituted bivalent 5-6 membered heterocyclyl ring having 1-2 heteroatoms independently selected from oxygen, nitrogen, sulfur. In some embodiments, Cy- is an optionally substituted bivalent 5-6 membered heterocyclyl ring having one heteroatom independently selected from oxygen, nitrogen, sulfur. In some embodiments, -Cy- is an optionally substituted saturated bivalent heterocyclyl group. In some embodiments, -Cy- is an optionally substituted partially unsaturated bivalent heterocyclyl group. In some embodiments, -Cy-H is optionally substituted heterocyclyl as described in the present disclosure, for example, heterocyclyl embodiments for R. In some embodiments, each Xaa is independently an amino acid residue. In some embodiments, each Xaa is independently an amino acid residue of an amino acid of formula A-I. In some embodiments, t is 0. In some embodiments, t is 1-50. In some embodiments, t is z as described in the present disclosure.

In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10. In some embodiments, z is 11. In some embodiments, z is 12. In some embodiments, z is 13. In some embodiments, z is 14. In some embodiments, z is 15. In some embodiments, z is 16. In some embodiments, z is 17. In some embodiments, z is 18. In some embodiments, z is 19. In some embodiments, z is 20. In some embodiments, z is greater than 20.

In some embodiments, R^{c} is R′ as described in the present disclosure. In some embodiments, R^{e} is R as described in the present disclosure. In some embodiments, R^{c} is —N(R′)₂, wherein each R′ is independently as described in the present disclosure. In some embodiments, R^{c} is —NH₂. In some embodiments, R^{c} is R—C(O)—, wherein R is as described in the present disclosure.

In some embodiments, a is 1. In some embodiments, a is 2-100. In some embodiments, a is 5. In some embodiments, a is 10. In some embodiments, a is 20. In some embodiments, a is 50.

In some embodiments, b is 1. In some embodiments, b is 2-100. In some embodiments, b is 5. In some embodiments, b is 10. In some embodiments, b is 20. In some embodiments, b is 50.

In some embodiments, a1 is 0. In some embodiments, a1 is 1.

In some embodiments, a2 is 0. In some embodiments, a2 is 1.

In some embodiments, L^{b} is L^{a} as described in the present disclosure. In some embodiments, L^{b} comprises -Cy-. In some embodiments, L^{b} comprises a double bond. In some embodiments, L^{b} comprises —S—. In some embodiments, L^{b} comprises —S—S—. In some embodiments, L^{b} comprises —C(O)—N(R′)—.

In some embodiments, R′ is —R, —C(O)R, —C(O)OR, or —S(O)₂R, wherein R is as described in the present disclosure. In some embodiments, R′ is R, wherein R is as described in the present disclosure. In some embodiments, R′ is —C(O)R, wherein R is as described in the present disclosure. In some embodiments, R′ is —C(O)OR, wherein R is as described in the present disclosure. In some embodiments, R′ is —S(O)₂R, wherein R is as described in the present disclosure. In some embodiments, R′ is hydrogen. In some embodiments, R′ is not hydrogen. In some embodiments, R′ is R, wherein R is optionally substituted C₁₋₂₀ aliphatic as described in the present disclosure. In some embodiments, R′ is R, wherein R is optionally substituted C₁₋₂₀ heteroaliphatic as described in the present disclosure. In some embodiments, R′ is R, wherein R is optionally substituted C₆₋₂₀ aryl as described in the present disclosure. In some embodiments, R′ is R, wherein R is optionally substituted C₆₋₂₀ arylaliphatic as described in the present disclosure. In some embodiments, R′ is R, wherein R is optionally substituted C₆₋₂₀ arylheteroaliphatic as described in the present disclosure. In some embodiments, R′ is R, wherein R is optionally substituted 5-20 membered heteroaryl as described in the present disclosure. In some embodiments, R′ is R, wherein R is optionally substituted 3-20 membered heterocyclyl as described in the present disclosure. In some embodiments, two or more R′ are R, and are optionally and independently taken together to form an optionally substituted ring as described in the present disclosure.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon.
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon.
two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon.

In some embodiments, R is hydrogen. In some embodiments, R is not hydrogen. In some embodiments, R is an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, a 5-30 membered heteroaryl ring having 1-10 heteroatoms independently selected from oxygen, nitrogen, sulfur, phosphorus and silicon, and a 3-30 membered heterocyclic ring having 1-10 heteroatoms independently selected from oxygen, nitrogen, sulfur, phosphorus and silicon.

In some embodiments, R is hydrogen or an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, and sulfur, a 4-7 membered saturated or partially unsaturated heterocyclic ring having 1-3 heteroatoms independently selected from nitrogen, oxygen, and sulfur, a 7-10 membered bicyclic saturated or partially unsaturated heterocyclic ring having 1-5 heteroatoms independently selected from nitrogen, oxygen, and sulfur, or an 8-10 membered bicyclic heteroaryl ring having 1-5 heteroatoms independently selected from nitrogen, oxygen, and sulfur.

In some embodiments, R is optionally substituted C₁₋₃₀ aliphatic. In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C16 aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl. In some embodiments, R is —(CH₂)₂CN.

In some embodiments, R is optionally substituted C₃₋₃₀ cycloaliphatic. In some embodiments, R is optionally substituted C₃₋₂₀ cycloaliphatic. In some embodiments, R is optionally substituted C₃₋₁₀ cycloaliphatic. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, R is an optionally substituted 3-30 membered saturated or partially unsaturated carbocyclic ring. In some embodiments, R is an optionally substituted 3-7 membered saturated or partially unsaturated carbocyclic ring. In some embodiments, R is an optionally substituted 3-membered saturated or partially unsaturated carbocyclic ring. In some embodiments, R is an optionally substituted 4-membered saturated or partially unsaturated carbocyclic ring. In some embodiments, R is an optionally substituted 5-membered saturated or partially unsaturated carbocyclic ring. In some embodiments, R is an optionally substituted 6-membered saturated or partially unsaturated carbocyclic ring. In some embodiments, R is an optionally substituted 7-membered saturated or partially unsaturated carbocyclic ring. In some embodiments, R is optionally substituted cycloheptyl. In some embodiments, R is cycloheptyl. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, when R is or comprises a ring structure, e.g., cycloaliphatic, cycloheteroaliphatic, aryl, heteroaryl, etc., the ring structure can be monocyclic, bicyclic or polycyclic. In some embodiments, R is or comprises a monocyclic structure. In some embodiments, R is or comprises a bicyclic structure. In some embodiments, R is or comprises a polycyclic structure.

In some embodiments, R is optionally substituted C₁₋₃₀ heteroaliphatic having 1-10 heteroatoms independently selected from oxygen, nitrogen, sulfur, phosphorus and silicon. In some embodiments, R is optionally substituted C₁₋₂₀ heteroaliphatic having 1-10 heteroatoms. In some embodiments, R is optionally substituted C₁₋₂₀ heteroaliphatic having 1-10 heteroatoms independently selected from oxygen, nitrogen, sulfur, phosphorus or silicon, optionally including one or more oxidized forms of nitrogen, sulfur, phosphorus or selenium. In some embodiments, R is optionally substituted C₁₋₃₀ heteroaliphatic comprising 1-10 groups independently selected from

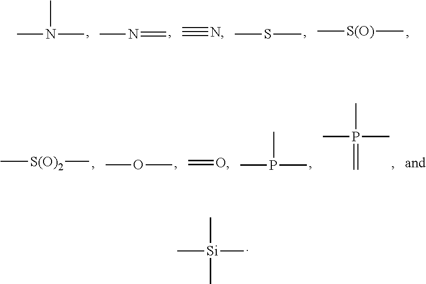

In some embodiments, R is optionally substituted C₆₋₃₀ aryl. In some embodiments, R is optionally substituted phenyl. In some embodiments, R is phenyl. In some embodiments, R is substituted phenyl.

In some embodiments, R is an optionally substituted 8-10 membered bicyclic saturated, partially unsaturated or aryl ring. In some embodiments, R is an optionally substituted 8-10 membered bicyclic saturated ring. In some embodiments, R is an optionally substituted 8-10 membered bicyclic partially unsaturated ring. In some embodiments, R is an optionally substituted 8-10 membered bicyclic aryl ring. In some embodiments, R is optionally substituted naphthyl.

In some embodiments, R is optionally substituted 5-30 membered heteroaryl ring having 1-10 heteroatoms independently selected from oxygen, nitrogen, sulfur, phosphorus and silicon. In some embodiments, R is optionally substituted 5-30 membered heteroaryl ring having 1-10 heteroatoms independently selected from oxygen, nitrogen, and sulfur. In some embodiments, R is optionally substituted 5-30 membered heteroaryl ring having 1-5 heteroatoms independently selected from oxygen, nitrogen, sulfur, phosphorus and silicon. In some embodiments, R is optionally substituted 5-30 membered heteroaryl ring having 1-5 heteroatoms independently selected from oxygen, nitrogen, and sulfur.

In some embodiments, R is an optionally substituted 5-6 membered monocyclic heteroaryl ring having 1-4 heteroatoms independently selected from nitrogen, oxygen, and sulfur. In some embodiments, R is a substituted 5-6 membered monocyclic heteroaryl ring having 1-4 heteroatoms independently selected from nitrogen, oxygen, and sulfur. In some embodiments, R is an unsubstituted 5-6 membered monocyclic heteroaryl ring having 1-4 heteroatoms independently selected from nitrogen, oxygen, and sulfur. In some embodiments, R is an optionally substituted 5-6 membered monocyclic heteroaryl ring having 1-3 heteroatoms independently selected from nitrogen, sulfur, and oxygen. In some embodiments, R is a substituted 5-6 membered monocyclic heteroaryl ring having 1-3 heteroatoms independently selected from nitrogen, oxygen, and sulfur. In some embodiments, R is an unsubstituted 5-6 membered monocyclic heteroaryl ring having 1-3 heteroatoms independently selected from nitrogen, sulfur, and oxygen.

In some embodiments, R is an optionally substituted 5-membered monocyclic heteroaryl ring having 1-4 heteroatoms independently selected from nitrogen, oxygen or sulfur. In some embodiments, R is an optionally substituted 6-membered monocyclic heteroaryl ring having 1-4 heteroatoms independently selected from nitrogen, oxygen, and sulfur. In some embodiments, R is an optionally substituted 5-membered monocyclic heteroaryl ring having one heteroatom selected from nitrogen, oxygen, and sulfur. In some embodiments, R is optionally substituted pyrrolyl, furanyl, or thienyl.

In some embodiments, R is an optionally substituted 5-membered heteroaryl ring having two heteroatoms independently selected from nitrogen, oxygen, and sulfur. In certain embodiments, R is an optionally substituted 5-membered heteroaryl ring having one nitrogen atom, and an additional heteroatom selected from sulfur or oxygen. In some embodiments, R is an optionally substituted 5-membered heteroaryl ring having three heteroatoms independently selected from nitrogen, oxygen, and sulfur. In some embodiments, R is an optionally substituted 5-membered heteroaryl ring having four heteroatoms independently selected from nitrogen, oxygen, and sulfur.

In some embodiments, R is an optionally substituted 6-membered heteroaryl ring having 1-4 nitrogen atoms. In some embodiments, R is an optionally substituted 6-membered heteroaryl ring having 1-3 nitrogen atoms. In other embodiments, R is an optionally substituted 6-membered heteroaryl ring having 1-2 nitrogen atoms. In some embodiments, R is an optionally substituted 6-membered heteroaryl ring having four nitrogen atoms. In some embodiments, R is an optionally substituted 6-membered heteroaryl ring having three nitrogen atoms. In some embodiments, R is an optionally substituted 6-membered heteroaryl ring having two nitrogen atoms. In certain embodiments, R is an optionally substituted 6-membered heteroaryl ring having one nitrogen atom.

In certain embodiments, R is an optionally substituted 8-10 membered bicyclic heteroaryl ring having 1-4 heteroatoms independently selected from nitrogen, oxygen, and sulfur. In some embodiments, R is an optionally substituted 5,6-fused heteroaryl ring having 1-4 heteroatoms independently selected from nitrogen, oxygen, and sulfur. In certain embodiments, R is an optionally substituted 6,6-fused heteroaryl ring having 1-4 heteroatoms independently selected from nitrogen, oxygen, and sulfur.

In some embodiments, R is 3-30 membered heterocyclic ring having 1-10 heteroatoms independently selected from oxygen, nitrogen, sulfur, phosphorus and silicon. In some embodiments, R is 3-30 membered heterocyclic ring having 1-10 heteroatoms independently selected from oxygen, nitrogen, and sulfur. In some embodiments, R is 3-30 membered heterocyclic ring having 1-5 heteroatoms independently selected from oxygen, nitrogen, sulfur, phosphorus and silicon. In some embodiments, R is 3-30 membered heterocyclic ring having 1-5 heteroatoms independently selected from oxygen, nitrogen, and sulfur.

In some embodiments, R is an optionally substituted 3-7 membered saturated or partially unsaturated heterocyclic ring having 1-3 heteroatoms independently selected from nitrogen, oxygen, and sulfur. In some embodiments, R is a substituted 3-7 membered saturated or partially unsaturated heterocyclic ring having 1-3 heteroatoms independently selected from nitrogen, oxygen, and sulfur. In some embodiments, R is an unsubstituted 3-7 membered saturated or partially unsaturated heterocyclic ring having 1-3 heteroatoms independently selected from nitrogen, oxygen, and sulfur. In certain embodiments, R is an optionally substituted 5-7 membered partially unsaturated monocyclic ring having 1-3 heteroatoms independently selected from nitrogen, oxygen, and sulfur. In certain embodiments, R is an optionally substituted 5-6 membered partially unsaturated monocyclic ring having 1-3 heteroatoms independently selected from nitrogen, oxygen, and sulfur. In certain embodiments, R is an optionally substituted 5-membered partially unsaturated monocyclic ring having 1-3 heteroatoms independently selected from nitrogen, oxygen, and sulfur. In certain embodiments, R is an optionally substituted 6-membered partially unsaturated monocyclic ring having 1-3 heteroatoms independently selected from nitrogen, oxygen, and sulfur. In certain embodiments, R is an optionally substituted 7-membered partially unsaturated monocyclic ring having 1-3 heteroatoms independently selected from nitrogen, oxygen, and sulfur. In some embodiments, R is optionally substituted 3-membered heterocyclic ring having one heteroatom selected from nitrogen, oxygen or sulfur. In some embodiments, R is optionally substituted 4-membered heterocyclic ring having 1-3 heteroatoms independently selected from nitrogen, oxygen, and sulfur. In some embodiments, R is optionally substituted 5-membered heterocyclic ring having 1-3 heteroatoms independently selected from nitrogen, oxygen, and sulfur. In some embodiments, R is optionally substituted 6-membered heterocyclic ring having 1-3 heteroatoms independently selected from nitrogen, oxygen, and sulfur. In some embodiments, R is optionally substituted 7-membered heterocyclic ring having 1-3 heteroatoms independently selected from nitrogen, oxygen, and sulfur.

In some embodiments, R is an optionally substituted 3-membered saturated or partially unsaturated heterocyclic ring having 1-2 heteroatoms independently selected from nitrogen, oxygen, and sulfur. In some embodiments, R is an optionally substituted 4-membered saturated or partially unsaturated heterocyclic ring having 1-2 heteroatoms independently selected from nitrogen, oxygen, and sulfur. In some embodiments, R is an optionally substituted 5-membered saturated or partially unsaturated heterocyclic ring having 1-2 heteroatoms independently selected from nitrogen, oxygen, and sulfur. In some embodiments, R is an optionally substituted 6-membered saturated or partially unsaturated heterocyclic ring having 1-2 heteroatoms independently selected from nitrogen, oxygen, and sulfur. In some embodiments, R is an optionally substituted 7-membered saturated or partially unsaturated heterocyclic ring having 1-2 heteroatoms independently selected from nitrogen, oxygen, and sulfur.

In certain embodiments, R is an optionally substituted 5-6 membered partially unsaturated monocyclic ring having 1-2 heteroatoms independently selected from nitrogen, oxygen, and sulfur. In certain embodiments, R is an optionally substituted tetrahydropyridinyl, dihydrothiazolyl, dihydrooxazolyl, or oxazolinyl group.

In some embodiments, R is an optionally substituted 7-10 membered bicyclic saturated or partially unsaturated heterocyclic ring having 1-5 heteroatoms independently selected from nitrogen, oxygen, and sulfur. In some embodiments, R is optionally substituted indolinyl. In some embodiments, R is optionally substituted isoindolinyl. In some embodiments, R is optionally substituted 1, 2, 3, 4-tetrahydroquinolinyl. In some embodiments, R is optionally substituted 1, 2, 3, 4-tetrahydroisoquinolinyl. In some embodiments, R is an optionally substituted azabicyclo[3.2.1]octanyl.

In some embodiments, R is an optionally substituted 8-10 membered bicyclic heteroaryl ring having 1-5 heteroatoms independently selected from nitrogen, oxygen, and sulfur. In some embodiments, R is an optionally substituted 5,6-fused heteroaryl ring having 1-5 heteroatoms independently selected from nitrogen, oxygen, and sulfur.

In some embodiments, R is optionally substituted C₆₋₃₀ arylaliphatic. In some embodiments, R is optionally substituted C₆₋₂₀ arylaliphatic. In some embodiments, R is optionally substituted C₆₋₁₀ arylaliphatic. In some embodiments, an aryl moiety of the arylaliphatic has 6, 10, or 14 aryl carbon atoms. In some embodiments, an aryl moiety of the arylaliphatic has 6 aryl carbon atoms. In some embodiments, an aryl moiety of the arylaliphatic has 10 aryl carbon atoms. In some embodiments, an aryl moiety of the arylaliphatic has 14 aryl carbon atoms. In some embodiments, an aryl moiety is optionally substituted phenyl.

In some embodiments, R is optionally substituted C₆₋₃₀ arylheteroaliphatic having 1-10 heteroatoms independently selected from oxygen, nitrogen, sulfur, phosphorus and silicon. In some embodiments, R is optionally substituted C₆₋₃₀ arylheteroaliphatic having 1-10 heteroatoms independently selected from oxygen, nitrogen, and sulfur. In some embodiments, R is optionally substituted C₆₋₂₀ arylheteroaliphatic having 1-10 heteroatoms independently selected from oxygen, nitrogen, sulfur, phosphorus and silicon. In some embodiments, R is optionally substituted C₆₋₂₀ arylheteroaliphatic having 1-10 heteroatoms independently selected from oxygen, nitrogen, and sulfur. In some embodiments, R is optionally substituted C₆₋₁₀ arylheteroaliphatic having 1-5 heteroatoms independently selected from oxygen, nitrogen, sulfur, phosphorus and silicon. In some embodiments, R is optionally substituted C₆₋₁₀ arylheteroaliphatic having 1-5 heteroatoms independently selected from oxygen, nitrogen, and sulfur.

In some embodiments, two R groups are optionally and independently taken together to form a covalent bond. In some embodiments, —C═O is formed. In some embodiments, —C═C— is formed. In some embodiments, —C≡C— is formed.

In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-10 membered monocyclic, bicyclic or polycyclic ring having, in addition to the atom, 0-5 heteroatoms independently selected from oxygen, nitrogen, sulfur, phosphorus and silicon. In some embodiments, two or more R groups on the same atom are optionally and independently taken together with the atom to form an optionally substituted, 3-6 membered monocyclic, bicyclic or polycyclic ring having, in addition to the atom, 0-3 heteroatoms independently selected from oxygen, nitrogen, sulfur, phosphorus and silicon. In some embodiments, two or more R groups on the same atom are optionally and independently taken together with the atom to form an optionally substituted, 3-5 membered monocyclic, bicyclic or polycyclic ring having, in addition to the atom, 0-3 heteroatoms independently selected from oxygen, nitrogen, sulfur, phosphorus and silicon.

In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-5 heteroatoms independently selected from oxygen, nitrogen, sulfur, phosphorus and silicon. In some embodiments, two or more R groups on two or more atoms are optionally and independently taken together with their intervening atoms to form an optionally substituted, 3-6 membered monocyclic, bicyclic or polycyclic ring having, in addition to the intervening atoms, 0-3 heteroatoms independently selected from oxygen, nitrogen, sulfur, phosphorus and silicon. In some embodiments, two or more R groups on two or more atoms are optionally and independently taken together with their intervening atoms to form an optionally substituted, 3-5 membered monocyclic, bicyclic or polycyclic ring having, in addition to the intervening atoms, 0-3 heteroatoms independently selected from oxygen, nitrogen, sulfur, phosphorus and silicon.

In some embodiments, heteroatoms in R groups, or in the structures formed by two or more R groups taken together, are selected from oxygen, nitrogen, and sulfur. In some embodiments, a formed ring is 3, 4, 5, 6, 7, 8, 9, 10, 11, 12, 13, 14, 15, 16, 17, 18, 19, or 20-membered. In some embodiments, a formed ring is saturated. In some embodiments, a formed ring is partially saturated. In some embodiments, a formed ring is aromatic. In some embodiments, a formed ring comprises a saturated, partially saturated, or aromatic ring moiety. In some embodiments, a formed ring comprises 5, 6, 7, 8, 9, 10, 11, 12, 13, 14, 15, 16, 17, 18, 19, or 20 aromatic ring atoms. In some embodiments, a formed contains no more than 5, 6, 7, 8, 9, 10, 11, 12, 13, 14, 15, 16, 17, 18, 19, or 20 aromatic ring atoms. In some embodiments, aromatic ring atoms are selected from carbon, nitrogen, oxygen and sulfur.

In some embodiments, a ring formed by two or more R groups (or two or more groups selected from R and variables that can be R) taken together is a C₃₋₃₀ cycloaliphatic, C₆₋₃₀ aryl, 5-30 membered heteroaryl having 1-10 heteroatoms independently selected from oxygen, nitrogen, sulfur, phosphorus and silicon, or 3-30 membered heterocyclyl having 1-10 heteroatoms independently selected from oxygen, nitrogen, sulfur, phosphorus and silicon, ring as described for R, but bivalent or multivalent.

Exemplary compounds of the invention are set forth in Table 1, below.

TABLE 1
Exemplary compounds

| | |
|---|---|
| 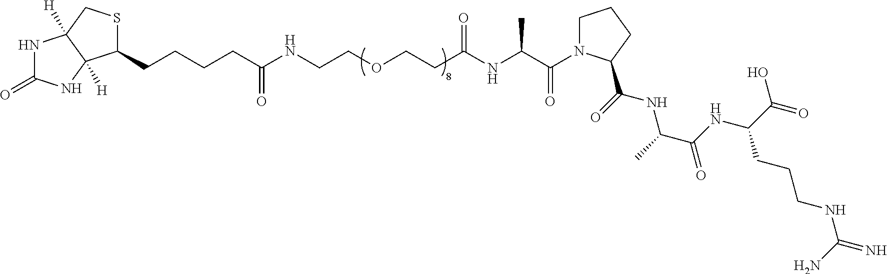 | I-1 |
| 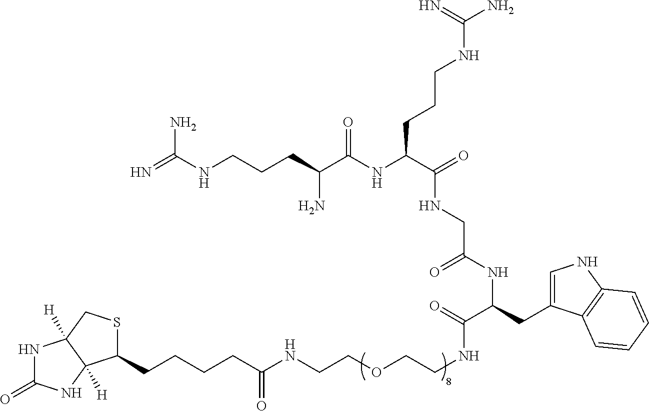 | I-2 |
| 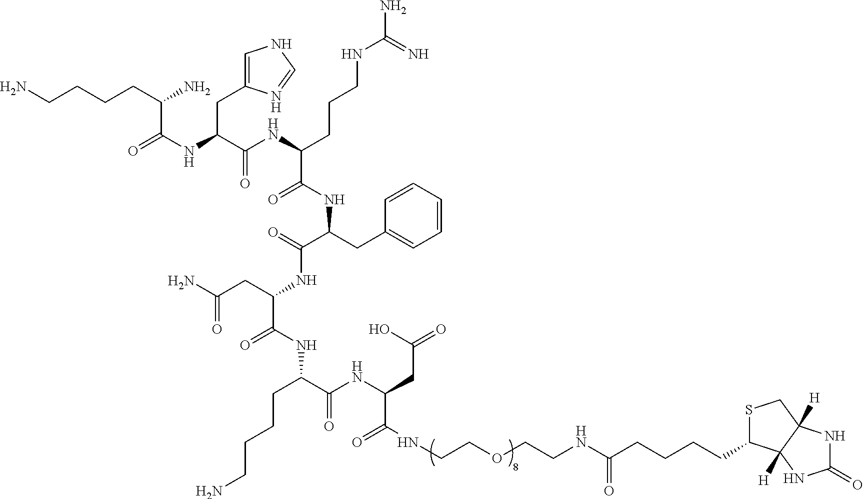 | I-3 |
| 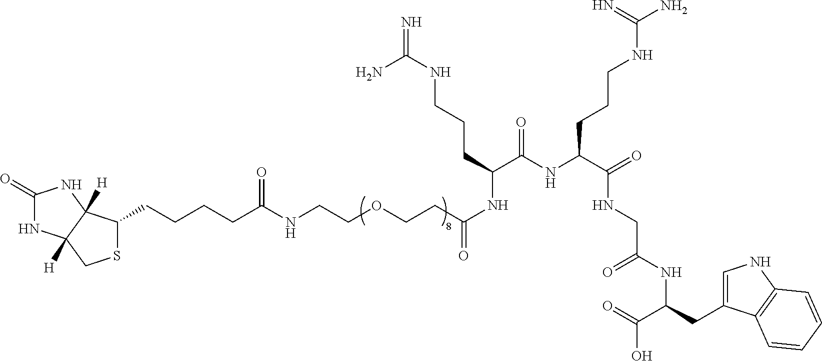 | I-4 |
| 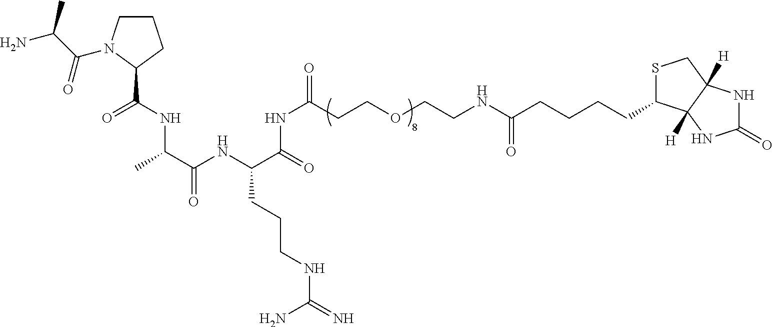 | I-5 |
| 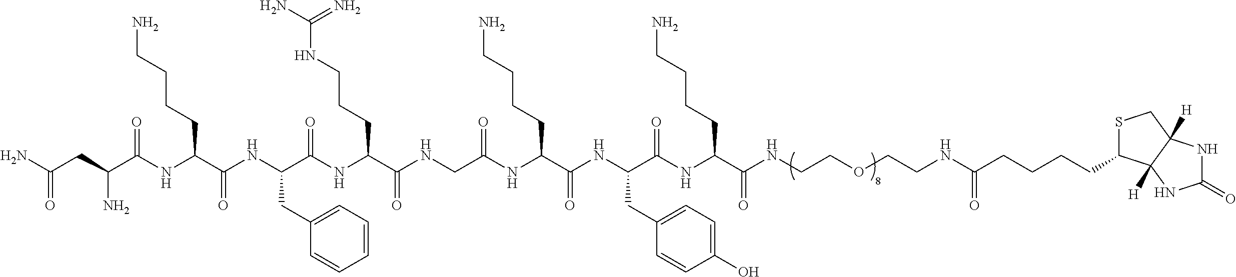 | I-6 |
| 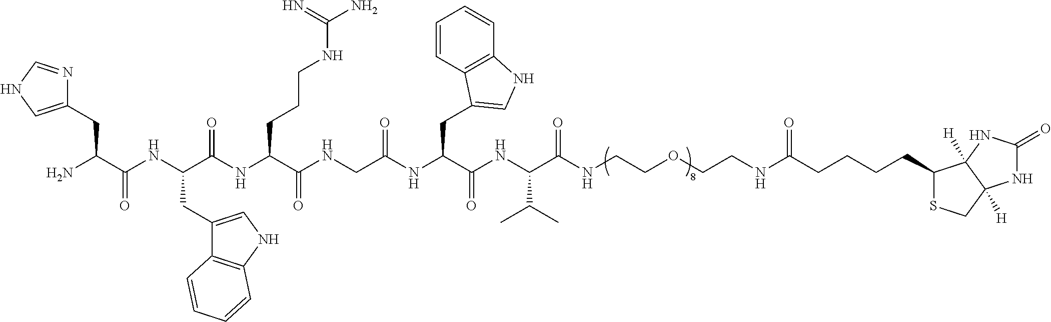 | I-7 |
| 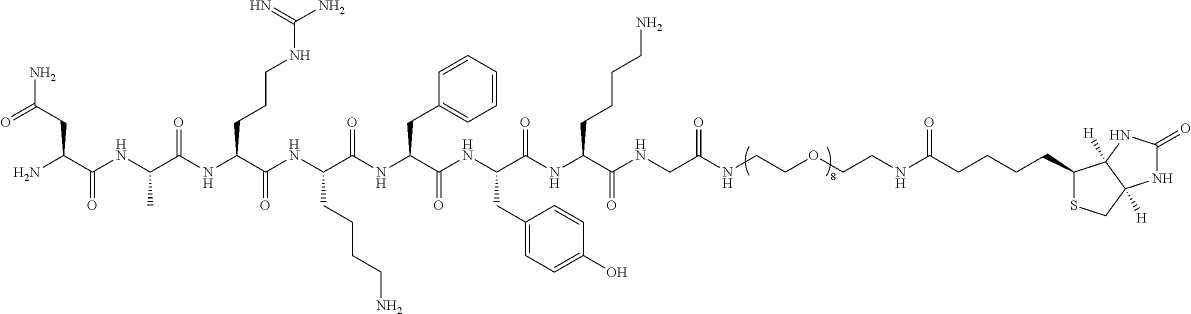 | I-8 |
| 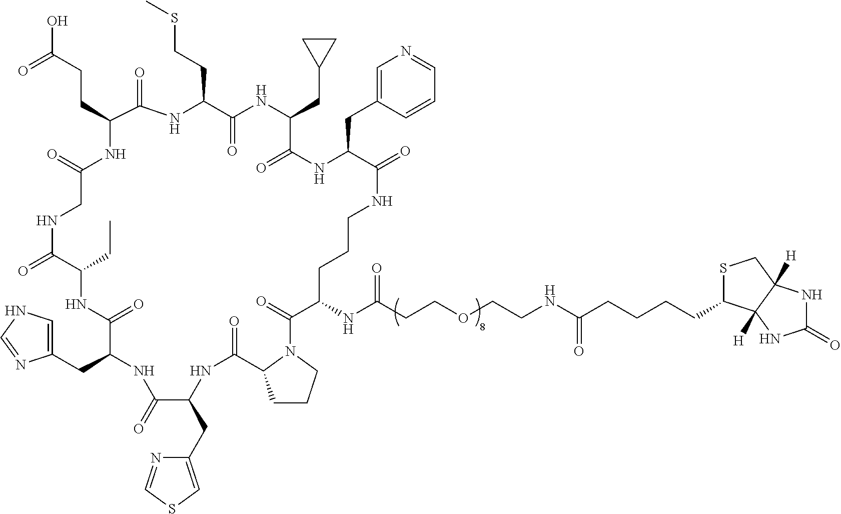 | I-9 |
| 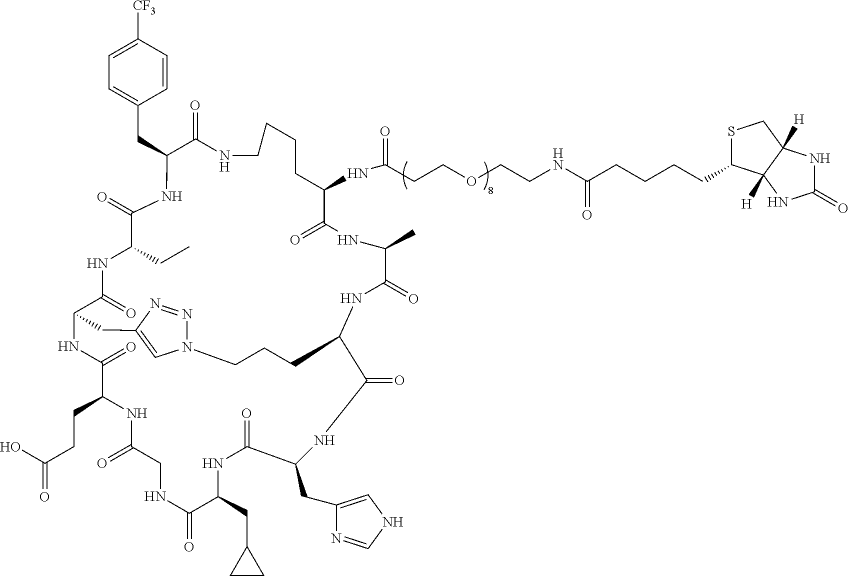 | I-10 |
| 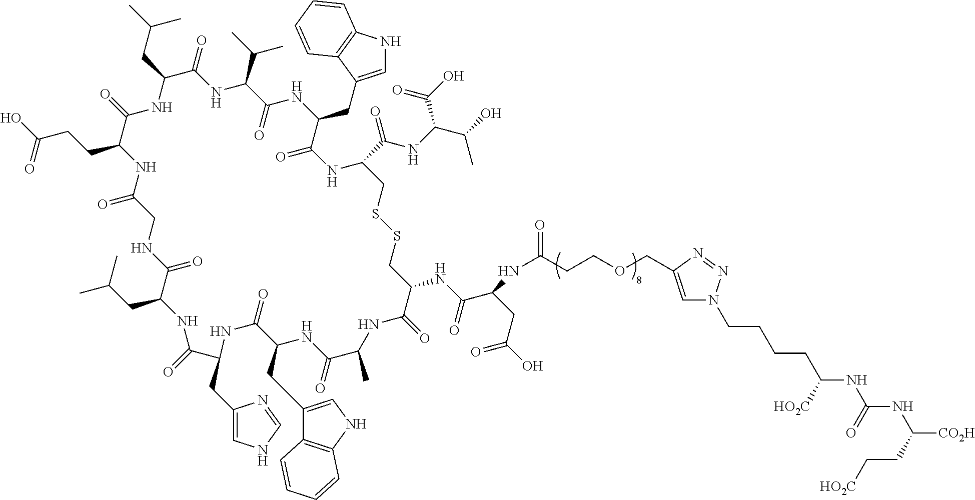 | I-11 |
| 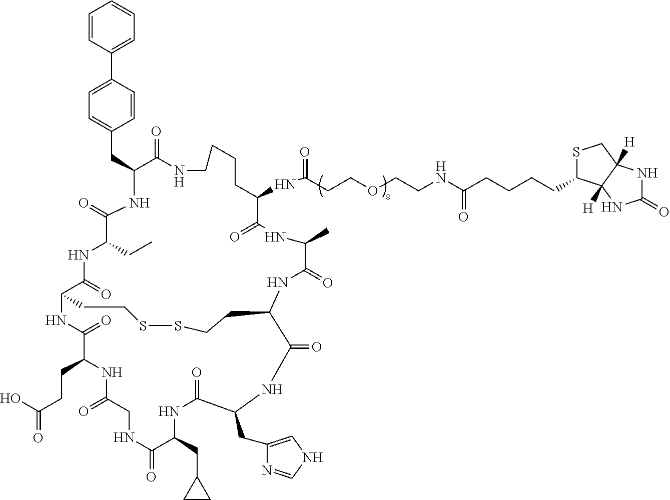 | I-12 |
| 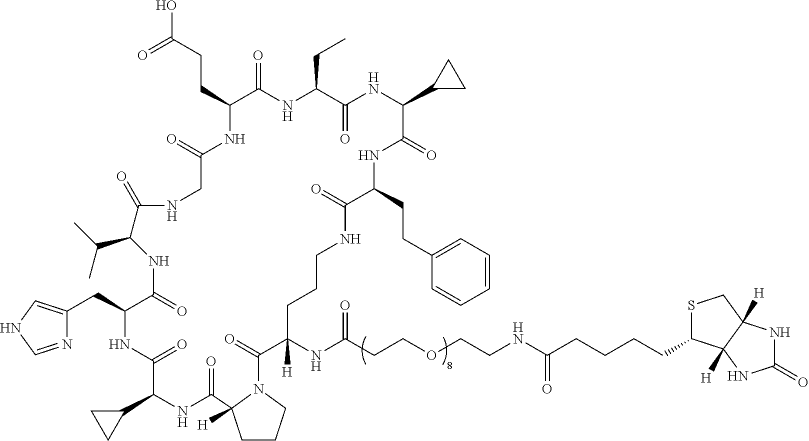 | I-13 |
| 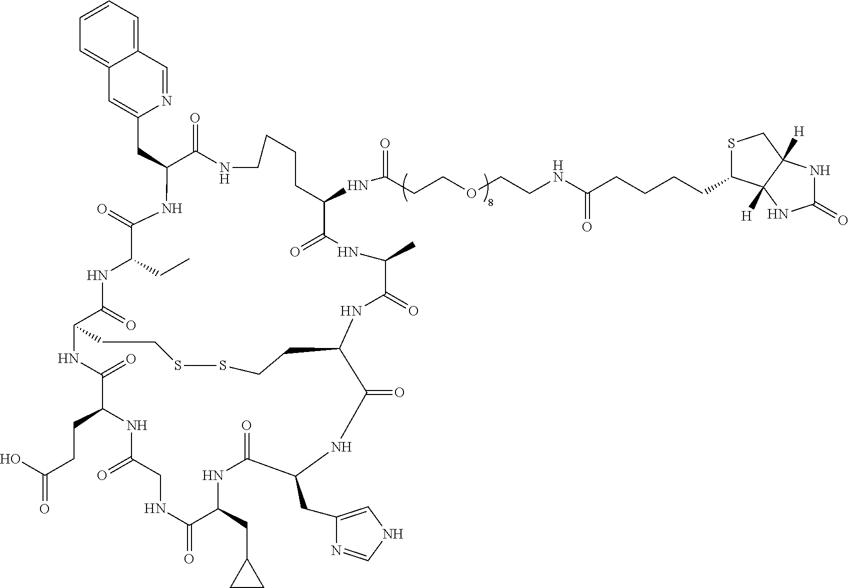 | I-14 |
| 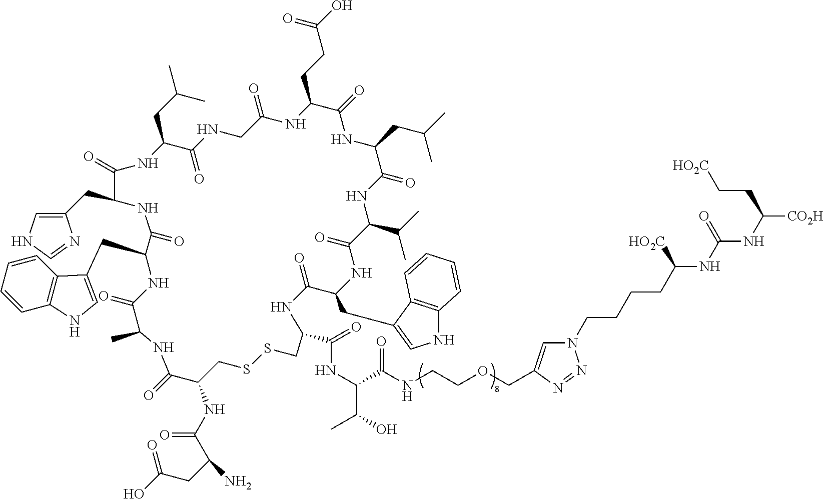 | I-15 |
| 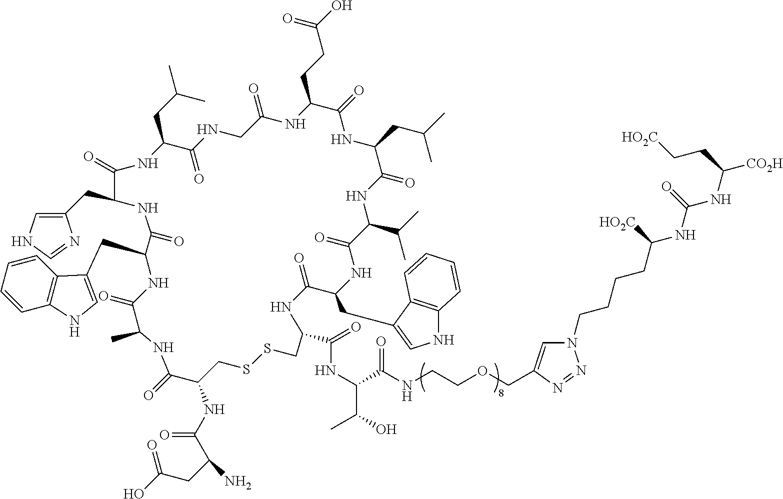 | I-16 |
| 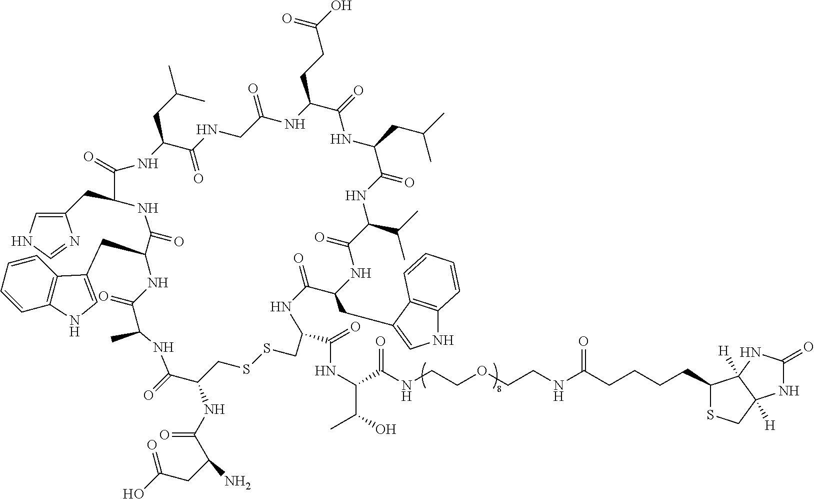 | I-17 |
| 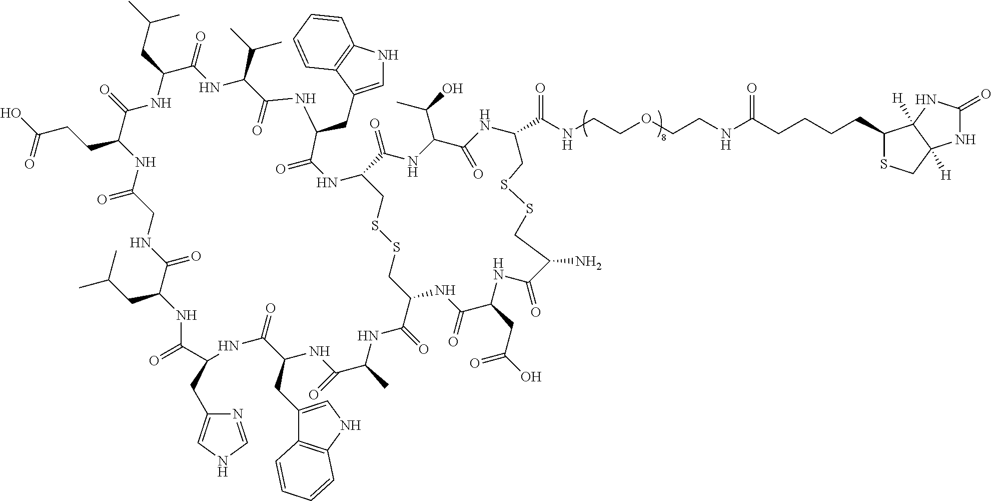 | I-18 |
| 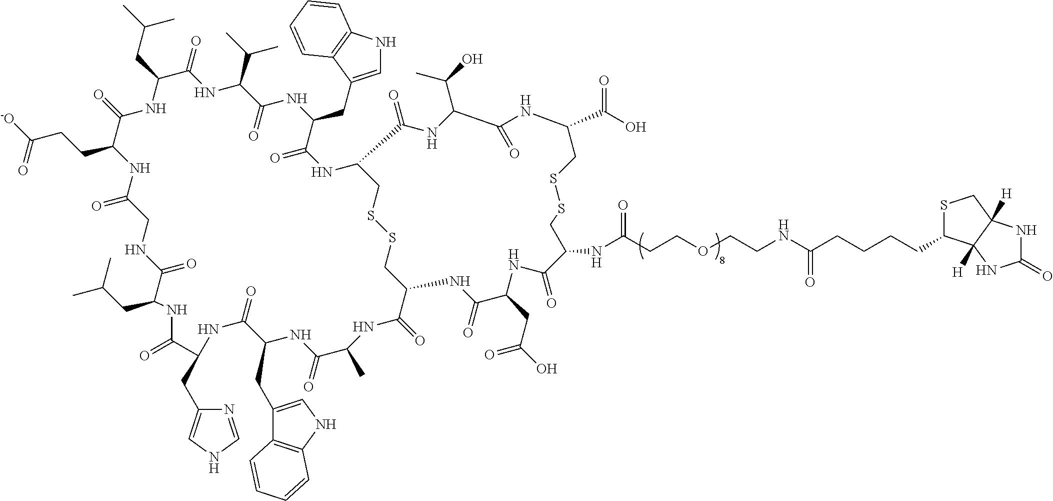 | I-19 |
| 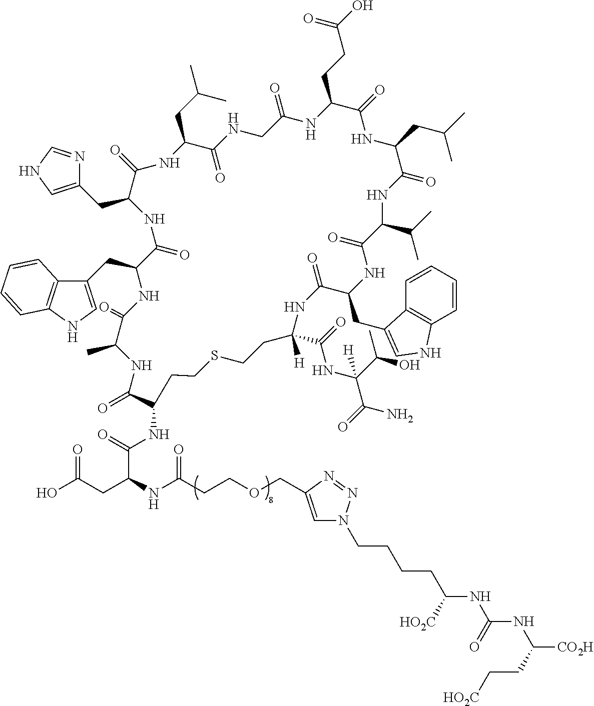 | I-20 |
| 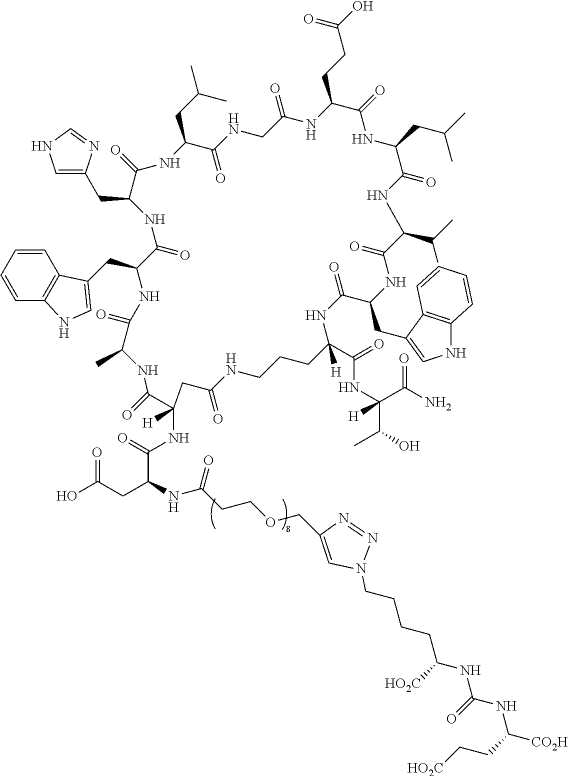 | I-21 |
| 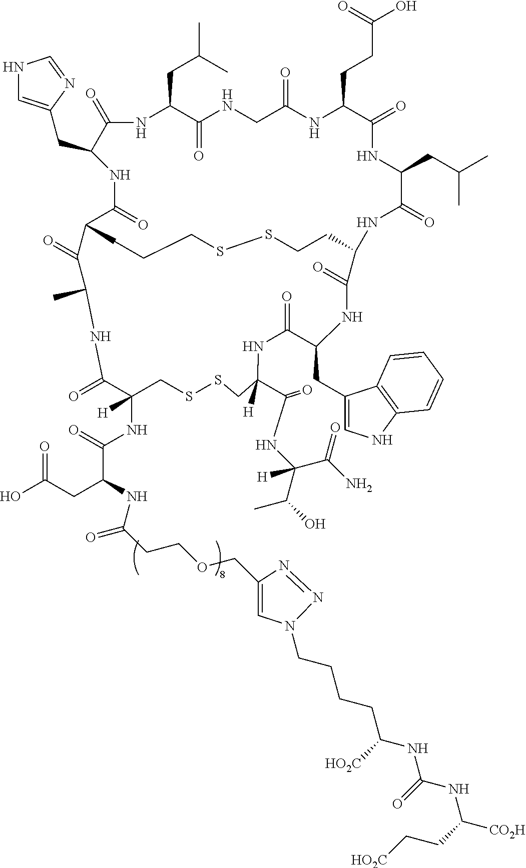 | I-22 |
| 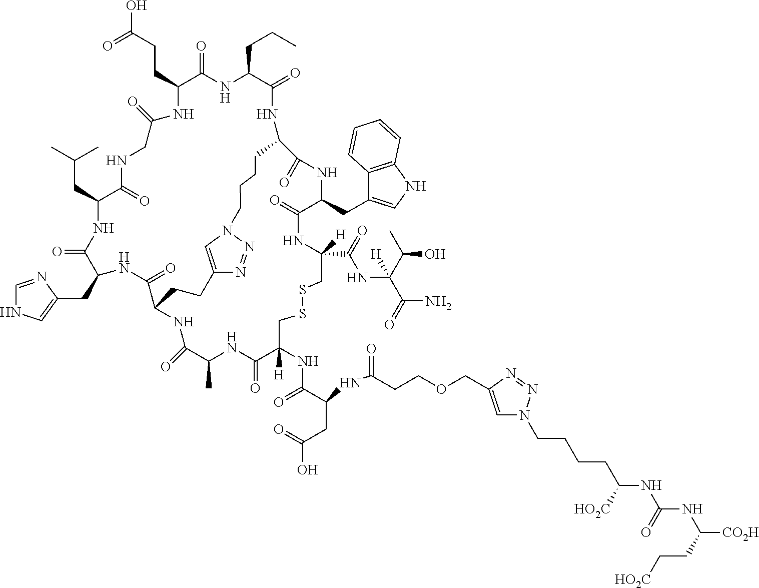 [SMILES: CCC[C@@H]1NC(=O)[C@H](CCC(=O)O)NC(=O)CNC(=O)[C@H](CC(C)C)NC(=O)[C@H](Cc2c[nH]cn2)NC(=O)[C@@H]2CCc3cn(nn3)CCCC[C@H](NC1=O)C(=O)N[C@@H](Cc1c[nH]c3ccccc13)C(=O)N[C@H](C(=O)N[C@H](C(N)=O)[C@@H](C)O)CSSC[C@H](NC(=O)[C@H](CC(=O)O)NC(=O)CCOCc1cn(CCCC[C@H](NC(=O)N[C@@H](CCC(=O)O)C(=O)O)C(=O)O)nn1)C(=O)N[C@@H](C)C(=O)N2] | I-23 |
| 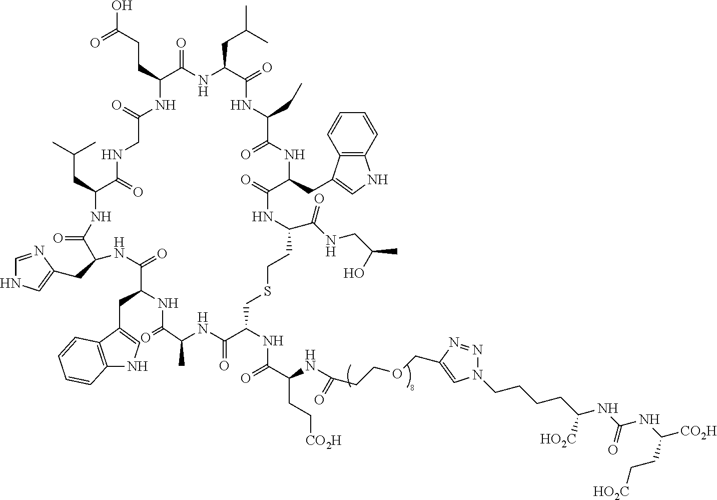 | I-24 |
| 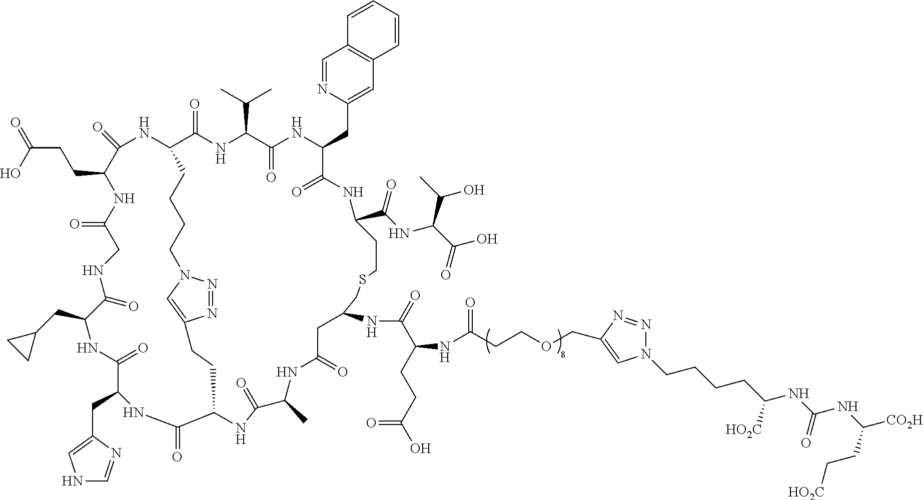 | I-25 |
| 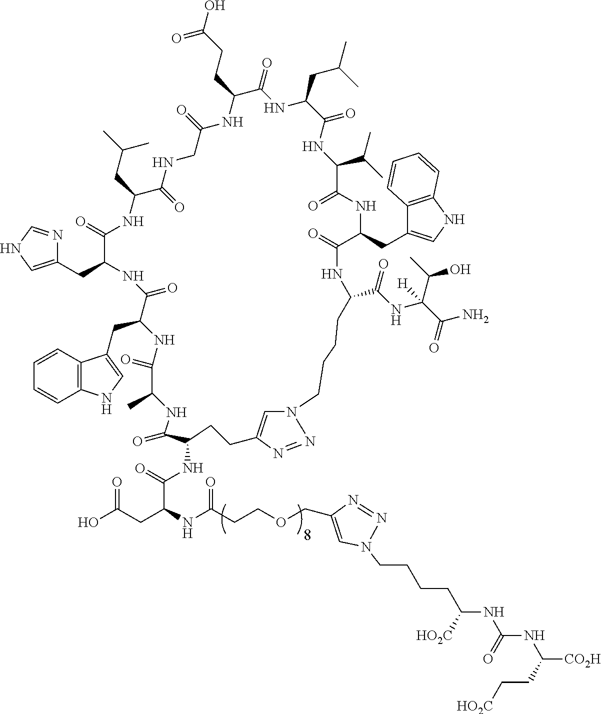 | I-26 |
| 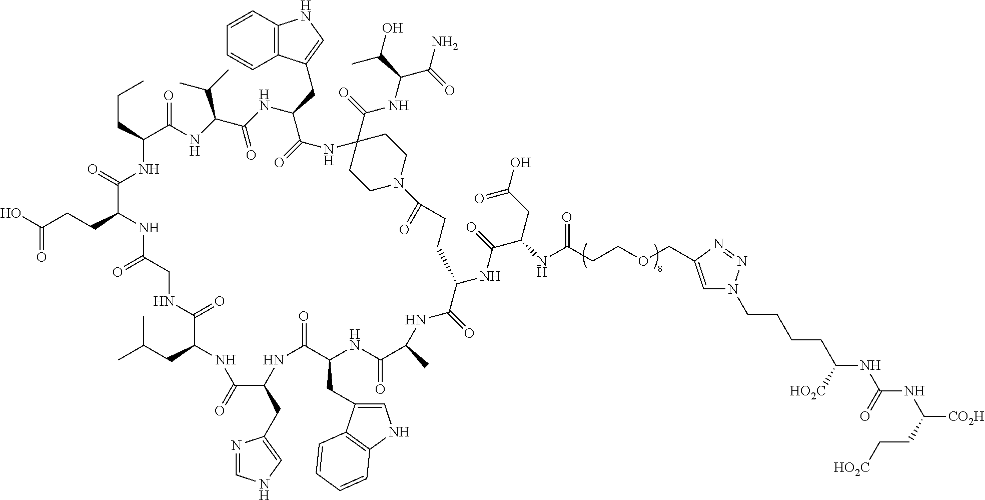 | I-27 |
| 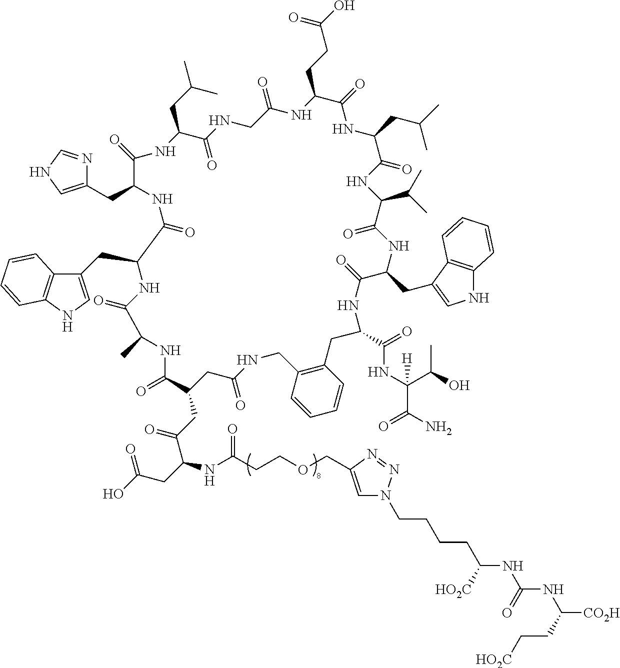 | I-28 |
| 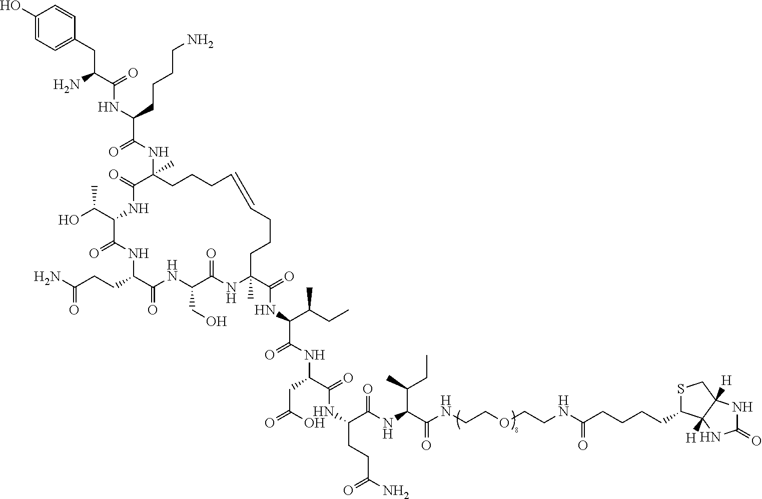 | I-29 |
| 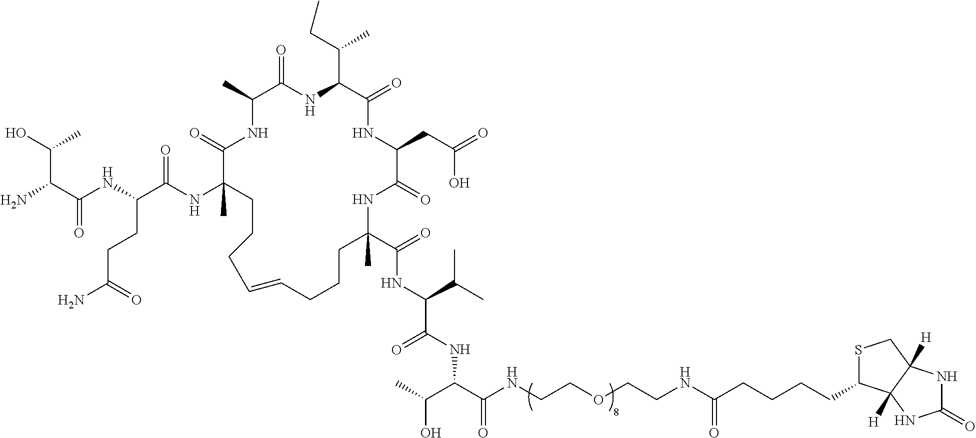 | I-30 |
| 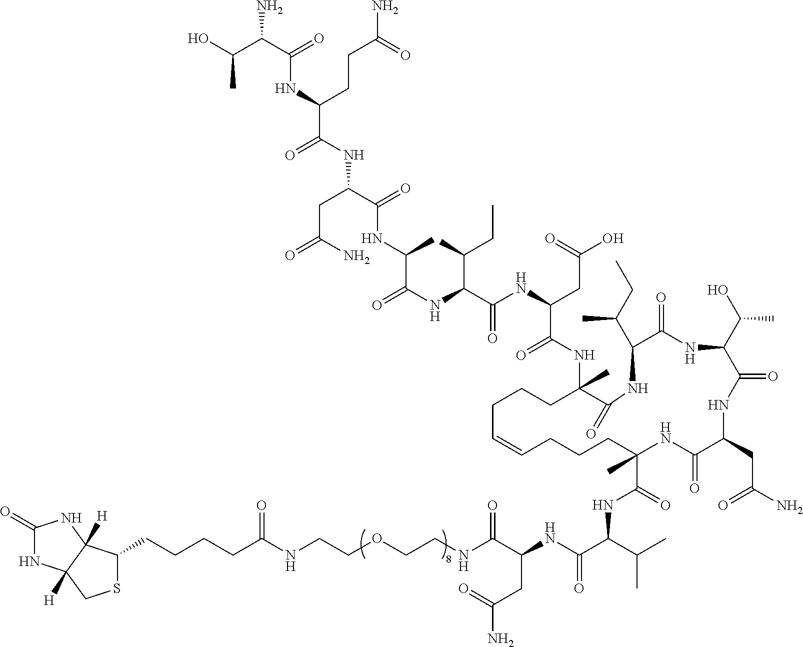 | I-31 |
| 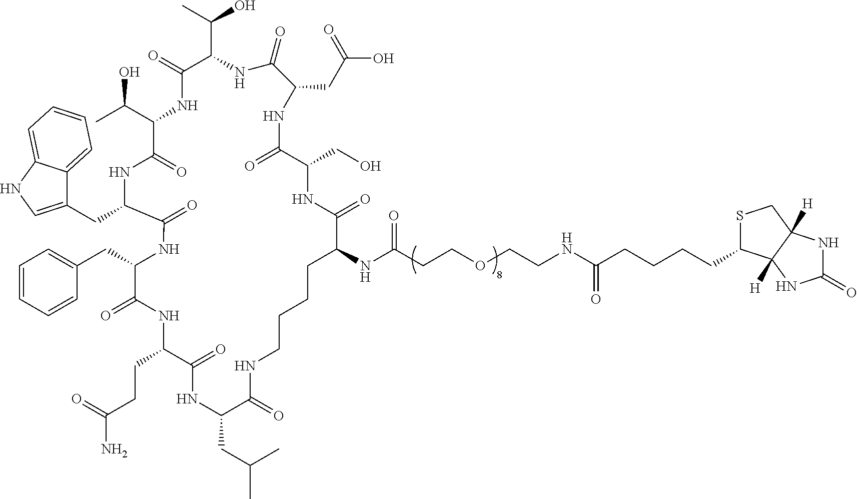 | I-32 |
| 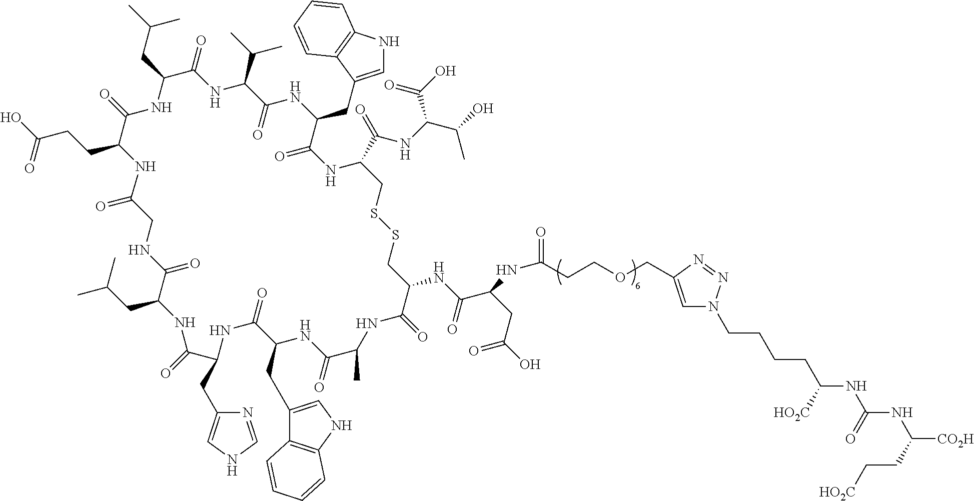 | I-33 |
| 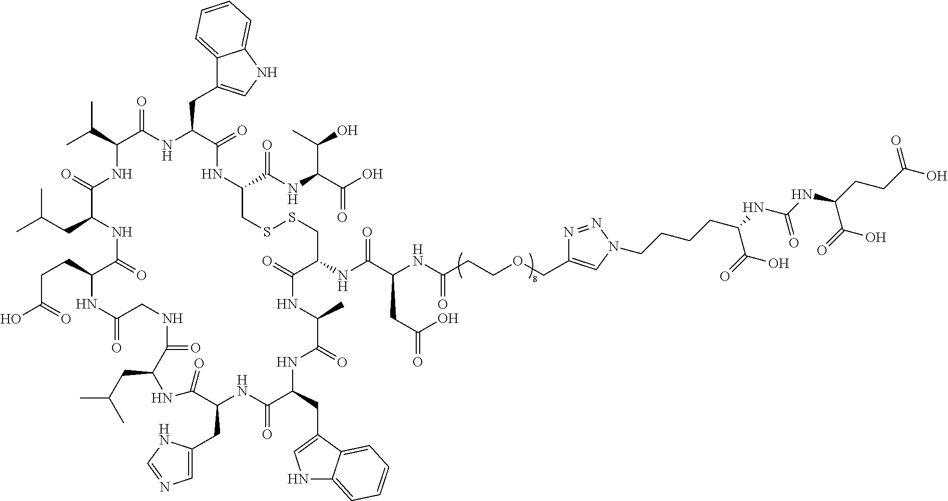 | I-34 |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3rd edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio) pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl) ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that compounds of formula I, II or III may contain one or more stereocenters, and may be present as an racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5th Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

In some embodiments, the present disclosure provides compounds that are useful for preparing ARMs. In some embodiments, the present disclosure provides compounds that are useful for construction of ARM molecules through cycloaddition reactions, e.g., click chemistry or variants thereof.

In some embodiments, the present disclosure provides a compound having the structure of formula IV:

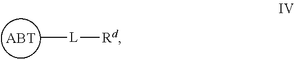

or a salt thereof, wherein
ABT is an antibody binding moiety;
L is a linker moiety;
R^{d} is -L^{a}-R′, wherein R^{d} comprises —C≡C— or —N₃;
each L^{a} is independently a covalent bond, or an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the present disclosure provides a compound of formula IV-a:

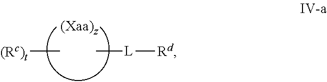

or a salt thereof, wherein each variable is independently as described in the present disclosure.

In some embodiments, the present disclosure provides a compound of formula IV-b:

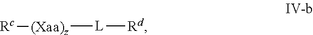

or a salt thereof, wherein each variable is independently as described in the present disclosure.

In some embodiments, the present disclosure provides a compound of formula IV-c:

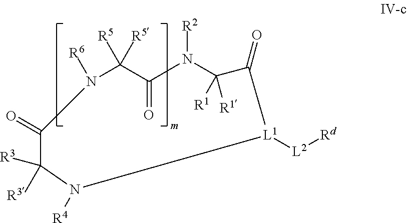

or a salt thereof, wherein each variable is independently as described in the present disclosure.

In some embodiments, the present disclosure provides a compound of formula IV-d:

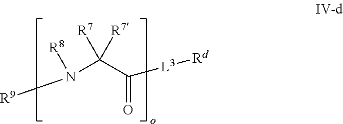

or a salt thereof, wherein each variable is independently as described in the present disclosure.

In some embodiments, the present disclosure provides a compound of formula V:

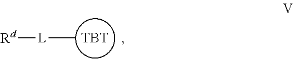

or a salt thereof, wherein each variable is independently as described in the present disclosure.

In some embodiments, the present disclosure provides a method for preparing a compound, comprising steps of:
providing a first compound of formula IV, IV-a, IV-b, IV-c, or IV-d, or a salt thereof, wherein the compound comprising a first reactive moiety;
providing a second compound of formula V comprising a second reactive moiety or a salt thereof; and
reacting the first compound with the second compound, wherein the first reactive moiety reacts with the second reaction moiety through a cycloaddition reaction.

Many cycloaddition reactions can be utilized in accordance with the present disclosure. In some embodiments, a cycloaddition reaction is a [4+2] reaction. In some embodiments, a cycloaddition reaction is a [3+2] reaction. In some embodiments, a [3+2] reaction is a click chemistry reaction. In some embodiments, a first reactive moiety is —C≡C— and the second reactive moiety is —N₃. In some embodiments, a first reactive moiety is —N₃ and the second reactive moiety is —C≡C—.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that it is effective to redirect endogenous antibodies selectively to diseased cells, e.g., cancer cells, thereby inducing antibody-directed, cell-mediated immunity, e.g., cytotoxicity. In certain embodiments, the amount of compound in compositions of this invention is such that is effective to redirect endogenous antibodies selectively to cancer cells, thereby inducing antibody-directed, cell-mediated cytotoxicity, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful to redirect endogenous antibodies selectively to diseased cells, e.g., cancer cells, thereby inducing an antibody-directed, cell-mediated immune response, e.g., cytotoxicity.

In some embodiments, the present disclosure provides methods for recruiting antibodies, e.g., endogenous antibodies, to a target comprising contacting the target with a provided agent, compound or composition. In some embodiments, recruited antibodies comprise one or more endogenous antibodies. In some embodiments, recruited antibodies have specificity toward one or more antigens. In some embodiments, recruited antibodies have specificity toward one or more peptide antigens or proteins. In some embodiments, recruited antibodies are heterogeneous in that they are not antibodies toward the same antigen or protein.

In some embodiments, the present disclosure provides methods for recruiting an immune cell to a target, comprising contacting a target with a provided agent, compound or composition.

In some embodiments, the present disclosure provides methods for triggering, generating, encouraging, and/or enhancing one or more immune system activities toward a target, comprising contacting a target with a provided agent, compound or composition. In some embodiments, an immune system activity is or comprises ADCC. In some embodiments, an immune system activity is or comprises ADCP. In some embodiments, an immune system activity is or comprises both ADCC and ADCP. In some embodiments, an immune system activity is or comprises complement dependent cytotoxicity (CDC). In some embodiments, an immune system activity is or comprises ADCVI.

In some embodiments, a target is a cancer cell. In some embodiments, a target is a cancer cells in a subject. In some embodiments, provided methods comprise administering a provided agent, compound or composition to a subject.

In some embodiments, when contacted with its target, provided agents and compounds form complexes with antibodies and Fc receptors on target cells. In some embodiments, the present disclosure provides a complex comprising:
an agent comprising:
   an antibody binding moiety,
   a target binding moiety, and
   optionally a linker moiety,
an Fc region, and
an Fc receptor,
   wherein the antibody binding moiety is a universal antibody binding moiety.

In some embodiments, the present disclosure provides a complexes comprising two or more complexes each independently comprising:
an agent comprising:
   an antibody binding moiety,
   a target binding moiety, and
   optionally a linker moiety,
an Fc region, and
an Fc receptor,
   wherein Fc regions of the complexes are of antibodies and/or fragments thereof toward different antigens or proteins.

In some embodiments, Fc regions of the complexes are of antibodies and/or fragments thereof toward different proteins. In some embodiments, one or more Fc regions are of endogenous antibodies and/or fragments thereof.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In some embodiments, the present invention provides a method for treating one or more disorders, diseases, and/or conditions wherein the disorder, disease, or condition is a cancer.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Representative cancers include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. Among other things, provided technologies (e.g., compounds, compositions, methods, etc.) are particularly useful for preventing and/or treating cancer.

Furthermore, the invention provides the use of a compound according to the definitions herein, or a pharmaceutically acceptable salt, or a hydrate or solvate thereof for the preparation of a medicament for the treatment of a proliferative disease.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of formula I, II or III and one or more additional therapeutic agents. The therapeutic agent may be administered together with a compound of formula I, II or III, or may be administered prior to or following administration of a compound of formula I, II or III. Suitable therapeutic agents are described in further detail below. In certain embodiments, a compound of formula I, II or III may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I, II or III may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a compound of formula I, II or III and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a compound of formula I, II or III and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a compound of formula I, II or III and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a compound of formula I, II or III and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a compound of formula I, II or III and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a compound of formula I, II or III and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a compound of formula I, II or III and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a compound of formula I, II or III and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present invention provides a method of treating HIV comprising administering to a patient in need thereof a compound of formula I, II or III and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a compound of formula I, II or III and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a compound of formula I, II or III and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a compound of formula I, II or III and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a compound of formula I, II or III and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a compound of formula I, II or III and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a compound of formula I, II or III and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, the present invention provides a method of treating Alzheimer's disease comprising administering to a patient in need thereof a compound of formula I, II or III and one or more additional therapeutic agents selected from donepezil (Aricept®), rivastigmine (Excelon®), galantamine (Razadyne®), tacrine (Cognex®), and memantine (Namenda®).

In another embodiment, the present invention provides a method of treating organ transplant rejection or graft vs. host disease comprising administering to a patient in need thereof a compound of formula I, II or III and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a compound of formula I, II or III and a BTK inhibitor, wherein the disease is selected from inflammatory bowel disease, arthritis, systemic lupus erythematosus (SLE), vasculitis, idiopathic thrombocytopenia purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenia purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenia states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenia purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a compound of formula I, II or III and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a compound of formula I, II or III and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

The compounds and compositions, according to the method of the present invention, may be administered using any amount and any route of administration effective for treating or lessening the severity of a cancer or a proliferative disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

Among other things, compounds and/or compositions of the present disclosure can be employed in combination therapies, that is, compounds and/or compositions of the present disclosure can be administered concurrently with, prior to, or subsequent to, one or more other therapeutic agents or medical procedures, particularly for treatment of various cancers. In some embodiments, a compound of the current invention may also be used to advantage in combination with other antiproliferative compounds. The particular combination of therapies (therapeutics or procedures) to employ in a combination regimen will take into account compatibility of a desired other therapeutics and/or procedures and the desired therapeutic effect to be achieved. It will also be appreciated that the therapies employed may achieve a desired effect for the same disorder (for example, a provided compound may be administered concurrently with another anticancer agent), or they may achieve different effects (e.g., control of any adverse effects). In some embodiments, a therapeutic agent is a chemotherapeutic agent or antiproliferative compounds. Exemplary chemotherapy agents include but are not limited to alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, other immunotherapeutic drugs, and other anticancer agents. Further, provided technologies can be used together with hypoleukocytosis (neutrophil) medicines that are cancer treatment adjuvant, thrombopenia medicines, antiemetic drugs, and cancer pain medicines for patient's QOL recovery or be made as a mixture with them. In some embodiments, a therapeutic reagent is an antibody. In some embodiments, a therapeutic agent is an immunomodulatory agent. In some embodiments, an immunomodulatory agent targets cell surface signaling molecules on immune cells. In some embodiments, an immunomodulatory agent targets cell surface signaling molecules on immune cells, wherein the agent is an antagonist blocking a co-inhibitory pathway. In some embodiments, an immunomodulatory agent is a checkpoint blockage agent. In some embodiments, an immunomodulatory agent is an antibody targeting a cell surface signaling protein expressed by immune cells. In some embodiments, an immunomodulatory agent is an antibody targeting a protein selected from PD-1, PD-L1, CTLA4, TIGIT, BTLA, TIM-3, LAG3, B7-H3, and B7-H4. In some embodiments, an immunomodulatory agent is a PD-1 antibody (e.g., nivolumab, pembrolizumab, pidilizumab, BMS 936559, MPDL3280A, etc). In some embodiments, an immunomodulatory agent is a PD-L1 antibody. In some embodiments, an immunomodulatory agent is a CTLA4 antibody (e.g., ipilimumab). In some embodiments, an immunomodulatory agent is a TIGIT antibody. In some embodiments, an immunomodulatory agent is a BTLA antibody. In some embodiments, an immunomodulatory agent is a Tim-3 antibody. In some embodiments, an immunomodulatory agent is a LAG3 antibody. In some embodiments, an immunomodulatory agent is a B7-H3 antibody. In some embodiments, an immunomodulatory agent is a B7-H4 antibody. In some embodiments, an immunomodulatory agent targets cell surface signaling molecules on immune cells, wherein the agent is an agonist engaging a co-stimulatory pathway. In some embodiments, such an immunomodulatory agent is or comprises an antibody targeting a co-stimulatory receptor. In some embodiments, an antibody activates a T cell co-stimulatory receptor. In some embodiments, an antibody targets a member of the tumor necrosis factor (TNF) receptor superfamily. In some embodiments, an antibody targets a protein selected from CD137 (4-1BB), CD357 (GITR, TNFRS18, AITR), CD134 (OX40) and CD 40 (TNFRSF5). In some embodiments, an antibody is an anti-CD137 antibody (e.g., urelumab). In some embodiments, an antibody is an anti-CD357 antibody. In some embodiments, an antibody is an anti-CD40 antibody. In some embodiments, an antibody is an anti-CD134 antibody. Additional exemplary T cell co-stimulatory and co-inhibitory receptors are described in Chen L, Flies D B., Molecular mechanisms of T cell co-stimulation and co-inhibition. Nat. Rev. Immunol. 2013, 13 (4), 227-42, and Yao S, et al., Advances in targeting cell surface signalling molecules for immune modulation. Nat. Rev. Drug Discov. 2013, 12 (2), 136-40. In some embodiments, a therapeutic agent is an antibodies activating such a stimulatory receptor, or blocking such an inhibitory receptor.

In some embodiments, one or more other therapeutic agents are or comprise tumor-specific immune cells. In some embodiments, one or more other therapeutic agents are or comprise tumor-specific T cells. In some embodiments, one or more other therapeutic agents are or comprise tumor-infiltrating lymphocytes (TILs). In some embodiments, one or more other therapeutic agents are or comprise T cells ectopically expressing a known anti-tumor T cell receptor (TCR). In some embodiments, one or more other therapeutic agents are or comprise chimeric antigen receptors (CAR) T cells. In some embodiments, a provided composition comprises an immunopotentiative substance. Exemplary immunopotentiative substances that can be used in combination with provided compounds, compositions and/or methods include but are not limited to various cytokines and tumor antigens. Cytokines that stimulate immune reactions include, for example, GM-CSF, M-CSF, G-CSF, interferon-α, β, γ, IL-1, IL-2, IL-3, and IL-12, etc. Antibodies to block inhibitory receptors and/or to activate stimulatory receptors, which are widely known in the art and described herein, for example but not limited to, B7 ligand derivatives, anti-CD3 antibodies, anti-CD28 antibodies, and anti-CTLA-4 antibodies can also improve the immune reactions. In some embodiments, a therapeutic agent is a small molecule for immune modulation. In some embodiments, a therapeutic agent is a small molecule that mediating anti-tumor immune activity. In some embodiments, a therapeutic agent is a small molecule that targets an enzyme directly involved in immune regulation. In some embodiments, a therapeutic agent is an indoleamine 2,3-dioxygenase (IDO) inhibitor. In some embodiments, a therapeutic agent is an IDO1 inhibitor, e.g., F001287, indoximod, NLG-919 and INCB024360. In some embodiments, a therapeutic agent is a tryptophan-2,3 dioxygenase (TDO) inhibitor. In some embodiments, a therapeutic agent is an IDO/TDO dual inhibitor. In some embodiments, a therapeutic agent is an IDO-selective inhibitor. In some embodiments, some other embodiments, a therapeutic agent is a TDO-selective inhibitor. In some embodiments, a provided composition comprises an IDO inhibitor and a first construct. In some embodiments, a provided composition comprises an IDO inhibitor, a first construct and a second construct. It is recognized that immune response to a first construct and/or a second construct can be significantly enhanced by administration of an IDO inhibitor.

In some embodiments, a medical procedure that may be used in combination with compounds, compositions and methods of the present application include but are not limited to surgery, radiotherapy (□-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF), to name a few), hyperthermia, cryotherapy, and adoptive T-cell transfer (e.g., TIL therapy, transgenic TCRs, CAR T-cell therapy, NK cellular therapy, etc.). In some embodiments, a medical procedure is surgery. In some embodiments, a medical procedure is radiotherapy.

Antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; Ilmofosine; RO 318220 and RO 320432; GO 6976; Isis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C₁₋₁₀₃₃, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3 Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™ Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4th Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives. Also included are in particular those compounds, proteins or monoclonal antibodies of VEGF such as 1-(4-chloroanilino)-4-(4-pyridylmethyl) phthalazine or a pharmaceutically acceptable salt thereof, 1-(4-chloroanilino)-4-(4-pyridylmethyl) phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR¹⁰, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a compound of this invention. Implantable devices coated with a compound of this invention are another embodiment of the present invention.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1. Synthesis of Exemplary Compounds

Peptides were prepared using standard, automated Fluoroenylmethyloxycarbonyl (Fmoc) solid phase peptide synthesis procedures.

### Peptide Synthesis:

The peptide was synthesized using standard Fmoc chemistry.
1) Add DCM to the vessel containing CTC Resin (0.1 mmol) and Fmoc-Trp (Boc)-OH (42.5 mg, 0.08 mmol, 0.80 eq) with N₂ bubbling.
2) Add DIEA (4.0 eq) dropwise and mix for 2 hours.
3) Add MeOH (0.1 mL) and mix for 30 min.
4) Drain and wash with DMF for 5 times.
5) Add 20% piperidine/DMF and react on 30 min.
6) Drain and wash with DMF for 5 times.
7) Add Fmoc-amino acid solution and mix 30 seconds, then add activation buffer, N2 bubbling for about 1 hour.
8) Repeat step 4 to 7 for next amino acid coupling.

Note: Biotin-PEG8—CH₂CH₂COOH (1.5 eq) was coupled using HATU (1.5 eq) and DIEA (3.0 eq). The other amino acids were coupled by 3 equivalent using HBTU (2.85 eq) and DIEA (6 eq). 20% piperidine in DMF was used for Fmoc deprotection for 30 min. The coupling reaction was monitored by ninhydrin test, and the resin was washed with DMF for 5 times.

### Peptide Cleavage and Purification:

1) Add cleavage buffer (95% TFA/2.5% TIS/2.5% H₂O) to the flask containing the side chain protected peptide at room temperature and stir for 3 hours.
2) The peptide is precipitated with cold Isopropyl ether and centrifuged (3 min at 3000 rpm).
3) Isopropyl ether washes two additional times.
4) Dry the crude peptide under vacuum 2 hours.
5) Purify the crude peptide by Prep-HPLC (A: 0.1% TFA in H₂O, B: ACN) and lyophilized to give the final product (64.3 mg, 52.3% yield).

### Purification Conditions:

Reverse phase HPLC (Gilson 281) was carried out on Luna C18 (200×25 mm; 10 um) and Gemini C18 (150*30 mm; 5 μm) in series. Solvent A: water with 0.075% trifluoroacetic acid; Solvent B: acetonitrile. Gradient: at room temperature, 15% of B to 45% of B within 60 min at 20 mL/min; then 90% B at 20 mL/min over 10 min, UV detection (wave length=215 nm). The peptide was lyophilized to give the desired product (64.3 mg, 52.3% yield) as a white solid.

All final compounds showed the correct mass for the desired compound.

### Example 2. Synthesis of I-13

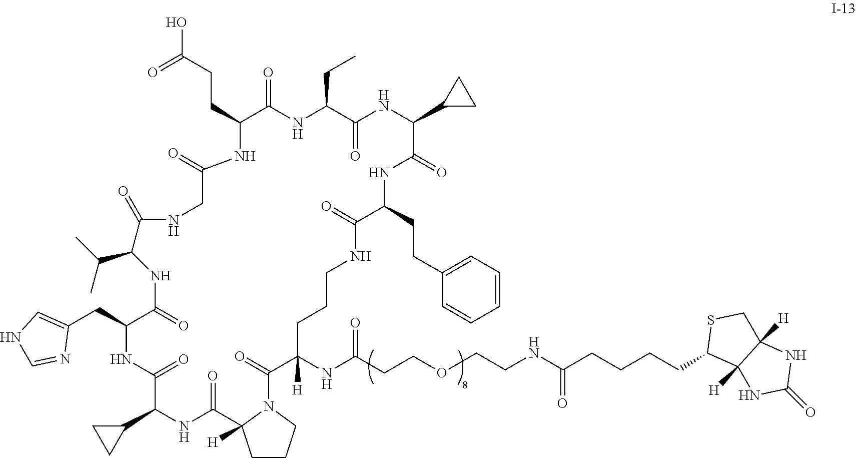

### Peptide Synthesis:

The peptide was synthesized using standard Fmoc chemistry.
1) Add DCM to the vessel containing CTC Resin (0.4 mmol) and Fmoc-Gly-OH (356.7 mg, 1.2 mmol, 3.0 eq) with N₂ bubbling.
2) Add DIEA (6.0 eq) dropwise and mix for 2 hours.
3) Add MeOH (2 mL) and mix for 30 min.
4) Drain and wash with DMF for 5 times
5) Add 20% piperidine/DMF and react for 30 min.
6) Drain and wash with DMF for 5 times
7) Add Fmoc-amino acid solution and mix 30 seconds, then add activation buffer, N₂ bubbling for about 1 hour.
8) Repeat steps 4 to 7 for next amino acid coupling.
9) Dde protecting group was deprotected by 3% hydrazine in DMF for 2 times with each of 20 min.

### Note:

Synthesized scale: 0.4 mmol.

TABLE 2
Fmoc amino acids utilized in the Synthesis of I-13.

| # | Materials | Coupling reagents |
|---|---|---|
| 1 | Fmoc-Gly-OH (3 eq) | DIEA (6 eq) |
| 2 | Fmoc-Abu-OH (2 eq) | HBTU (1.95 eq) and DIEA (4 eq) |
| 3 | Fmoc-His(Trt)-OH (2 eq) | HBTU (1.95 eq) and DIEA (4 eq) |
| 4 | (S)-2-((((9H-fluoren-9- | HBTU (1.95 eq) and DIEA (4 eq) |
| | yl)methoxy)carbonyl)amino)-2-(thiazol- | |
| | 4-y1)aceticacid (2 eq) | |
| 5 | Fmoc-D-Pro-OH (2 eq) | HBTU (1.95 eq) and DIEA (4 eq) |
| 6 | Fmoc-Orn(Dde)-OH (2 eq) | HBTU (1.95 eq) and DIEA (4 eq) |
| 7 | Fmoc-NH-PEG8-CH₂CH₂COOH (1.5 | HBTU (1.45 eq) and DIEA (4 eq) |
| | eq) | |
| 8 | D-Biotin (2 eq) | HBTU (1.95 eq) and DIEA (4 eq) |
| 9 | (S)-2-((((9H-fluoren-9- | HBTU (1.95 eq) and DIEA (4 eq) |
| | yl)methoxy)carbonyl)amino)-4- | |
| | phenylbutanoic acid (2 eq) | |
| 10 | Fmoc-Cyclopropylalanine (2 eq) | HBTU (1.95 eq) and DIEA (4 eq) |
| 11 | (S)-2-((((9H-fluoren-9- | HBTU (1.95 eq) and DIEA (4 eq) |
| | yl)methoxy)carbonyl)amino)butanoic | |
| | acid (2 eq) | |
| 12 | Fmoc-Glu(OtBu)-OH (2 eq) | HBTU (1.95 eq) and DIEA (4 eq) |

20% piperidine in DMF was used for Fmoc deprotection for 30 min. The coupling reaction was monitored by ninhydrin test, and the resin was washed with DMF for 5 times.

### Peptide Cleavage, Cyclizatoin and Purification:

6) Add cleavage buffer (1% TFA/99% DCM 14 mL) to the flask containing the side chain protected peptide at room temperature and stir for 0.5 hours.
7) The reaction mixture was filtered and the resulting filtrate was diluted by dry DCM to 1 mM. DIEA was added to adjust the pH to about 8. To above solution was added TBTU (835.30 mg, 1.2 mmol, 3.0 eq) and HOBT (162.15 mg, 1.2 mmol, 3.0 eq) and the resulting solution was reacted for 3-4 h at room temperature. The reaction was monitored by LCMS.
8) After reacted completely, the reaction mixture was washed with 1 N HCl (200 mL) for one time. And the organic phase was dried under vacuum to obtain the crude peptide, which was treated with the cocktail of 95% TFA/2.5% H₂O/2.5% TIPS for about 1 h.
9) The crude peptide was precipitated out by methyl tert-butyl ether.
10) Purify the crude peptide by HPLC (A: H₂O, B: ACN) to give the final product (78.90 mg, 11.44% yield).

### Purification Conditions:

Reverse phase HPLC (Gilson 281) was carried out on Luna C18 (200×25 mm; 10 um) and Gemini C18 (150*30 mm; 5 μm) in series. Solvent A: water with 0.1% trifluoroacetic acid; Solvent B: acetonitrile with 0.1% trifluoroacetic acid. Gradient: at room temperature, 5% of B to 35% of B within 60 min at 20 mL/min; then 90% B at 20 mL/min over 10 min, UV detection (wave length=215 nm). The peptide was lyophilized to give the desired product, I-13 (78.90 mg, 11.44%) as a white solid.

### Example 3. Synthesis of I-29

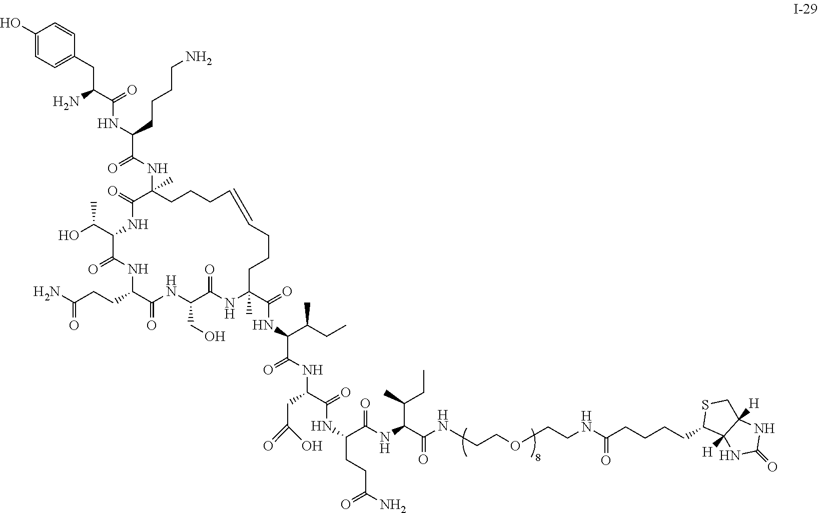

### General Procedure for Preparation of Compound 3.1

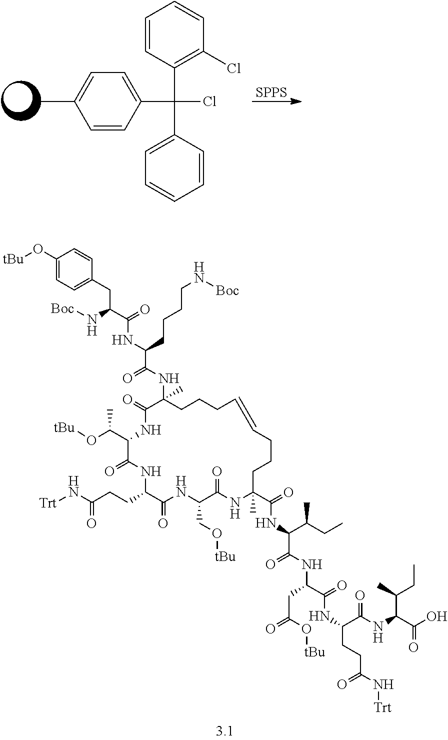

### Peptide Synthesis:

The peptide was synthesized using standard Fmoc chemistry.

Synthesis scale: 0.2 mmol.
1) Add DCM to the vessel containing CTC Resin and Fmoc-Ile-OH (70.68 mg, 200.00 μmol, 1 eq) with N₂ bubbling.
2) Drain and then DMF wash 30 sec with 3 times.
3) Add 20% piperidine/DMF and mix for 30 min.
4) Drain and then DMF wash 30 sec with 5 times.
5) Add Fmoc-amino acid solution and mix 30 seconds, then add coupling reagents with N₂ bubbling for about 1 hour.
6) Repeat step 2 to 5 for next amino acid coupling.
7) When the coupling was finished, the resin in DCE was added Grubb's 1^{st} (20%) with N₂ bubbling for 16 hours at 25° C.

TABLE 3
Fmoc Amino Acids used in the Preparation of I-29.

| # | Materials | Coupling reagents |
|---|---|---|
| 1 | Fmoc-Ile-OH (1 eq) | DIEA (4 eq) |
| 2 | Fmoc-Gln(Trt)-OH (3 eq) | HBTU (2.9 eq) and DIEA (6 eq) |
| 3 | Fmoc-Asp(OtBu)-OH (3 eq) | HBTU (2.9 eq) and DIEA (6 eq) |
| 4 | Fmoc-Ile-OH(3 eq) | HBTU (2.9 eq) and DIEA (6 eq) |
| 5 | S5 (1.5 eq) | HATU (1.5 eq) and DIEA (3 eq) |
| 6 | Fmoc-Ser(tBu)-OH (3 eq) | HBTU (2.9 eq) and DIEA (6 eq) |
| 7 | Fmoc-Gln(Trt)-OH (3 eq) | HBTU (2.9 eq) and DIEA (6 eq) |
| 8 | Fmoc-Thr(tBu)-OH (3 eq) | HBTU (2.9 eq) and DIEA (6 eq) |
| 9 | S5 (1.5 eq) | HBTU (1.5 eq) and DIEA (3 eq) |
| 10 | Fmoc-Lys(Boc)-OH (3 eq) | HBTU (2.9 eq) and DIEA (6 eq) |
| 11 | Fmoc-Tyr(tBu)-OH (3 eq) | HBTU (2.9 eq) and DIEA (6 eq) |
| 12 | (Boc)₂O (3 eq) | DIEA (6 eq) |

20% piperidine in DMF was used for Fmoc deprotection for 30 min. The coupling reaction was monitored by ninhydrin test, and the resin was washed with DMF for 5 times.

Peptide Cleavage and Work up:
1) Add cleavage buffer (1% TFA/99% DCM) to the flask containing the side chain protected peptide at room temperature and stir for 0.5 hour and filtrated.
2) The filtration is added DIEA to neutralized and extracted with water twice.
3) Dry the crude peptide under vacuum 16 hours to give crude compound as brown solid (290 mg, 64% yield).

### General Procedure for Preparation of Compound 3.3

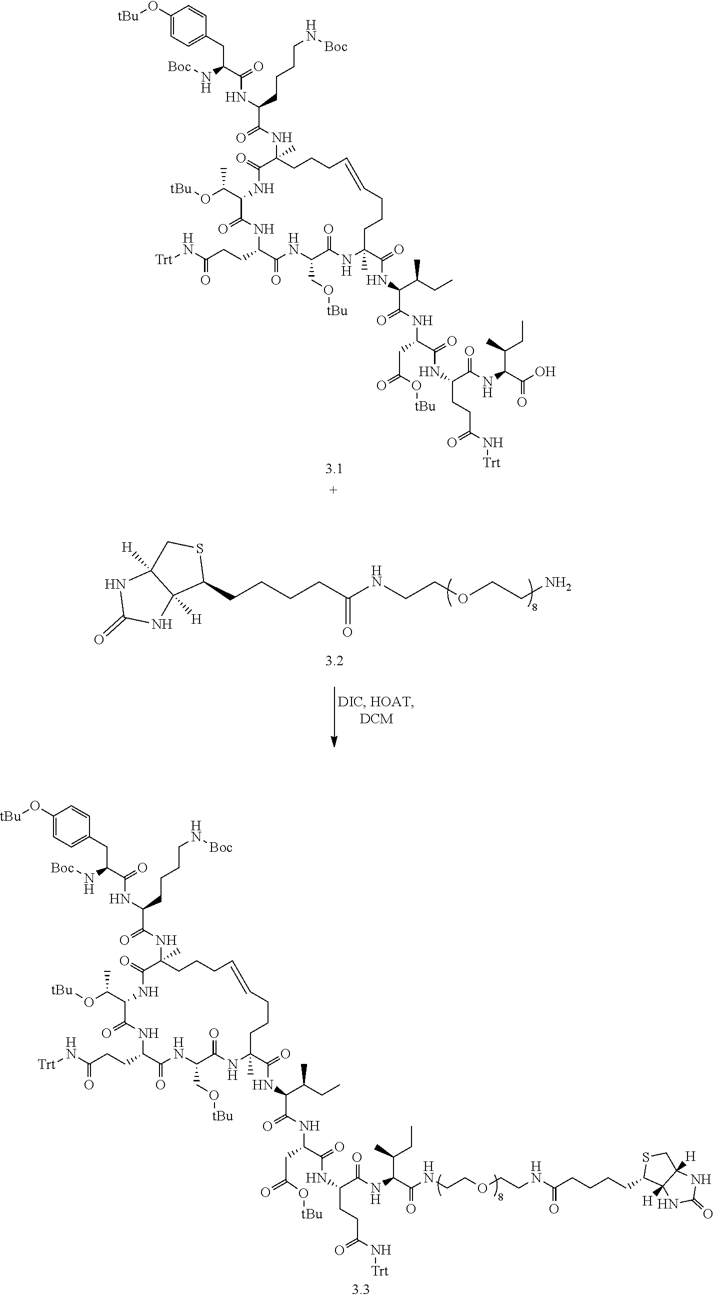

To a mixture of compound 3.1 (290 mg, 128.61 μmol, 1 eq), compound 3.2 (164.32 mg, 257.23 μmol, 2 eq) and HOAt (35.01 mg, 257.23 μmol, 2 eq) in DCM (10 mL) was added DIC (32.46 mg, 257.23 μmol, 39.83 μL, 2 eq) in one portion at 25° C. under N₂. The mixture was stirred at 25° C. for 5 hours. LC-MS showed compound 3.1 was consumed completely. The reaction mixture was extracted with 1M HCL 30 mL (10 mL*3) and dried over Na₂SO₄, filtered and concentrated under reduced pressure to give a crude compound 3.3 (300 mg, 81% yield).

### General Procedure for Preparation of I-29

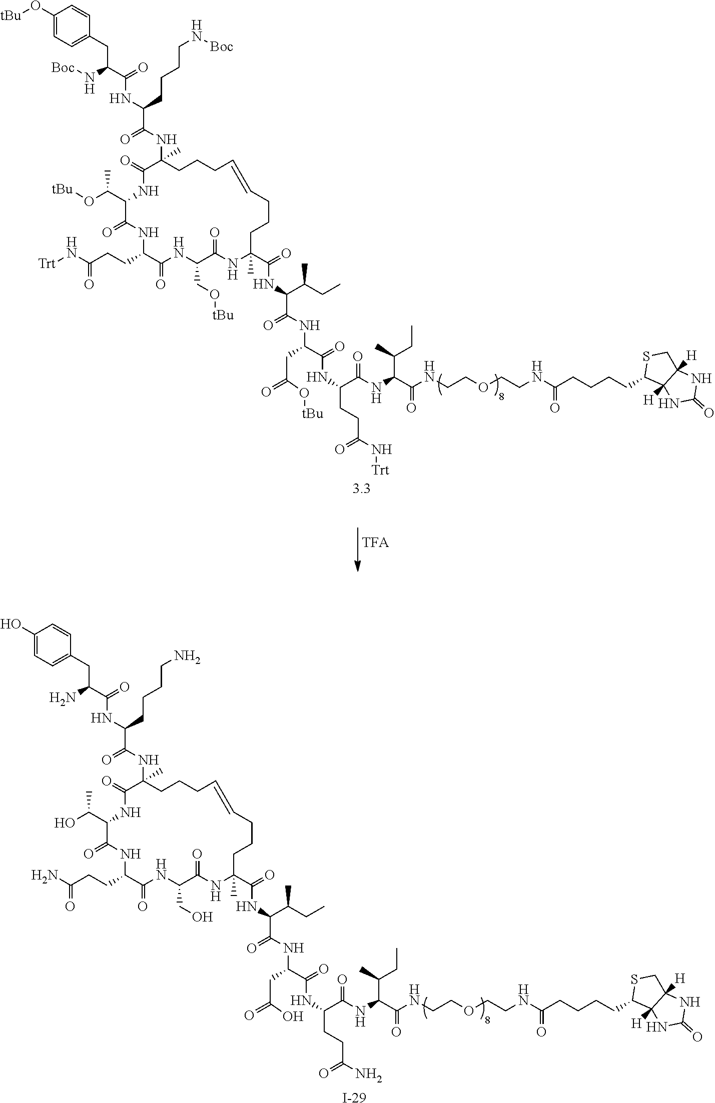

To a mixture of Compound 3.3 (300 mg, 113.93 μmol, 1 eq) was added 95% TFA/2.5% TIS/2.5% H₂O in one portion at 25° C. The mixture was stirred at 25° C. for 2 hours. LC-MS showed Compound 3.3 was consumed completely. The peptide is precipitated with cold tert-butyl methyl ether and centrifuged (2 min at 3000 rpm). The residue was purified by prep-HPLC (0.075% TFA/H₂O, ACN) to give I-29 (75.5 mg, 38.40 μmol, 33.70% yield) as a white solid.

### Example 4. Synthesis of I-30

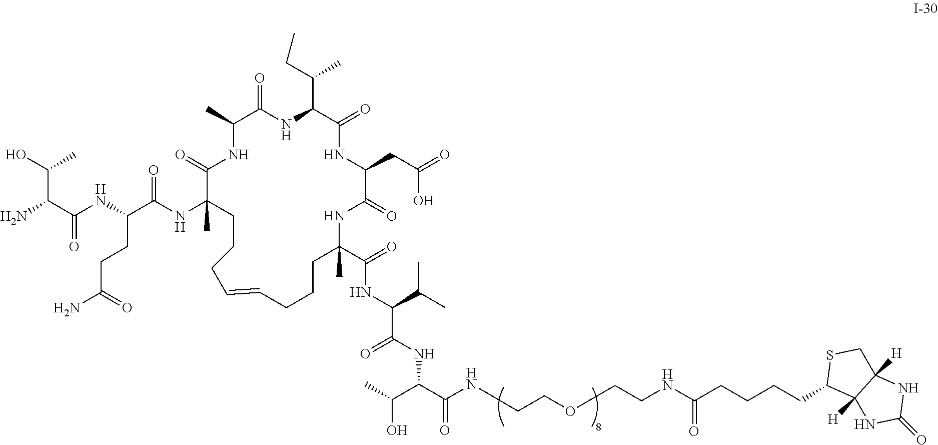

### General Procedure for Preparation of Compound 4.1

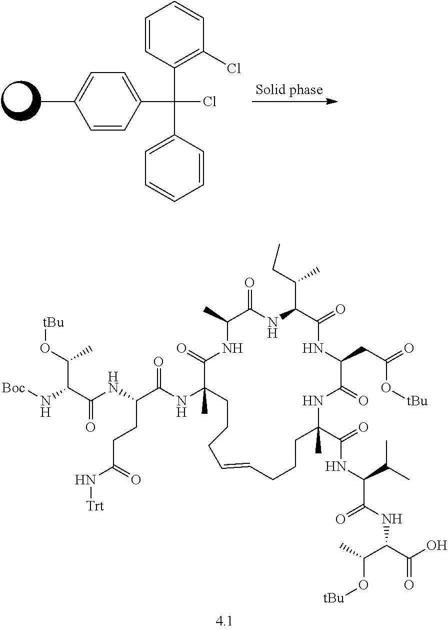

### Peptide Synthesis:

The peptide was synthesized using standard Fmoc chemistry.

Synthesis scale: 0.2 mmol.
1) Add DCM to the vessel containing CTC Resin and Fmoc-Thr(tBu)-OH (79.68 mg, 200.00 umol, 1 eq) with N₂ bubbling.
2) Drain and then DMF wash 30 sec with 3 times.
3) Add 20% piperidine/DMF and mix for 30 min.
4) Drain and then DMF wash 30 sec with 5 times.
5) Add Fmoc-amino acid solution and mix 30 seconds, then add coupling reagents with N₂ bubbling for about 1 hour.
6) Repeat step 2 to 5 for next amino acid coupling.
7) When the coupling was finished, the resin in DCE was added Grubb's 1^{st} (20%) with N₂ bubbling for 16 hours at 25° C.

TABLE 4
Fmoc Amino Acids used in the synthesis of I-30.

| # | Materials | Coupling reagents |
|---|---|---|
| 1 | Fmoc-Thr(tBu)-OH (1 eq) | DIEA (4 eq) |
| 2 | Fmoc-Val-OH (3 eq) | HBTU (2.9 eq) and DIEA (6 eq) |
| 3 | S5 (1.5 eq) | HATU (1.5 eq) and DIEA (3 eq) |
| 4 | Fmoc-Asp(OtBu)-OH (3 eq) | HBTU (2.9 eq) and DIEA (6 eq) |
| 5 | Fmoc-Ile-OH (3 eq) | HBTU (2.9 eq) and DIEA (6 eq) |
| 6 | Fmoc-Ala-OH (3 eq) | HBTU (2.9 eq) and DIEA (6 eq) |
| 7 | S5 (1.5 eq) | HATU (1.5 eq) and DIEA (3 eq) |
| 8 | Fmoc-Gln(Trt)-OH (3 eq) | HBTU (2.9 eq) and DIEA (6 eq) |
| 9 | Fmoc-Thr(tBu)-OH (3 eq) | HBTU (2.9 eq) and DIEA (6 eq) |
| 10 | (Boc)₂O (3 eq) | DIEA (6 eq) |

20% piperidine in DMF was used for Fmoc deprotection for 30 min. The coupling reaction was monitored by ninhydrin test, and the resin was washed with DMF for 5 times.

### Peptide Cleavage and Work Up:

1) Add cleavage buffer (1% TFA/99% DCM) to the flask containing the side chain protected peptide at room temperature and stir for 0.5 hour and filtrated.
2) The filtration is added DIEA to neutralized and extracted with water twice.
3) Dry the crude peptide under vacuum 16 hours to give crude compound as brown solid (110 mg, 36% yield).

### General Procedure for Preparation of Compound 4.2

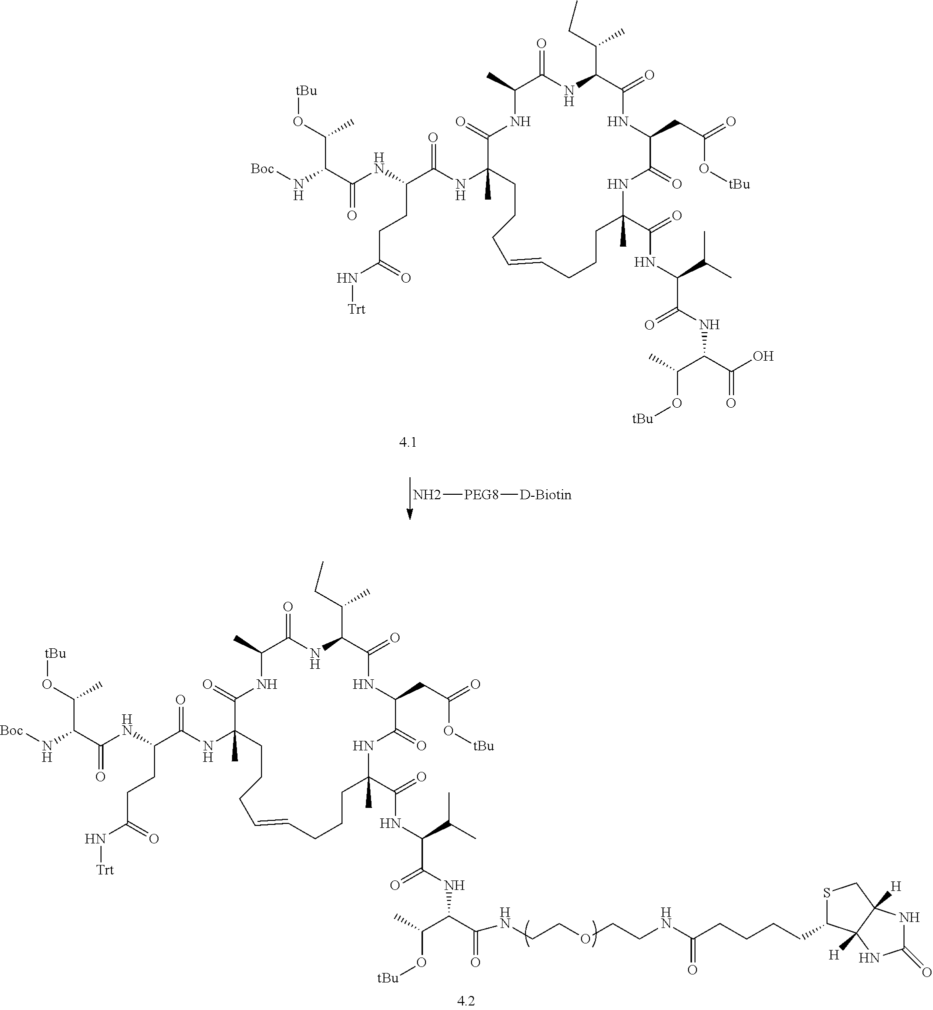

To a mixture of compound 4.1 (110 mg, 72.9 μmol, 1 eq), NH₂-PEG8-D-Biotin (93.2 mg, 145.9 μmol, 2 eq) and HOAt (19.86 mg, 145.9 μmol, 2 eq) in DCM (10 mL) was added DIC (18.41 mg, 145.9 μmol, 22.59 μL, 2 eq) in one portion at 25° C. under N₂. The mixture was stirred at 25° C. for 5 hours. LC-MS showed compound 4.1 was consumed completely. The reaction mixture was extracted with 1M HCL 30 mL (10 mL*3) and dried over Na₂SO₄, filtered and concentrated under reduced pressure to give a crude compound 4.2 (100 mg, 64.4% yield).

### General Procedure for Preparation of I-30

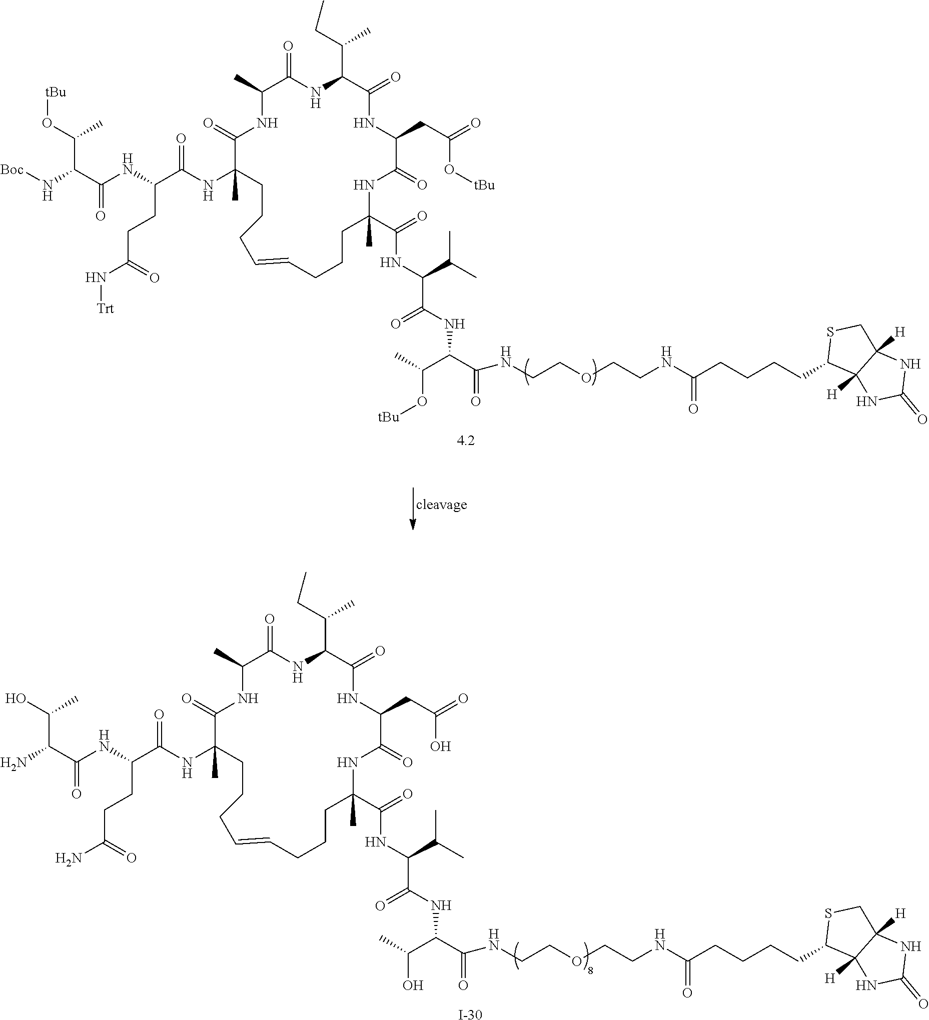

To a mixture of Compound 4.2 (100 mg, 47 μmol, 1 eq) was added 95% TFA/TIS/EDT/H2O in one portion at 25° C. The mixture was stirred at 25° C. for 2 hours. LC-MS showed Compound 3 was consumed completely. The peptide is precipitated with cold tert-butyl methyl ether and centrifuged (2 min at 5000 rpm). The residue was purified by prep-HPLC (0.075% TFA/H2O, ACN) to give I-30 (24.2 mg, 15.00 μmol, 32% yield) as a white solid.

### Example 5. Synthesis of I-31

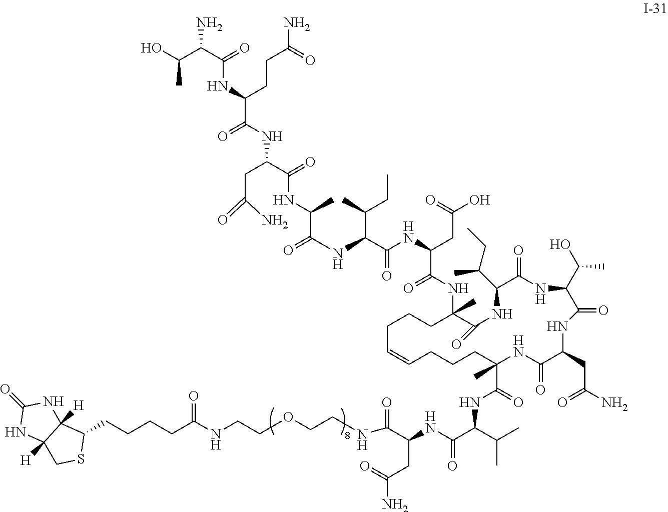

### General Procedure for Preparation of Compound 5.1

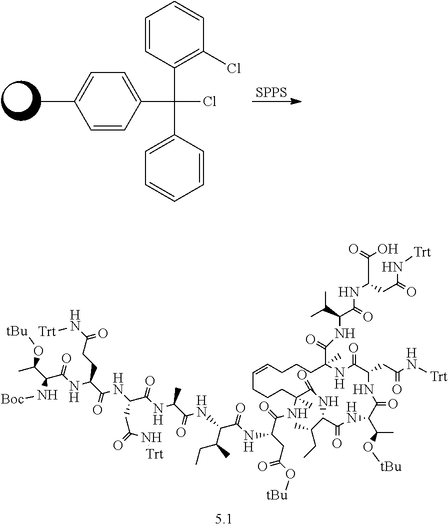

### Peptide Synthesis:

The peptide was synthesized using standard Fmoc chemistry.

Synthesis scale: 0.2 mmol.
1) Add DCM to the vessel containing CTC Resin and Fmoc-Asn(Trt)-OH (119.2 mg, 200.00 umol, 1 eq) with N₂ bubbling.
2) Drain and then DMF wash 30 sec with 3 times.
3) Add 20% piperidine/DMF and mix for 30 min.
4) Drain and then DMF wash 30 sec with 5 times.
5) Add Fmoc-amino acid solution and mix 30 seconds, then add coupling reagents with N₂ bubbling for about 1 hour.
6) Repeat step 2 to 5 for next amino acid coupling.
7) When the coupling was finished, the resin in DCE was added Grubb's 1^{st} (20%) with N₂ bubbling for 16 hours at 25° C.

TABLE 5
Fmoc Amino Acids used in the Synthesis of I-31.

| # | Materials | Coupling reagents |
|---|---|---|
| 1 | Fmoc-Asn(Trt)-OH(1 eq) | DIEA (4 eq) |
| 2 | Fmoc-Val-OH (3 eq) | HBTU (2.9 eq) and DIEA (6 eq) |
| 3 | S5 (1.5 eq) | HATU (1.5 eq) and DIEA (3 eq) |
| 4 | Fmoc-Asn(Trt)-OH (3 eq) | HBTU (2.9 eq) and DIEA (6 eq) |
| 5 | Fmoc-Thr(tBu)-OH (3 eq) | HBTU (2.9 eq) and DIEA (6 eq) |
| 6 | Fmoc-Ile-OH (3 eq) | HBTU (2.9 eq) and DIEA (6 eq) |
| 7 | S5 (1.5 eq) | HATU (1.5 eq) and DIEA (3 eq) |
| 8 | Fmoc-Asp(OtBu)-OH (3 eq) | HBTU (2.9 eq) and DIEA (6 eq) |
| 9 | Fmoc-Ile-OH (3 eq) | HBTU (2.9 eq) and DIEA (6 eq) |
| 10 | Fmoc-Ala-OH (3 eq) | HBTU (2.9 eq) and DIEA (6 eq) |
| 11 | Fmoc-Asn(Trt)-OH (3 eq) | HBTU (2.9 eq) and DIEA (6 eq) |
| 12 | Fmoc-Gln(Trt)-OH (3 eq) | HBTU (2.9 eq) and DIEA (6 eq) |
| 13 | Fmoc-Thr(tBu)-OH (3 eq) | HBTU (2.9 eq) and DIEA (6 eq) |
| 14 | (Boc)₂O (3 eq) | DIEA (6 eq) |

20% piperidine in DMF was used for Fmoc deprotection for 30 min. The coupling reaction was monitored by ninhydrin test, and the resin was washed with DMF for 5 times.

### Peptide Cleavage and Work Up:

1) Add cleavage buffer (1% TFA/99% DCM) to the flask containing the side chain protected peptide at room temperature and stir for 0.5 hour and filtrated.
2) The filtration is added DIEA to neutralized and extracted with water twice.
3) Dry the crude peptide under vacuum 16 hours to give crude compound as brown solid (335 mg, 62.29% yield).

### General Procedure for Preparation of Compound 5.3

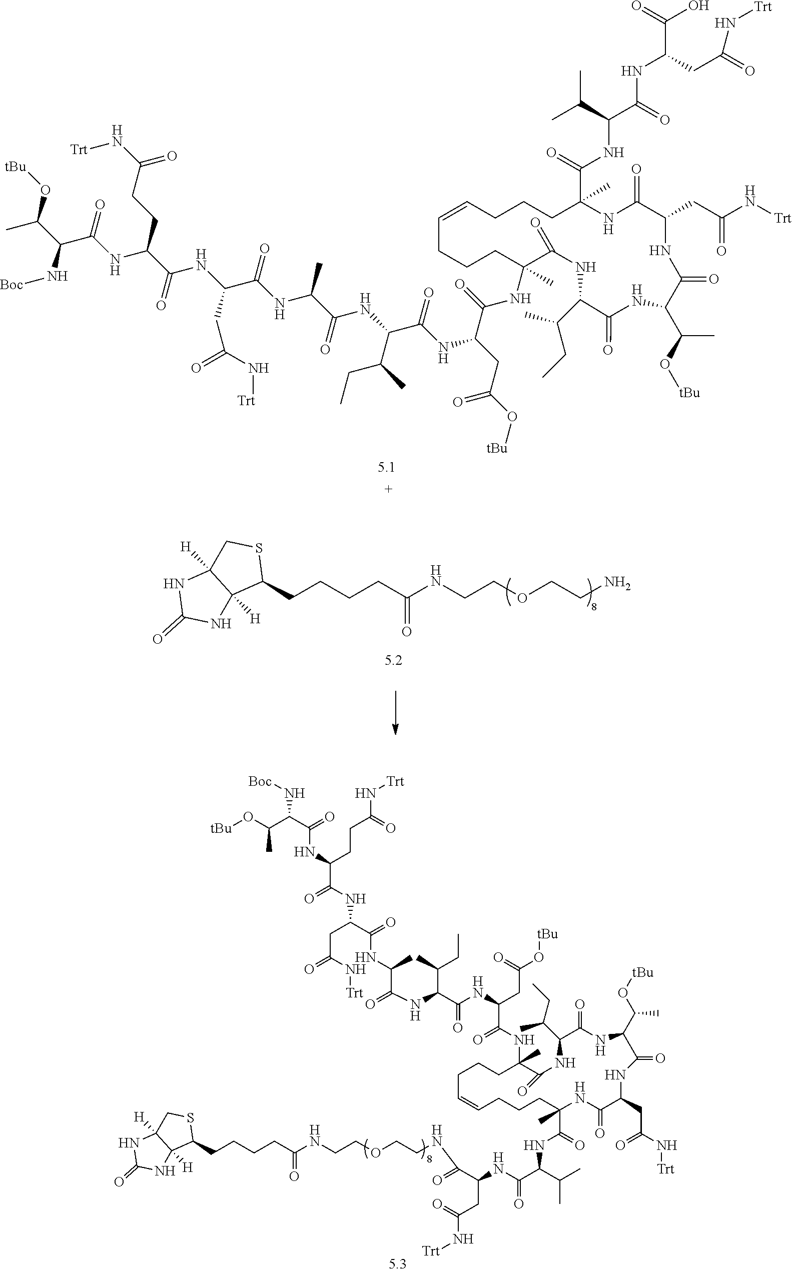

To a mixture of compound 5.1 (335 mg, 124.52 μmol, 1 eq), compound 5.2 (159.09 mg, 249.04 μmol, 2 eq) and HOAt (33.9 mg, 249.04 μmol, 2 eq) in DCM (10 mL) was added DIC (249.04 μmol, 38.59 μL, 2 eq) in one portion at 25° C. under N₂. The mixture was stirred at 25° C. for 5 hours. LC-MS showed compound 5.1 was consumed completely. The reaction mixture was extracted with 1M HCL 30 mL (10 mL*3). Dried over Na₂SO₄, filtered and concentrated under reduced pressure to give a crude compound 5.3 (350 mg, 84.89% yield).

### General Procedure for Preparation of I-31

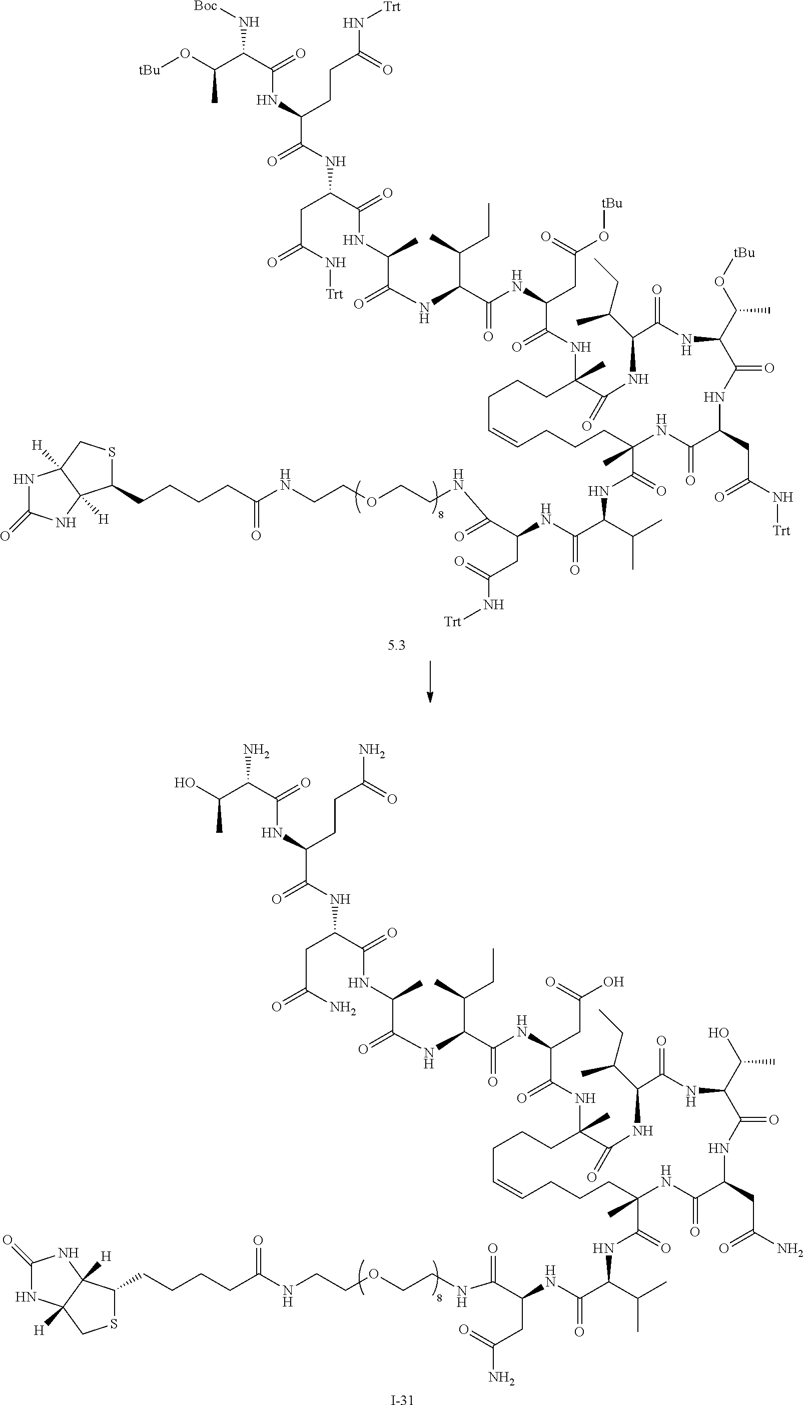

To a mixture of Compound 5.3 (350 mg, 105.71 μmol, 1 eq) was added 95% TFA/TIS/EDT/H2O in one portion at 25° C. The mixture was stirred at 25° C. for 2 hours. LC-MS showed Compound 5.3 was consumed completely. The peptide is precipitated with cold tert-butyl methyl ether and centrifuged (2 min at 5000 rpm). The residue was purified by prep-HPLC (0.075% TFA/H₂O, ACN) to give I-31 (43.8 mg, 21.1 μmol, 20% yield) as a white solid.

### Example 6. Synthesis of I-32

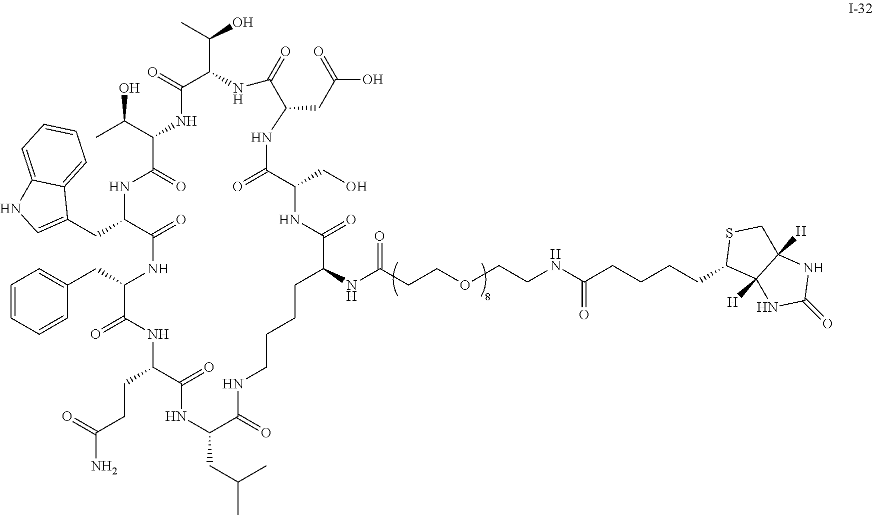

### General Procedure for Preparation of Compound 6.1

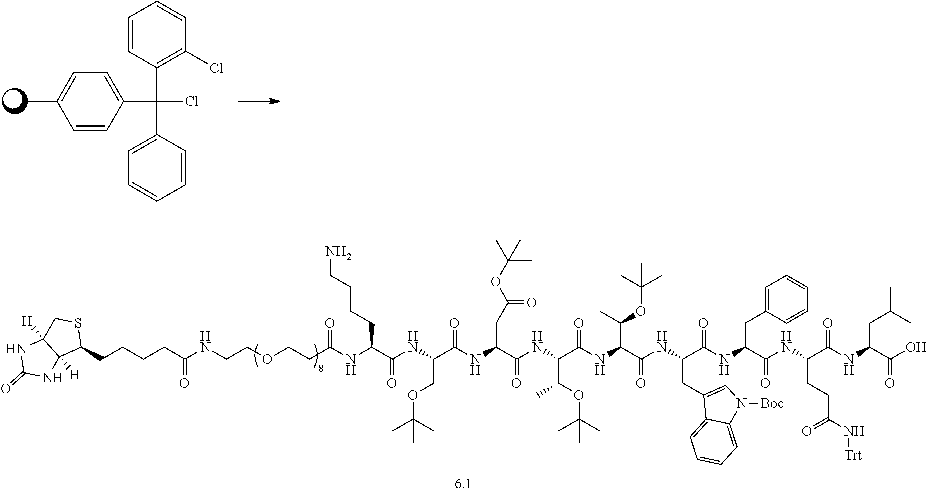

### Peptide Synthesis:

The peptide was synthesized using standard Fmoc chemistry.

Synthesis scale: 0.2 mmol.
1) Add DCM to the vessel containing CTC Resin and Fmoc-Leu-OH (70.68 mg, 200.00 μmol, 1 eq) with N₂ bubbling.
2) Drain and then DMF wash 30 sec with 3 times.
3) Add 20% piperidine/DMF and mix for 30 min.
4) Drain and then DMF wash 30 sec with 5 times.
5) Add Fmoc-amino acid solution and mix 30 seconds, then add coupling reagents with N₂ bubbling for about 1 hour.
6) Repeat step 2 to 5 for next amino acid coupling.

TABLE 6
Fmoc Amino Acids used in the Synthesis of I-32

| # | Materials | Coupling reagents |
|---|---|---|
| 1 | Fmoc-Leu-OH(1 eq) | DIEA (4 eq) |
| 2 | Fmoc-Gln(Trt)-OH (3 eq) | HBTU (2.9 eq) and DIEA (6 eq) |
| 3 | Fmoc-Phe-OH (3 eq) | HBTU (2.9 eq) and DIEA (6 eq) |
| 4 | Fmoc-Tyr(Boc)-OH (3 eq) | HBTU (2.9 eq) and DIEA (6 eq) |
| 5 | Fmoc-Thr(tBu)-OH (3 eq) | HBTU (2.9 eq) and DIEA (6 eq) |
| 6 | Fmoc-Thr(tBu)-OH (3 eq) | HBTU (2.9 eq) and DIEA (6 eq) |
| 7 | Fmoc-Asp(OtBu)-OH (3 eq) | HBTU (2.9 eq) and DIEA (6 eq) |
| 8 | Fmoc-Ser (tBu)-OH (3 eq) | HBTU (2.9 eq) and DIEA (6 eq) |
| 9 | Fmoc-Lys (Dde)-OH (1.5 eq) | HATU (1.5 eq) and DIEA (3 eq) |
| 10 | Peg₈ (1.5 eq) | HATU (1.5 eq) and DIEA (3 eq) |
| 11 | Biotin(1.5 eq) | HATU (1.5 eq) and DIEA (3 eq) |

20% piperidine in DMF was used for Fmoc deprotection for 30 min. The coupling reaction was monitored by ninhydrin test, and the resin was washed with DMF for 5 times.

### Peptide Cleavage and Work Up:

1) Add cleavage buffer (1% TFA/99% DCM) to the flask containing the side chain protected peptide at room temperature and stir for 0.5 hour and filtrated.
2) The filtration is added DIEA to neutralized and extracted with water twice.
3) Dry the crude peptide under vacuum 16 hours to give crude compound as white solid (300 mg, 64% yield).

### General Procedure for Preparation of Compound 6.2

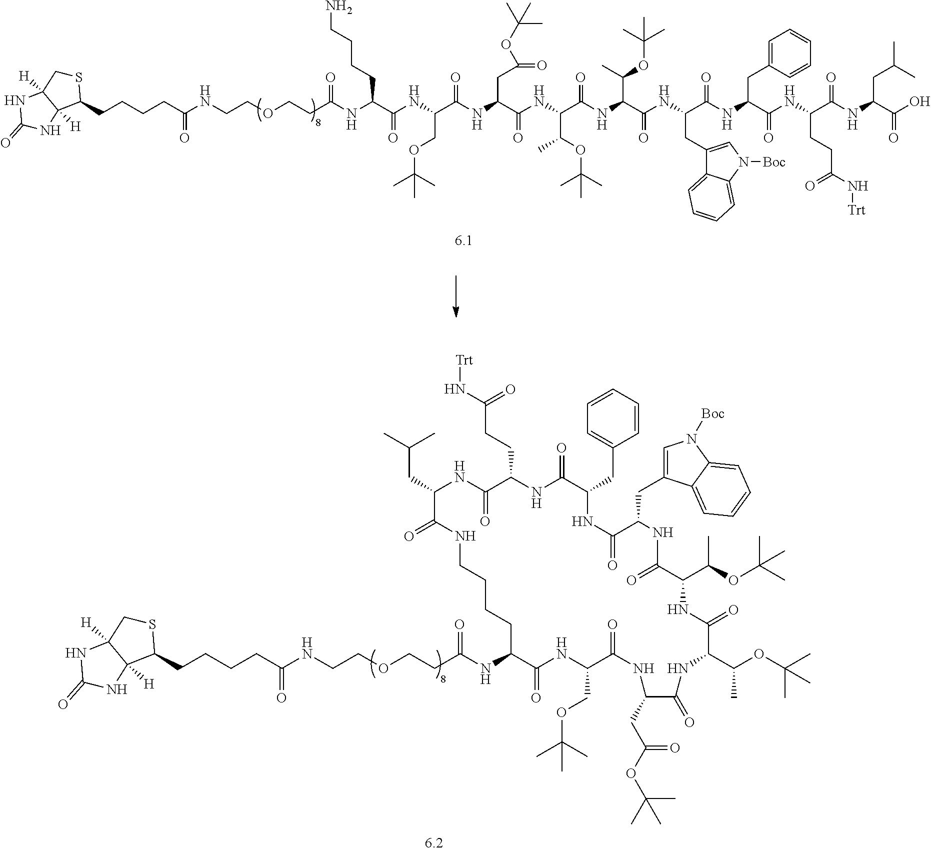

To a mixture of Compound 6.1 (300 mg, 128.10 μmol, 1 eq), HOBt (34.62 mg, 256.21 μmol, 2 eq) and TBTU (82.26 mg, 256.21 μmol, 2 eq) in DCM (200 mL) was added DIEA (66.22 mg, 512.41 μmol, 89.25 μL, 4 eq) dropwise at 25° C. under N₂. The mixture was stirred at 25° C. for 30 min. LC-MS showed compound 6.1 was consumed completely. The reaction mixture was was diluted with EtOAc 200 mL and extracted with water 200 mL (100 mL*2). The combined organic layers were washed with brine 200 mL (100 mL*2), dried over Na₂SO₄, filtered and concentrated under reduced pressure to give crude compound 6.2 (180 mg, 77.46 μmol, 60.47% yield).

### General Procedure for Preparation of I-32

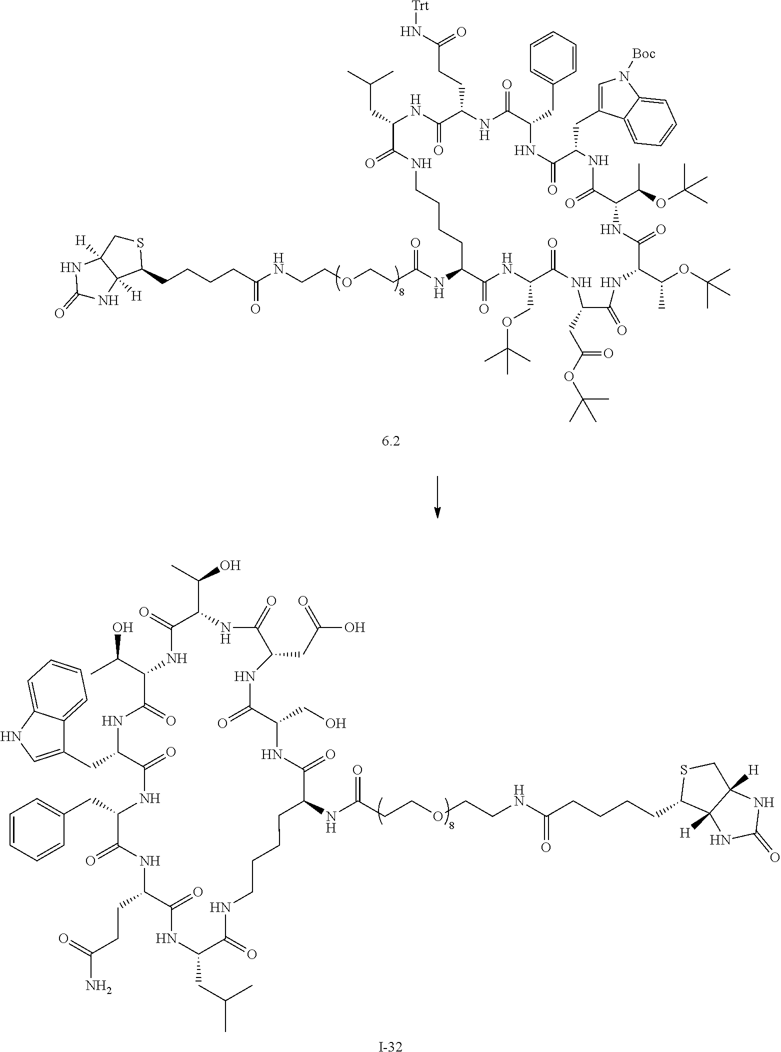

To a mixture of Compound 6.2 (180 mg, 77.46 μmol, 1 eq) was added 95% TFA/TIS/EDT/H2O in one portion at 25° C. The mixture was stirred at 25° C. for 2 hours. LC-MS showed Compound 6.2 was consumed completely. The peptide is precipitated with cold tert-butyl methyl ether and centrifuged (2 min at 5000 rpm). The residue was purified by prep-HPLC (0.075% TFA/H2O, ACN) to give I-32 (2 mg, 1.5% yield) as a white solid.

Other compounds in Table 1 were prepared and characterized similarly using technologies illustrated in the Examples in accordance with the present disclosure.

### Example 7. IgG FC Binding ELISA

Biotinylated test compounds or protein A control (Pierce: 29989) were added to streptavidin coated ELISA plates (Thermo Fisher: 15502) at 100 μL/well (PBS 0.05% tween-20-PBST, with 0.2% BSA). The plate was incubated for 2 h at 25° C. with rotation before the solutions were removed and the plate was washed twice with equal volumes of PBST. Fluorescein conjugated human IgG FC (50 nM in PBST with 0.2% BSA, Rockland: 009-0203) added to the plate at (100 μL/well) and incubated 25° C. for 45 min. The solutions were removed, the plate was washed twice with PBST, patted dry, and read with area scanning of each well (Biotek Synergy H1 microplate reader, 490/525 ex/em).

Table 7 shows the activity of selected compounds of this invention in the IgG FC binsing ELISA assay assay. The compound numbers correspond to the compound numbers in Table 1. Compounds having an activity designated as “A” provided a % signal relative to the standard Protein A at 1,000 nM of 90-120%; compounds having an activity designated as “B” provided a % signal relative to the standard Protein A at 1,000 nM of >4 and <90%; compounds of having an activity designated as “C” provided a % signal relative to the standard Protein A at 1,000 nM of 2-4%; and compounds having an activity designated as “D” provided a % signal relative to the standard Protein A at 1,000 nM of <2%.

TABLE 7
IgG FC binding ELISA Data

| | % signal of Protein |
|---|---|
| Compound ID | A at 1,000 nM |
| I-1 | C |
| I-2 | B |
| I-3 | C |
| I-4 | C |
| I-5 | C |
| I-7 | C |
| I-8 | B |
| I-9 | C |
| I-10 | C |
| I-12 | C |
| I-13 | C |
| I-14 | C |
| I-17 | A |
| I-18 | A |
| I-19 | A |

### Example 8. CD16a Binding of Recruited Antibodies

CD16a fluorescent labeling—CD16a158V (Sino Biologicals: 10389-H27H1) at 200 nM (PBST) and combined at an equal volume with Monolith NT His-Tag labeling kit, RED-tris-NTA dye (100 nM). The solution was incubated in the dark at 25° C. with rotation for 45 min before being spun down at 12000 rpm for 15 min to remove any uncomplexed reagent.

Biotinylated molecule immobilization—Biotinylated test compounds or control antibodies (IgG: Rockland 009-0602, IgM: Rockland, IgA: Rockland) were added to streptavidin coated ELISA plates (Thermo Fisher: 15502) at 50 μL/well (PBST with 0.2% BSA). The plate was incubated for 1 h at 25° C. with rotation before the solutions were removed and the plate was washed three times with PBST.

Serum antibody recruitment—Pooled normal human serum (Innovative Research, lot: 20966) was thawed on ice and spun down at 12000 rpm at 4° C. for 10 min prior to use to removed debris and precipitated protein. The supernatant was used to make a 2.5% solution or prepared as a dilution series (PBST with 0.2% BSA). The serum solution was added to the plate (50 μL/well) and incubated for 1 h with rotation. The solutions were removed and the plate was washed three times with PBST.

CD16a binding of recruited antibodies—The labeled CD16a was then added to the wells at 25 nM and incubated for 45 min. The solutions were removed and the plate was washed twice with PBST. The plates were then read with area scanning of each well (Biotek Synergy H1 microplate reader, 650/665 ex/em).

Table 8 shows the activity of selected compounds of this invention in the CD16a Binding of recruited Antibodies assay. The fluorescence readout is normalized to biotinylated IgG and the corresponding biotinylated molecule immobilized at 250 nM. The compound numbers correspond to the compound numbers in Table 1. Compounds having an activity designated as “A” provided a fluorescence readout of >2; compounds having an activity designated as “B” provided a fluorescence readout fluorescence readout of 1.5-2; compounds of having an activity designated as “C” provided a fluorescence readout of 1.0-1.5; and compounds having an activity designated as “D” provided a fluorescence readout of <1.

TABLE 8
CD16a Binding of recruited Antibodies Assay Data

| Compound ID | Fluoresence readout at 250 nM |
|---|---|
| I-17 | A |
| I-18 | C |
| I-19 | B |

### Example 9. ADCC Reporter Assay

LNCaP cells (human prostate cancer derived, ATCC: CRL-1740) were detached and re-suspended in 1% BSA in RPMI. Cell solution was filtered to remove cell aggregate (polystyrene tube with cell strainer, Corning: 352235) before being counted (Life Technologies Countless II cell counter) and plated (10,000 cells in 25 μL/well; Corning plates: 3917). The plated was centrifuged (5 min, 300×g) and the compound solutions (25 μL/well) were added immediately after. The antibody solutions (25 μL/well, IgG1 FC-Thermo Fisher: 10702HNAH5; Dintrophenyl-KLH polyclonal antibody-Thermo Fisher: A-6430) were added following a 45 min incubation at 37° C. The plate was then incubated with the antibody solution for 45 min to allow for cell-ARM-antibody ternary complex formation before the addition of the effector cells (60 K in 25 μL/well, ADCC reporter cells-Promega kit: G7018). The plate was centrifuged (5 min, 300×g) and effector cells were incubated for an 5 h at 37° C. Following the induction period the plate was equilibrated to 25° C. followed the addition of the luciferase substrate (75 uL/well, 1 vial in 10 mL of Bio-Glo assay buffer, Promega kit: G7018). The plate was then centrifuged (5 min, 300×g) and the luminescence was measured (Biotek Synergy H1 microplate reader).

Results from assays using various cells (LNCap, 22Rv1) demonstrated that provided compounds can effectively induce ADCC. An example set of data are present below:

LNCaP target cells and reporter cells with CD16a-158V/V variant

| I-11 | Luminescence (background substracted) | | | | | |
|---|---|---|---|---|---|---|
| [nM] | anti-DNP (20 ug/mL) | | IgG1 FC (200 nM) | | IgG1 FC (20 nM) | |
| 2000 | 1374.5 | 1253.5 | 1372 | 1118 | 151.5 | 163.5 |
| 400 | 1842.5 | 1696.5 | 1827 | 1494 | 797.5 | 679.5 |
| 80 | 1635.5 | 1605.5 | 1708 | 1823 | 1251.5 | 1037.5 |
| 16 | 934.5 | 855.5 | 1216 | 1040 | 667.5 | 621.5 |
| 3.2 | 128.5 | 86.5 | 172 | 189 | 165.5 | 123.5 |
| 0 | 15.5 | −15.5 | 8 | −8 | 46.5 | −46.5 |

*The average of antibody/target cells/reporter cell wells without compound were subtracted as background for each of the indicated antibody treatment groups.

### Example 10. Natural Killer (NK) Cell Activation Assay

Biotinylated test compounds or IgG (Rockland: 009-0602) were added to streptavidin coated ELISA plates (Thermo Fisher: 15502) at 100 μL/well (PBS 0.05% tween-20-PBST, with 0.2% BSA). The plate was incubated for 2 h at 25° C. with rotation before the solutions were removed and the plate was washed twice with equal volumes of PBST. Solutions of IgG1 FC (Thermo Fisher: 10702HNAH5) were then added to the wells containing the universal ABT and the plate was incubated for an additional 1 h before being washed twice with equal volumes of PBST and patted dry. Human PBMCs (Astrate Biologics: 1001) were thawed and cultured in 4% low IgG FBS (Corning: 35-073-CV) in RPMI with IL-2 (100 U/mL, Prospec: cyt-209) for 18 h prior to use. Immediate before addition to the ELISA plate, the cells were centrifuged (5 min, 300×g) and re-suspended in fresh media without IL-2. The PBMCs were then added to the plate (200 K cells in 100 μL/well), centrifuged (5 min, 300×g), and incubated at 37° C. for 5 h. Following incubation, the solutions were removed, the wells were washed once (1% BSA in PBS with 2 mM EDTA) and the combined solutions were centrifuged (5 min, 300×g). The supernatant was removed and the cells were re-suspended in buffer (100 μL/sample, 1% BSA in PBS with 2 mM EDTA) containing cell marker antibodies (1:200; anti-CD107a—BioLegend: 328626; anti-CD56—BioLegend: 318347). The antibodies were incubated at 4° C. for 30 min, diluted to 500 μL, centrifuged (5 min, 300×g) and resuspended in buffer (200 μL/sample, 1% BSA in PBS with 2 mM EDTA) and analyzed by flow cytometry (BD FACSCelesta). Data was collected using BD DIVA software and analyzed with FloJo.

Compound I-17 was active in the NK cell activation assay, showing a relative population of CD107a+ NK cells of 93% versus the control utilizing IgG.

### Example 11. Provided Compounds Form Complexes with Antibodies and Target Cells

As demonstrated herein, in some embodiments, provided compounds can recruit antibodies to target cells to form ternary complexes and induce immune activities. Various technologies are suitable for assessing complex formation. One example such assay was described below. Those skilled in the art appreciate that one or more parameters and/or conditions may be adjusted and other assays/reagents/conditions, etc., can also be utilized.

Adherent cells (LNCap, CRL-1740, for example results below) were harvested using Accumax (Sigma-Aldrich A7089-100 ml). Compounds were diluted in DMSO (MP 191418) to 1000× the starting concentration used in the assay into a 96 well polypropylene plate (Corning 3357). They were then serially diluted in 1/2 log increments to generate 8-12 concentrations in DMSO (assay dependent). These DMSO stocks were then step diluted 1/10 into PBS (VWR Cat. #20012043). The step diluted compound range was then added into the polypropylene assay plate 1/100 the volume of the assay volume. The cells used for the assay were counted and centrifuged and resuspended at a concentration of 100,000 cells per 200 ul in Flow buffer: 1% BSA (American Bio AB01088-00100); 0.5 mM EDTA (VWR 45001-122); PBS (VWR Cat. #20012043) with 20 ug/ml rabbit anti-DNP Alexa 488 (Thermo Fisher Scientific A11097). The cells were then added into the polypropylene plate with the step diluted compounds and incubated at 4 C 30 min. At the end of the incubation, the cells were centrifuged and washed 2× with Flow buffer containing 0.5% Tween 20 (BP337-500). Samples were analyzed on a BD FacsCelesta. Mean fluorescence was analyzed using Graphpad Prism and curves were fit using log (inhibitor) vs. response-Variable slope (four parameters).

Example results were presented below:

| Compound ID | EC50 (nM) |
|---|---|
| I-11 | 6.6 |
| I-33 | 3 |
| I-34 | 8.7 |
| I-16 | 7.4 |

### Example 12. Provided Compounds Can Induce ADCP

As demonstrated herein, in some embodiments, provided compounds can recruit antibodies to target cells to form ternary complexes and induce, generate, encourage and/or promote ADCP. Various technologies are suitable for assessing ADCP. One example such assay was described below. Those skilled in the art appreciate that one or more parameters and/or conditions may be adjusted and other assays/reagents/conditions, etc., can also be utilized.

### Primary Monocyte or THP Cell ADCP Assay

### Bead Preparation

Fluorescent beads with attached streptavidin (Invitrogen, cat #P35372) were thoroughly mixed by vortexing for 1 min. Ten microliters of the bead suspension per treatment type were dispensed in Eppendorf tubes, and washed with 1 ml PBS 1% BSA, and then re-suspended in 500 μL of 1% BSA in PBS. Test compounds were added to bead suspensions to a final concentration of 50 μM, and biotinylated human IgG whole molecule (Rockland 009-0602) final concentration of 20 ug/mL. Tubes were incubated at room temperature for 1 hr on a rocking platform. Following incubation, beads were pelleted in a microcentrifuge at 12,000×g for 5 minutes at room temperature. Supernatants were carefully aspirated and beads were washed 5 times by re-supending in 1 ml of PBS 1% BSA, and centrifuging as described above. Finally, beads were re-suspended in 500 ul of PBS 1% BSA and used in ADCP assay.

### Primary Monocyte Enrichment by Adhesion

PBMC, freshly isolated from whole blood (Bioreclamation IVTO) using Ficoll-Paque density centrifugation were re-suspended in serum-free, supplemented RPMI at 2× 10⁶ cells/mL. Ten milliliters of the resulting cell suspension was seeded into 75-cm² tissue culture flask (Denville Scientific TC9341) and incubated 2-3 hr at 37° C. 5% CO₂ to allow cell adhesion. Following incubation, media was decanted and surface of the flask was carefully washed twice, each time with 10 ml serum-free, supplemented RPMI to remove any residual non-adherent cells. To remove adherent cells, 10 mL of ice-cold 2.5 mM EDTA/PBS solution was added for 10 min on ice. Cells were transferred to a 15-ml conical tube and centrifuge 10 min at 300×g, room temperature, to remove the EDTA/PBS solution. Monocytes were then re-suspended in RPMI 10% FCS at 5×10⁵ cells/mL to be used in ADCP assay. Monocyte purity was assessed by staining cells with antibodies to human CD3 FITC (Biolegend 300306), CD19 PeCy7 (Biolegend 302216), CD14 BV421 (BD Biosciences, 565283), and CD16 (Biolegend 302046).

### ADCP Assay Using Fresh Primary Monocytes or THP-1 Cells

Monocytes or THP-1 cells (TIB-202 ATCC) were seeded into 96 well round bottom 96 well plates at the density of 10\hat{ }5 cells per well in 200 μl, and 10 μL of fluorescent bead suspension prepared as described above was added to each well. Well contents were mixed by pipetting up and down using a multichannel pipet, and plates were incubated at 37° C. for 18 hrs. After incubation, well contents were transferred into flat bottom black 96 well plates (Costar #3358) and centrifuged for 2 minutes at 300×g to allow for better visualization of the cells. Images were taken using Zoe fluorescent cell imager (Biorad).

Example results were presented below:

Normalized Phagocytosis score in enriched
monocytes for the biotinylated uABT

| Beads alone | I-17 | I-17 + IgG | Biotinylated IgG |
|---|---|---|---|
| 0 | 2.5 | 4.1 | 12.4 |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1-10. (canceled)

11. A compound of one of formulae II-a, II-b, II-c, II-d, II-e or II-f: 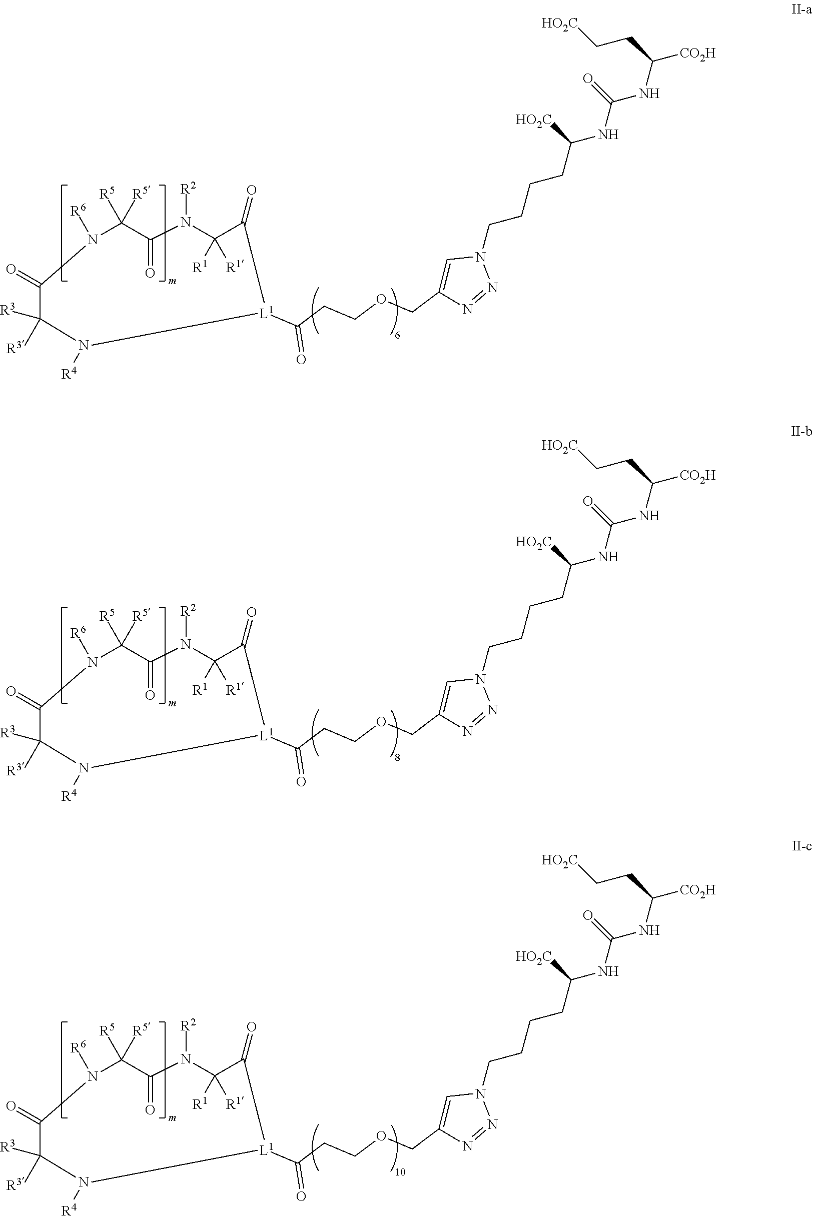 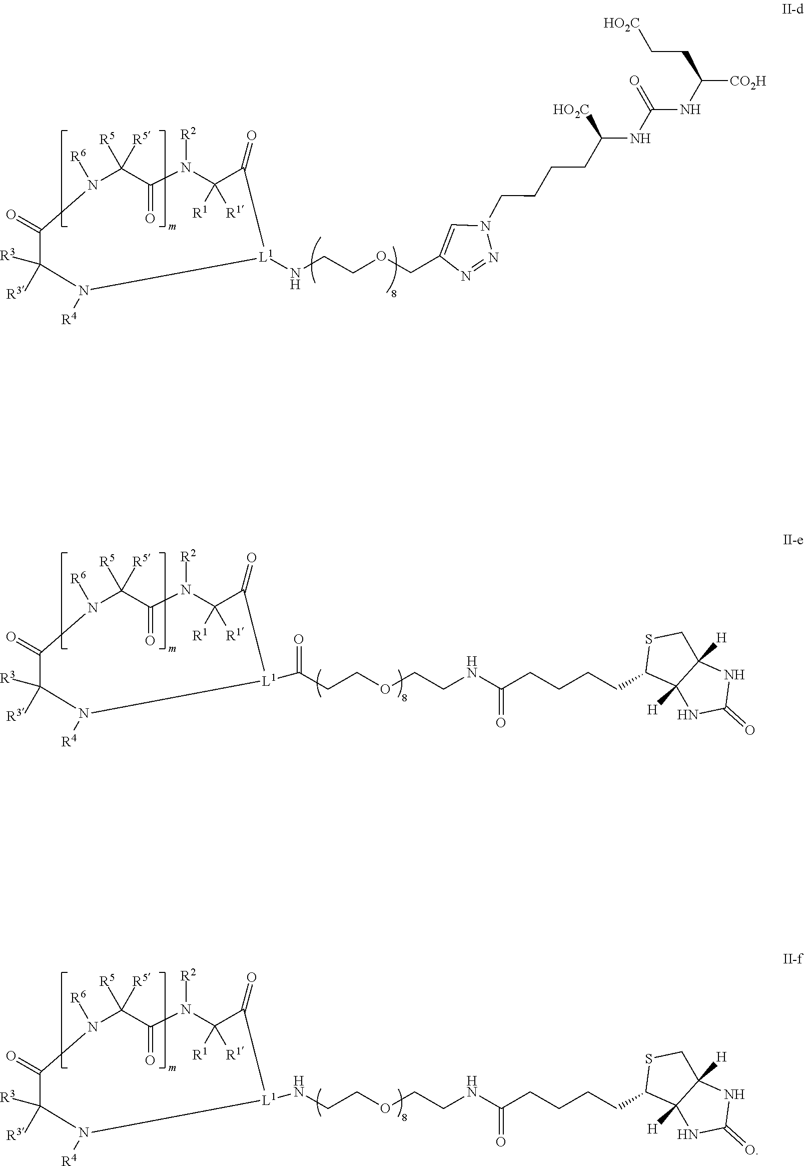
of a pharmaceutically acceptable salt thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen oxygen, of sulfur, or an 8-10 membered bicyclic heteroaromatic ring having 1-5 heteroatoms independently selected from nitrogen, oxygen, or sulfur; or:
R¹ and R^{1′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur;
R³ and R^{3′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur;
an R⁵ group and the R^{5′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen oxygen, or sulfur; or
two R⁵ groups are optionally taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen oxygen or sulfur;
each of R^{1′}, R^{3′} and R^{5′} is independently hydrogen or C₁₋₃ aliphatic;
each of R², R⁴ and R⁶ is independently hydrogen, or C₁₋₄ aliphatic, or:
R² and R¹ are optionally taken together with their intervening atoms to form a 4-8 membered saturated or partially unsaturated monocyclic heterocyclic ring having 1-2 heteroatoms independently selected from nitrogen, oxygen or sulfur,
R⁴ and R³ are optionally taken together with their intervening atoms to form a 4-8 membered saturated or partially unsaturated monocyclic heterocyclic ring having 1-2 heteroatoms independently selected from nitrogen, oxygen, or sulfur; or
an R⁶ group and its adjacent R⁵ group are optionally taken together with their intervening atoms to form a 4-8 membered saturated or partially unsaturated monocyclic heterocyclic ring having 1-2 heteroatoms independently selected from nitrogen, oxygen, or sulfur; and
L¹ is a trivalent linker moiety.

12-13. (canceled)

14. The compound of claim 1, or pharmaceutically acceptable salt thereof, wherein the compound is selected from those depicted in Table 1.

15. (canceled)

16. A compound having the structure of formula I-b: 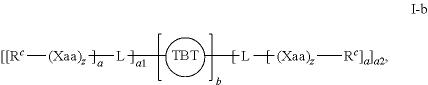
or a pharmaceutically acceptable salt thereof, wherein:
each Xaa is independently an amino acid residue;
each z is independently 1-50;
each L is independently a linker moiety;
TBT is a target binding moiety,
each R^{c} is independently -L^{a}-R′;
each of a1 and a2 is independently 0-200, wherein at least one of a1 and a2 is not 0;
b is 1-200;
each L^{a} is independently a covalent bond, or an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S— or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

17-19. (canceled)

20. The compound or pharmaceutically acceptable salt thereof of claim 16, wherein a is 1 and b is 1.

21. The compound of claim 20 or pharmaceutically acceptable salt thereof, wherein -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and
X¹² is Xaa^{A} or Xaa^{P},
wherein each Xaa^{A} is independently an amino acid residue whose side chain comprises an aromatic group, each Xaa^{P} is independently an amino acid residue whose side chain comprises a positively charged side chain, and each Xaa^{N} is independently an amino acid residue whose side chain comprises a negatively charged side chain.

22-26. (canceled)

27. The compound of claim 20 or pharmaceutically acceptable salt thereof, wherein -(Xaa)z- is or comprises —X³X⁴X⁵X⁶X⁷X⁸X⁹X¹⁰X¹¹X¹²— wherein:
at least two amino acid residues are connected through one or more linkages L^{b};
L is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N};
X¹² is Xaa^{A} or Xaa^{P}; and
wherein each Xaa^{A} is independently an amino acid residue whose side chain comprises an aromatic group, each Xaa^{P} is independently an amino acid residue whose side chain comprises a positively charged side chain, and each Xaa^{N} is independently an amino acid residue whose side chain comprises a negatively charged side chain.

28-33. (canceled)

34. The compound of claim 20 or pharmaceutically acceptable salt thereof, wherein -(Xaa)z- is or comprises —X³X⁴X⁵X⁶X⁷X⁸X⁹X¹⁰X¹¹X¹²—, wherein:
X² and X¹² are connected through one or more linkages L^{b};
L^{b} is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁴ is Xaa⁴;
X⁵ is Xaa^{A} or Xaa^{P};
X⁸ is Xaa^{N};
X¹¹ is Xaa^{A}; and
wherein each Xaa^{A} is independently an amino acid residue whose side chain comprises an aromatic group, each Xaa^{P} is independently an amino acid residue whose side chain comprises a positively charged side chain, and each Xaa^{N} is independently an amino acid residue whose side chain comprises a negatively charged side chain.

35-37. (canceled)

38. The compound of claim 20 or pharmaceutically acceptable salt thereof—claim 20 or pharmaceutically acceptable salt thereof—claim 20 or pharmaceutically acceptable salt thereof, wherein -(Xaa)z- is or comprises
—X²X³X⁴X⁵X⁶X⁷X⁸X⁹X¹⁰X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁵ is Xaa^{A} or Xaa^{P};
X⁸ is Xaa^{N};
X¹¹ is Xaa⁴; and
wherein each Xaa^{A} is independently an amino acid residue whose side chain comprises an aromatic group, each Xaa^{P} is independently an amino acid residue whose side chain comprises a positively charged side chain, and each Xaa^{N} is independently an amino acid residue whose side chain comprises a negatively charged side chain.

39-43. (canceled)

44. The compound of claim 20 or pharmaceutically acceptable salt thereof claim 20 or pharmaceutically acceptable salt thereof, wherein -(Xaa)z- is or comprises
—X²X³X⁴X⁵X⁶X⁷X⁸X⁹X¹⁰X¹¹X¹²X¹³X¹⁴X¹⁵X¹⁶—, wherein:
each of X², X³, X⁴, X⁵, X⁶, X⁷, X⁸, X⁹, X¹⁰, X¹¹, X¹², X¹³, X¹⁴, X¹⁵, and X¹⁶ is independently an amino acid residue;
at least two amino acid residues are connected through a linkage L^{b};
L^{b} is an optionally substituted bivalent group selected C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X³ is Xaa^{N};
X⁶ is Xaa^{A};
X⁷ is Xaa^{A} or Xaa^{P};
X⁹ is Xaa^{N};
X¹³ is Xaa^{A}; and
wherein each Xaa^{A} is independently an amino acid residue whose side chain comprises an aromatic group, each Xaa^{P} is independently an amino acid residue whose side chain comprises a positively charged side chain, and each Xaa^{N} is independently an amino acid residue whose side chain comprises a negatively charged side chain.

45-65. (canceled)

66. A pharmaceutical composition comprising a compound according to claim 14 or a pharmaceutically acceptable salt thereof, and a pharmaceutically acceptable carrier, adjuvant, or vehicle.

67. A method for recruiting a plurality of antibodies to a target site, comprising contacting a target site with a compound of claim 14 or a pharmaceutically acceptable salt thereof, wherein the compound recruits a plurality of antibodies to the target site.

68-74. (canceled)

75. A method of redirecting endogenous antibodies selectively to a diseased cell, thereby inducing antibody-directed, cell-mediated cytotoxicity or ADCP in said diseased cell in a patient comprising administering to said patient a compound of claim 14, or a pharmaceutical composition thereof.

76. A method of redirecting endogenous antibodies selectively to a diseased cell, thereby inducing antibody-directed, cell-mediated cytotoxicity in said diseased cell in a biological sample comprising contacting said biological sample with a compound or pharmaceutically acceptable salt thereof of claim 14, or a pharmaceutical composition thereof.

77-78. (canceled)

79. A method of treating a disorder, disease, or condition in a subject comprising administering to said subject a compound or pharmaceutically acceptable salt thereof according to claim 14, or a pharmaceutical composition thereof.

80-82. (canceled)

83. The compound or pharmaceutically acceptable salt thereof of claim 14, wherein the compound has the chemical structure:

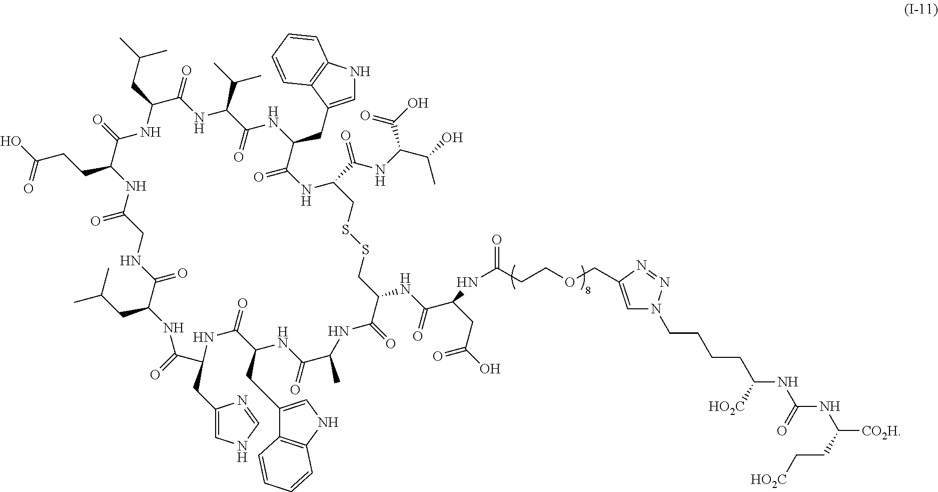

84. The compound or pharmaceutically acceptable salt thereof of claim 14, wherein the compound has the chemical structure:

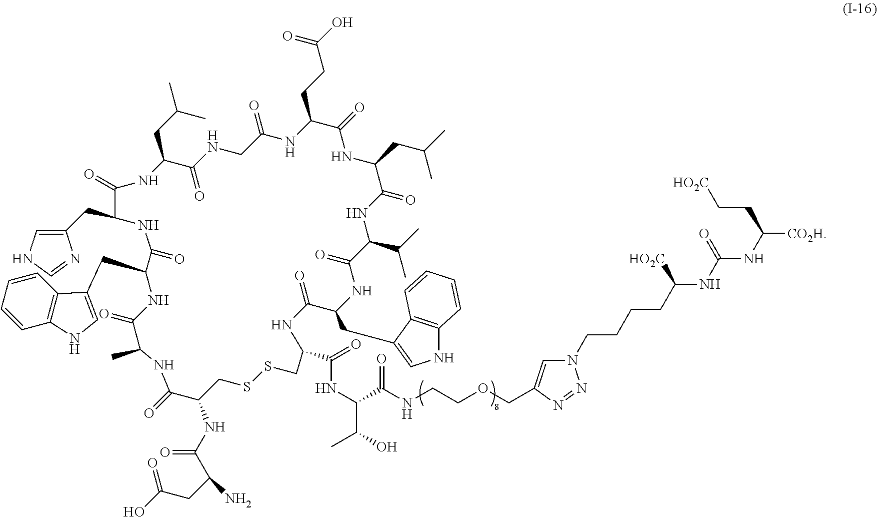

85. The compound or pharmaceutically acceptable salt thereof of claim 1, wherein the compound has the chemical structure:

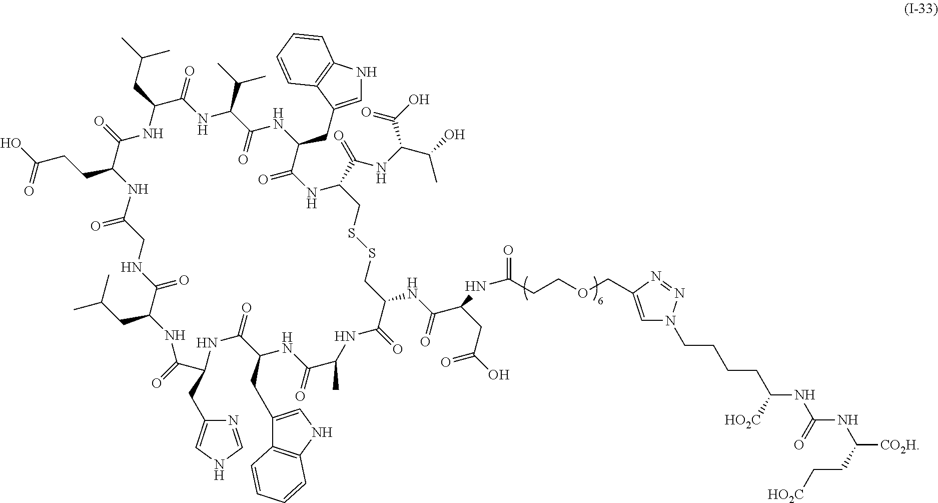

86. The compound or pharmaceutically acceptable salt thereof of claim 14, wherein the compound has the chemical structure: [chemical structure]
